(19) United States
(12) Patent Application Publication
(10) Pub. No.: US 2022/0098143 A1
(43) Pub. Date: Mar. 31, 2022

(54) CAPSAICIN AND TRPV1 MODULATOR COMBINATIONS AND METHODS OF USE THEREOF

(71) Applicant: Pano Therapeutics, Inc., San Francisco, CA (US)

(72) Inventor: Francesca Fieni, San Francisco, CA (US)

(21) Appl. No.: 17/548,281

(22) Filed: Dec. 10, 2021

Related U.S. Application Data
(63) Continuation of application No. PCT/US2020/037029, filed on Jun. 10, 2020.
(60) Provisional application No. 62/860,183, filed on Jun. 11, 2019.

(51) Int. Cl. C07C 233/20; C07C 69/734; C07C 69/708; C07D 295/033; C07D 401/12; C07D 401/04; C07D 211/08; C07D 311/68; C07D 217/02

(52) U.S. Cl. CPC C07C 233/20; C07C 69/734; C07C 69/708; C07D 295/033; C07D 217/02; C07D 401/04; C07D 211/08; C07D 311/68; C07D 401/12

(57) ABSTRACT

Provided herein are combinations of capsaicin and TrpV1 modulators that are useful for treating capsaicin-responsive diseases or disorders.

### CROSS-REFERENCE

This application is a continuation application which claims the benefit of International Application No. PCT/US2020/037029 filed Jun. 10, 2020 which claims the benefit of U.S. Provisional Application No. 62/860,183, filed Jun. 11, 2019, which application is incorporated herein by reference.

### BACKGROUND

A need exists in the medicinal arts for the effective treatment of capsaicin-responsive diseases and disorders. Such diseases and disorders include, but are not limited to, a cell proliferative disease (e.g., a cancer), obesity, diabetes, a bacterial infection, a cardiovascular disease, a neurodegenerative disease or disorder, an inflammatory disease or disorder, a comorbidity, autoimmune disease, hypercholesterolemia, and/or mood disorders.

### BRIEF SUMMARY

In an aspect, provided herein is a composition, comprising:
(i) a compound of structural Formula (I):

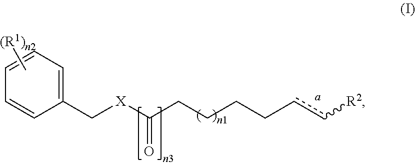

or an isotopic variant thereof; or a metabolite, pharmaceutically acceptable salt, solvate, or hydrate thereof,
   wherein:
[symbol] is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
(ii) at least one additional therapeutic agent selected from:

a fatty acid amide hydrolase (FAAH) receptor inhibitor, and

a specialized pro-resolving mediator (SPM), or a combination thereof; and
(iii) at least one excipient.

In an aspect, provided herein is a composition, consisting essentially of: (i) a compound of structural Formula (I):

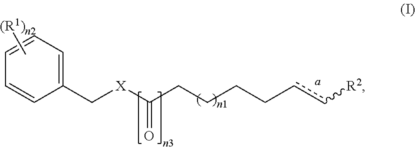

or an isotopic variant thereof; or a metabolite, pharmaceutically acceptable salt, solvate, or hydrate thereof,
   wherein:
[symbol] is a single bond or double bond;
n1 and n2 are independently an integer from 0 to 2;
n3 is 0 or 1;
X is O, NH, or S;
R² is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
(ii) a transient receptor potential cation channel subfamily V member 1 (TrpV1) modulator; and
(iii) at least one excipient.

In an aspect, provided herein is a composition, comprising:
(i) a compound of structural Formula (I):

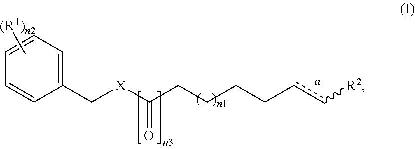

or an isotopic variant thereof; or a metabolite, pharmaceutically acceptable salt, solvate, or hydrate thereof,
   wherein:

a
[symbol] is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
(ii) a transient receptor potential cation channel subfamily V member 1 (TrpV1) modulator; and
(iii) at least one excipient,
   wherein when the compound of structural Formula (I), or an isotopic variant thereof; or a metabolite, pharmaceutically acceptable salt, solvate, or hydrate thereof, is capsaicin, then the capsaicin is present in the composition in an amount of greater than about 40% (w/v).

In some embodiments, when the compound of structural Formula (I), or an isotopic variant thereof; or a metabolite, pharmaceutically acceptable salt, solvate, or hydrate thereof, is capsaicin, guaiacol, eugenol, Zingerone, civamide, nonivamide, nuvanil, olvanil, NE-19550, NE-21610, or NE-28345, then the capsaicin, guaiacol, eugenol, Zingerone, civamide, nonivamide, nuvanil, olvanil, NE-19550, NE-21610, or NE-28345 are present in the composition in an amount of from about 40% (w/v) to about 50% (w/v).

In an aspect, provided herein is a composition, comprising:
(i) a compound of structural Formula (I):

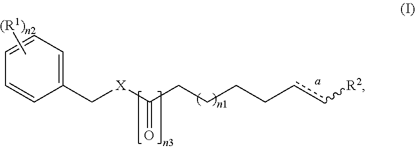

or an isotopic variant thereof; or a metabolite, pharmaceutically acceptable salt, solvate, or hydrate thereof,
   wherein:
[symbol] is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
(ii) a TrpV1 modulator; and
(iii) at least one excipient,
wherein the compound of Formula (I), isotopic variant thereof; or a metabolite, pharmaceutically acceptable salt, solvate, or hydrate thereof, is not capsaicin; or
wherein the TrpV1 modulator is not a fatty acid.

In some embodiments, the at least one TrpV1 modulator is a TrpV1 antagonist, a TrpV1 competitive agonist, or combination thereof

In some embodiments, the at least one TrpV1 modulator is a triglyceride or spilanthol.

In some embodiments, the at least one TrpV1 modulator is a fatty acid, triglyceride, or spilanthol.

In some embodiments, the fatty acid is a polyunsaturated fatty acid (PUFA).

In some embodiments, the PUFA is anandamide

In some embodiments, the fatty acid is an omega-9 fatty acid.

In some embodiments, the fatty acid is oleic acid or erucic acid.

In some embodiments, the at least one additional therapeutic agent is an FAAH receptor inhibitor.

In some embodiments, the FAAH receptor inhibitor is SSR411298.

In some embodiments, the at least one additional therapeutic agent is an SPM.

In some embodiments, the SPM is maresin.

In some embodiments, the pharmaceutical composition is in a form for oral dosing or administration.

In some embodiments, the form is a suspension or a solution.

In some embodiments, the form is a solid dosage form.

In some embodiments, the form is a tablet or a capsule.

In some embodiments, the pharmaceutical composition is in a form for inhalation, nebulization, or nasal delivery.

In some embodiments, the pharmaceutical composition is in a form for inhalation administration or dosing by vaporizer.

In some embodiments, the pharmaceutical composition is in a form for parenteral administration or dosing.

In some embodiments, the parenteral administration or dosing is injection, infusion, or implantation.

In some embodiments, the injection is intravenous, intramuscular, subcutaneous, or intradermal.

In some embodiments, the pharmaceutical composition is in a form for dosing or administration by suppository.

In some embodiments, the pharmaceutical composition is in a form for cutaneous or transdermal administration.

In some embodiments, the pharmaceutical composition is in a form for sublingual or buccal administration.

In some embodiments, the compound of Formula (I), or an isotopic variant thereof; or a metabolite, pharmaceutically acceptable salt, solvate, or hydrate thereof is present in the composition in an amount of greater than about 10% (w/v).

In some embodiments, the compound of Formula (I), or an isotopic variant thereof; or a metabolite, pharmaceutically acceptable salt, solvate, or hydrate thereof is present in the composition in an amount of greater than about 20% (w/v).

In some embodiments, the compound of Formula (I), or an isotopic variant thereof; or a metabolite, pharmaceutically acceptable salt, solvate, or hydrate thereof is present in the composition in an amount of greater than about 30% (w/v).

In some embodiments, the compound of Formula (I), or an isotopic variant thereof; or a metabolite, pharmaceutically acceptable salt, solvate, or hydrate thereof is present in the composition in an amount of greater than about 40% (w/v).

In some embodiments, the compound of Formula (I), or an isotopic variant thereof; or a metabolite, pharmaceutically acceptable salt, solvate, or hydrate thereof is present in the composition in an amount of from about 10% (w/v) to about 50% (w/v). In some embodiments, the compound of Formula (I), or an isotopic variant thereof; or a metabolite, pharmaceutically acceptable salt, solvate, or hydrate thereof is present in the composition in an amount of from about 20% (w/v) to about 50% (w/v). In some embodiments, the compound of Formula (I), or an isotopic variant thereof; or a metabolite, pharmaceutically acceptable salt, solvate, or hydrate thereof is present in the composition in an amount of from about 30% (w/v) to about 50% (w/v). In some embodiments, the compound of Formula (I), or an isotopic variant thereof; or a metabolite, pharmaceutically acceptable salt, solvate, or hydrate thereof is present in the composition in an amount of from about 40% (w/v) to about 50% (w/v).

In an aspect, provided herein is a composition, comprising:
(i) a compound of structural Formula (I):

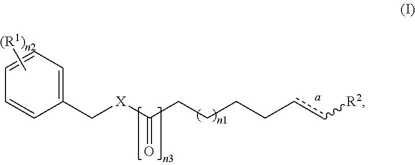

or an isotopic variant thereof; or a metabolite, pharmaceutically acceptable salt, solvate, or hydrate thereof,
   wherein:
[symbol] is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
(ii) a TrpV1 modulator; and
(iii) at least one excipient,
   wherein the composition is formulated for dosing or delivery by enteral or parenteral administration

In some embodiments, the composition is formulated as a liquid or a solid.

In some embodiments, the composition is formulated as a solid.

In some embodiments, the solid is a tablet, a capsule, a suppository, strip, or an implant.

In some embodiments, the tablet is a dissolvable tablet a bilayer tablet.

In some embodiments, the strip is a dissolvable strip.

In some embodiments, the composition is formulated as a liquid.

In some embodiments, the liquid is a solution, a suspension, an emulsion, a syrup, an elixir, a tincture, an ointment, a soft gel, an enema, foam, cream, lotion, or a gel.

In some embodiments, the composition is formulated for dosing or delivery by enteral administration.

In some embodiments, the enteral administration is oral or rectal administration.

In some embodiments, the composition is formulated for dosing or delivery by parenteral administration.

In some embodiments, the parenteral administration is sublingual administration, buccal administration, or administration by injection, infusion, implantation, or inhalation.

In an aspect, provided herein is a composition, comprising:
(i) a compound of structural Formula (I):

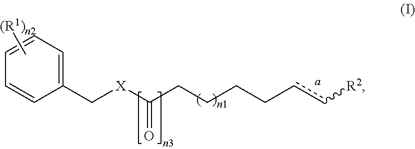

or an isotopic variant thereof; or a metabolite, pharmaceutically acceptable salt, solvate, or hydrate thereof,
   wherein: [symbol] is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1a} is hydrogen, —CF₃, —CCl₃, —CBr₃, —CI₃, —COOH, —CONH₂, substituted or unsubstituted alkyl, or substituted or unsubstituted heteroalkyl;
   (ii) a TrpV1 modulator; and
   (iii) at least one excipient.

In some embodiments, the solid is a tablet, a capsule, a suppository, strip, or an implant.

In some embodiments, the tablet is a dissolvable tablet or a bilayer tablet.

In some embodiments, the strip is a dissolvable strip.

In an aspect, provided herein is a composition, comprising:
(i) a compound of structural Formula (I):

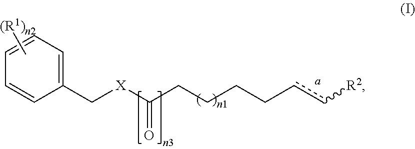

or an isotopic variant thereof; or a metabolite, pharmaceutically acceptable salt, solvate, or hydrate thereof,
   wherein:
[symbol] is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
(ii) a TrpV1 modulator; and
(iii) at least one excipient,
   wherein the compound of Formula (I), or an isotopic variant thereof; or a metabolite, pharmaceutically acceptable salt, solvate, or hydrate thereof, and the TrpV1 modulator are released sequentially in any order or have different drug release profiles.

In some embodiments, the compound of Formula (I), or an isotopic variant thereof; or a metabolite, pharmaceutically acceptable salt, solvate, or hydrate thereof, is released before the at least one additional therapeutic agent.

In some embodiments, the compound of Formula (I), or an isotopic variant thereof; or a metabolite, pharmaceutically acceptable salt, solvate, or hydrate thereof, is released after the at least one additional therapeutic agent.

In some embodiments, the solid is a tablet, a capsule, a suppository, strip, or an implant.

In some embodiments, the tablet is a dissolvable tablet or a bilayer tablet.

In some embodiments, the strip is a dissolvable strip.

In some embodiments, the compound of Formula (I), or an isotopic variant thereof; or a metabolite, pharmaceutically acceptable salt, solvate, or hydrate thereof is present in the composition in an amount of greater than about 10% (w/v).

In some embodiments, the compound of Formula (I), or an isotopic variant thereof; or a metabolite, pharmaceutically acceptable salt, solvate, or hydrate thereof is present in the composition in an amount of greater than about 20% (w/v).

In some embodiments, the compound of Formula (I), or an isotopic variant thereof; or a metabolite, pharmaceutically acceptable salt, solvate, or hydrate thereof is present in the composition in an amount of greater than about 30% (w/v).

In some embodiments, the compound of Formula (I), or an isotopic variant thereof; or a metabolite, pharmaceutically acceptable salt, solvate, or hydrate thereof is present in the composition in an amount of greater than about 40% (w/v).

In some embodiments, the compound of Formula (I), or an isotopic variant thereof; or a metabolite, pharmaceutically acceptable salt, solvate, or hydrate thereof is present in the composition in an amount of from about 10% (w/v) to about 50% (w/v). In some embodiments, the compound of Formula (I), or an isotopic variant thereof; or a metabolite, pharmaceutically acceptable salt, solvate, or hydrate thereof is present in the composition in an amount of from about 20% (w/v) to about 50% (w/v). In some embodiments, the compound of Formula (I), or an isotopic variant thereof; or a metabolite, pharmaceutically acceptable salt, solvate, or hydrate thereof is present in the composition in an amount of from about 30% (w/v) to about 50% (w/v). In some embodiments, the compound of Formula (I), or an isotopic variant thereof; or a metabolite, pharmaceutically acceptable salt, solvate, or hydrate thereof is present in the composition in an amount of from about 40% (w/v) to about 50% (w/v).

In some embodiments, the composition is a modified-release dosage form, a sustained-release dosage form, or a controlled-release dosage form.

In some embodiments, the composition is in modified-release dosage form.

In some embodiments, the modified-release dosage form is a delayed-release dosage form, an extended release dosage form, or a targeted release dosage form.

In some embodiments, the ratio of TrpV1 modulator to a compound of Formula (I), or an isotopic variant thereof; or a metabolite, pharmaceutically acceptable salt, solvate, or hydrate thereof, is about 1:1.

In some embodiments, the ratio of FAAH receptor inhibitor to a compound of Formula (I), or an isotopic variant thereof; or a metabolite, pharmaceutically acceptable salt, solvate, or hydrate thereof, is about 1:1.

In some embodiments, the ratio of SPM to a compound of Formula (I), or an isotopic variant thereof; or a metabolite, pharmaceutically acceptable salt, solvate, or hydrate thereof, is about 1:1.

In some embodiments, the compositions disclosed herein are formulated as a pharmaceutical composition.

In some embodiments, the at least one excipient is at least one pharmaceutically acceptable excipient.

In some embodiments, the compound of structural Formula (I) has structural Formula (I-A):

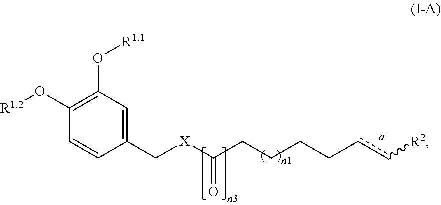

or an isotopic variant thereof; or a metabolite, pharmaceutically acceptable salt, solvate, or hydrate thereof,
   wherein:
R^{1.1} is hydrogen, halogen, —OR^{1.1A}, substituted or unsubstituted alkyl, or substituted or unsubstituted heteroalkyl;
R^{1.1} is hydrogen, halogen, —OR^{1.2A}, substituted or unsubstituted alkyl, or substituted or unsubstituted heteroalkyl;
R^{1.1A} and R^{1.2A} are independently hydrogen, —CF₃, —CCl₃, —CBr₃, —CI₃, —COOH, —CONH₂, substituted or unsubstituted alkyl, or substituted or unsubstituted heteroalkyl;

In some embodiments, R^{1.1} is CH₃; and R^{1.2} is OH.

a

In some embodiments, X is O. In some embodiments, X is NH. In some embodiments, [symbol] is a double bond. In some embodiments, [symbol] is a single bond. In some embodiments, n1 is 2. In some embodiments, n1 is 1. In some embodiments, n1 is 0. In some embodiments, R² is unsubstituted alkyl. In some embodiments, R² is —CH(CH₃)₂ or —CH₃.

In some embodiments, the compound is at least one of:

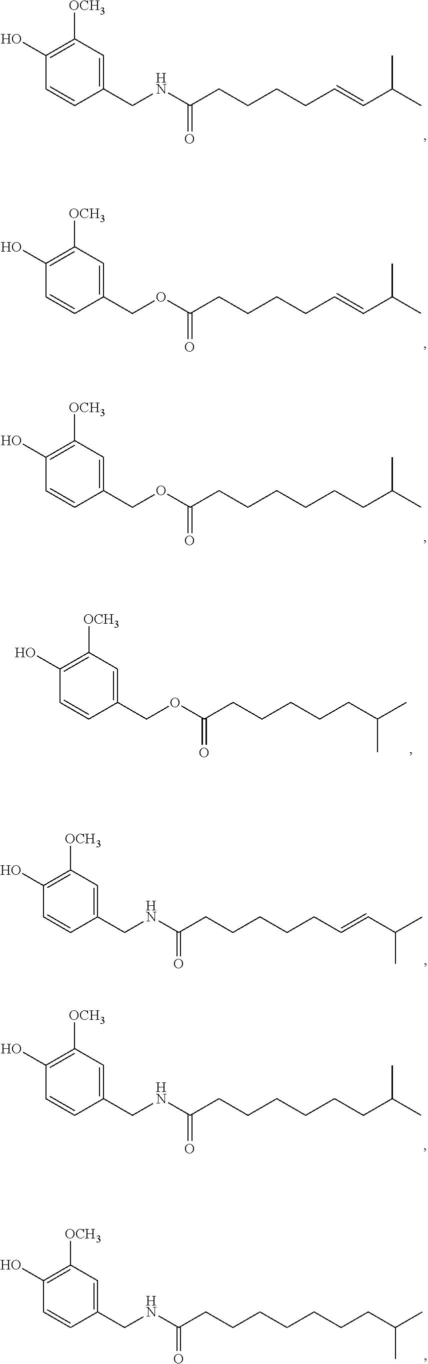

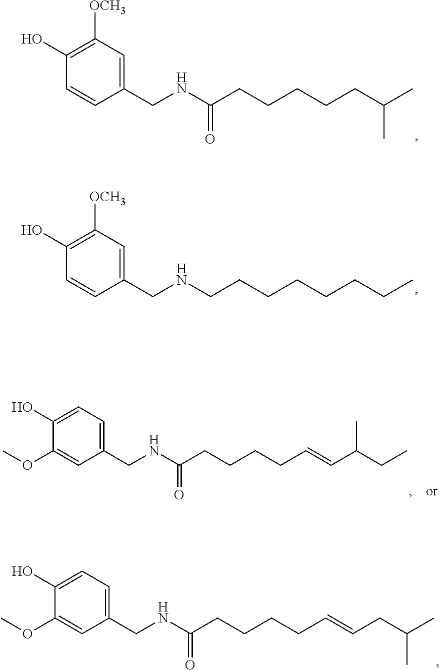

or an isotopic variant thereof; or a metabolite, pharmaceutically acceptable salt, solvate, or hydrate thereof.

In some embodiments, the compound is:

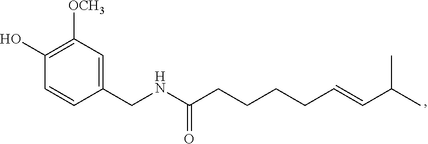 [SMILES: COc1cc(CNC(=O)CCCC/C=C/C(C)C)ccc1O]

or an isotopic variant thereof; or a metabolite, pharmaceutically acceptable salt, solvate, or hydrate thereof

In an aspect, provided herein is a method of treating and/or preventing a capsaicin-responsive disease or disorder, comprising administering to a subject in need thereof the pharmaceutical composition disclosed herein.

In some embodiments, the disease or disorder is a cell proliferative disease, obesity, diabetes, a bacterial infection, a cardiovascular disease, a neurodegenerative disease or disorder, an inflammatory disease or disorder, a comorbidity, an autoimmune disease, hypercholesterolemia or a mood disorder.

In some embodiments, the mood disorder is depression. In some embodiments, the inflammatory disease or disorder is rheumatoid arthritis. In some embodiments, the cell proliferative disease is a cancer. In some embodiments, the disorder is diabetes or obesity. In some embodiments, the comorbidity is chronic kidney disease, stroke, congestive heart failure, dementia, schizophrenia, hepatitis, autism spectrum disorder, HIV, or a combination thereof.

In another aspect, provided herein is a supplement, comprising:
(i) a compound of structural Formula (I):

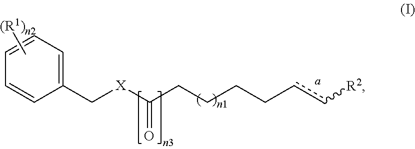

or an isotopic variant thereof; or a metabolite, pharmaceutically acceptable salt, solvate, or hydrate thereof,
   wherein:
[symbol] is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
(ii) at least one additional therapeutic agent selected from:
   a fatty acid amide hydrolase (FAAH) receptor inhibitor, and
   a specialized pro-resolving mediator (SPM), or a combination thereof; and
(iii) at least one excipient.

In some embodiments, the compound of structural Formula (I) has structural Formula (I-A):

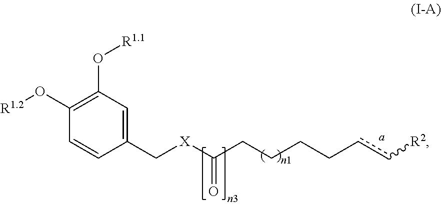

or an isotopic variant thereof; or a metabolite, pharmaceutically acceptable salt, solvate, or hydrate thereof,
   wherein:
R^{1.1} is hydrogen, halogen, —OR^{1.1A}, substituted or unsubstituted alkyl, or substituted or unsubstituted heteroalkyl;
R^{1.2} is hydrogen, halogen, —OR^{1.2A}, substituted or unsubstituted alkyl, or substituted or unsubstituted heteroalkyl; and
R^{1.1A} and R^{1.2A} are independently hydrogen, —CF₃, —CCl₃, —CBr₃, —CI₃, —COOH, —CONH₂, substituted or unsubstituted alkyl, or substituted or unsubstituted heteroalkyl.

In some embodiments, R^{1.1} is CH₃; and R^{1.2} is OH.

In some embodiments, X is O. In some embodiments, X is NH. In some embodiments, [symbol] is a a double bond. In some embodiments, [symbol] is a single bond. In some embodiments, n1 is 2. In some embodiments, n1 is 1. In some embodiments, n1 is 0. In some embodiments, R² is unsubstituted alkyl. In some embodiments, R² is —CH(CH₃)₂ or —CH₃.

In some embodiments, the compound is at least one of:

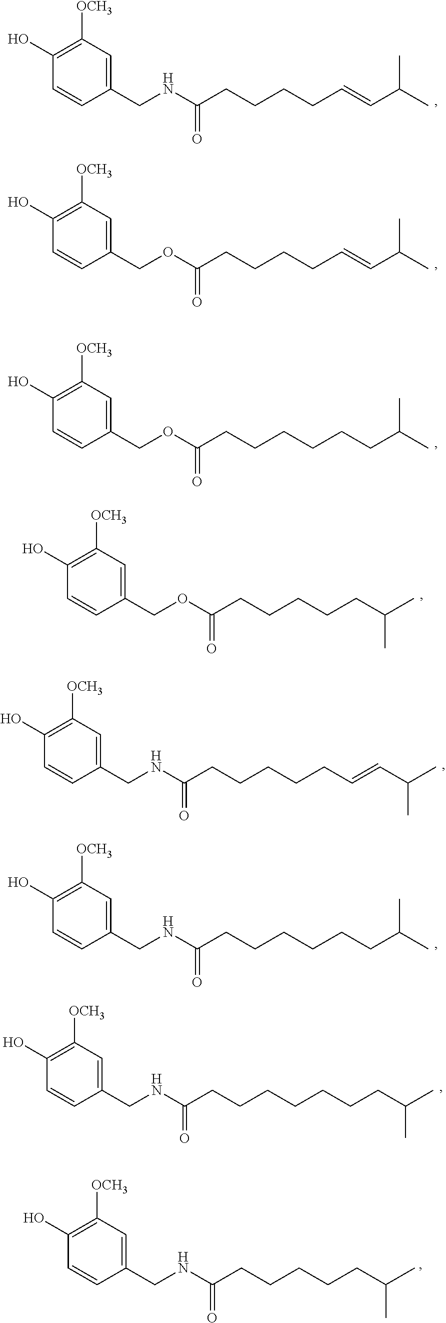

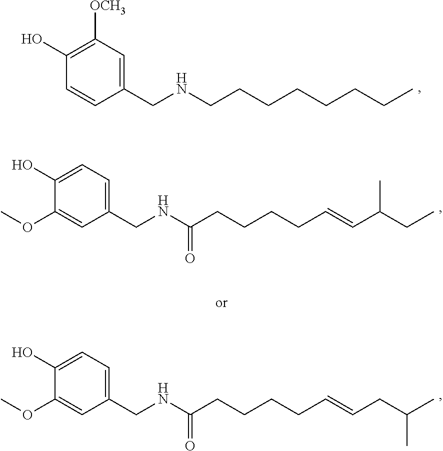

or an isotopic variant thereof; or a metabolite, pharmaceutically acceptable salt, solvate, or hydrate thereof.

In some embodiments, the compound is:

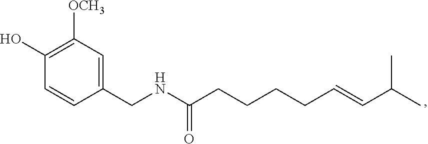 [SMILES: COc1cc(CNC(=O)CCCC/C=C/C(C)C)ccc1O]

or an isotopic variant thereof; or a metabolite, pharmaceutically acceptable salt, solvate, or hydrate thereof.

In some embodiments, the at least one additional therapeutic agent is a TrpV1 modulator.

In some embodiments, the at least one TrpV1 modulator is a TrpV1 antagonist, a TrpV1 competitive agonist, or combination thereof.

In some embodiments, the at least one TrpV1 modulator is a fatty acid, triglyceride, or spilanthol.

In some embodiments, the fatty acid is a polyunsaturated fatty acid (PUFA).

In some embodiments, the PUFA is anandamide

In some embodiments, the fatty acid is an omega-9 fatty acid.

In some embodiments, the fatty acid is oleic acid or erucic acid.

In some embodiments, the at least one additional therapeutic agent is a FAAH receptor inhibitor.

In some embodiments, the FAAH receptor inhibitor is SSR411298.

In some embodiments, the at least one additional therapeutic agent is a FAAH inhibitor.

In some embodiments, the FAAH inhibitor is a flavonoid. Exemplary flavonoids include, but are not limited to, myricetin, quercetin, kaempferol, luteolin, and apigenin. In some embodiments, the FAAH inhibitor is kaempferol.

In some embodiments, the at least one additional therapeutic agent is an SPM.

In some embodiments, the SPM is maresin.

In some embodiments, the supplement is in a form for oral dosing or administration. In some embodiments, the form is a suspension or a solution. In some embodiments, the form is a solid dosage form. In some embodiments, the solid dosage form is a tablet or a capsule.

In some embodiments, the supplement is in a form for inhalation, nebulization, or nasal delivery.

In some embodiments, the supplement is in a form for inhalation administration or dosing by vaporizer.

In some embodiments, the supplement is in a form for parenteral administration or dosing.

In some embodiments, the administration or dosing is parenteral injection.

In some embodiments, the injection is intravenous, intramuscular, subcutaneous, or intradermal.

In some embodiments, the supplement is in a form for dosing or administration by suppository.

In some embodiments, the supplement is in a form for cutaneous or transdermal administration.

In some embodiments, the supplement is in a form for sublingual or buccal administration.

In some embodiments, the compound of Formula (I), or an isotopic variant thereof; or a metabolite, pharmaceutically acceptable salt, solvate, or hydrate thereof is present in the composition in an amount of greater than about 10% (w/v).

In some embodiments, the compound of Formula (I), or an isotopic variant thereof; or a metabolite, pharmaceutically acceptable salt, solvate, or hydrate thereof is present in the composition in an amount of greater than about 20% (w/v).

In some embodiments, the compound of Formula (I), or an isotopic variant thereof; or a metabolite, pharmaceutically acceptable salt, solvate, or hydrate thereof is present in the composition in an amount of greater than about 30% (w/v).

In some embodiments, the compound of Formula (I), or an isotopic variant thereof; or a metabolite, pharmaceutically acceptable salt, solvate, or hydrate thereof is present in the composition in an amount of greater than about 40% (w/v).

In some embodiments, the compound of Formula (I), or an isotopic variant thereof; or a metabolite, pharmaceutically acceptable salt, solvate, or hydrate thereof is present in the composition in an amount of from about 10% (w/v) to about 50% (w/v). In some embodiments, the compound of Formula (I), or an isotopic variant thereof; or a metabolite, pharmaceutically acceptable salt, solvate, or hydrate thereof is present in the composition in an amount of from about 20% (w/v) to about 50% (w/v). In some embodiments, the compound of Formula (I), or an isotopic variant thereof; or a metabolite, pharmaceutically acceptable salt, solvate, or hydrate thereof is present in the composition in an amount of from about 30% (w/v) to about 50% (w/v). In some embodiments, the compound of Formula (I), or an isotopic variant thereof; or a metabolite, pharmaceutically acceptable salt, solvate, or hydrate thereof is present in the composition in an amount of from about 40% (w/v) to about 50% (w/v).

In some embodiments, the supplements disclosed herein are for use in the treatment of a capsaicin-responsive disease or disorder.

In some embodiments, the disease or disorder is a cell proliferative disease, obesity, diabetes, a bacterial infection, a cardiovascular disease, a neurodegenerative disease or disorder, an inflammatory disease or disorder, a comorbidity, an autoimmune disease, hypercholesterolemia or a mood disorder.

In some embodiments, the mood disorder is depression. In some embodiments, the inflammatory disease or disorder is rheumatoid arthritis. In some embodiments, the cell proliferative disease is a cancer. In some embodiments, the disorder is diabetes or obesity.

In an aspect, provided herein is a method of treating or preventing a capsaicin-responsive disease or disorder, comprising administering to a subject in need thereof:
(i) a compound of structural Formula (I):

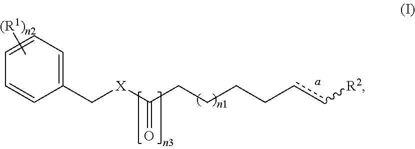

or an isotopic variant thereof; or a metabolite, pharmaceutically acceptable salt, solvate, or hydrate thereof,
   wherein:
[symbol] is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl; and
(ii) at least one additional therapeutic agent selected from:
   a transient receptor potential cation channel subfamily V member 1 (TrpV1) modulator,
   a fatty acid amide hydrolase (FAAH) receptor inhibitor; and
   a specialized pro-resolving mediator (SPM), or a combination thereof,
      wherein when the TrpV1 modulator is a fatty acid, then the compound of structural Formula (I) or an isotopic variant thereof; or a metabolite, pharmaceutically acceptable salt, solvate, or hydrate thereof, and the TrpV1 modulator are each individually formulated.

In some embodiments, R^{1.1 is CH}₃; and R^{1.2} is OH.

In some embodiments, X is O. In some embodiments, X is NH. In some embodiments, [symbol] is a double bond. In some embodiments, [symbol] is a single bond. In some embodiments, n1 is 2. In some embodiments, n1 is 1. In some embodiments, n1 is 0. In some embodiments, R² is unsubstituted alkyl. In some embodiments, R² is —CH(CH₃)₂ or —CH₃.

In some embodiments, the compound is at least one of:

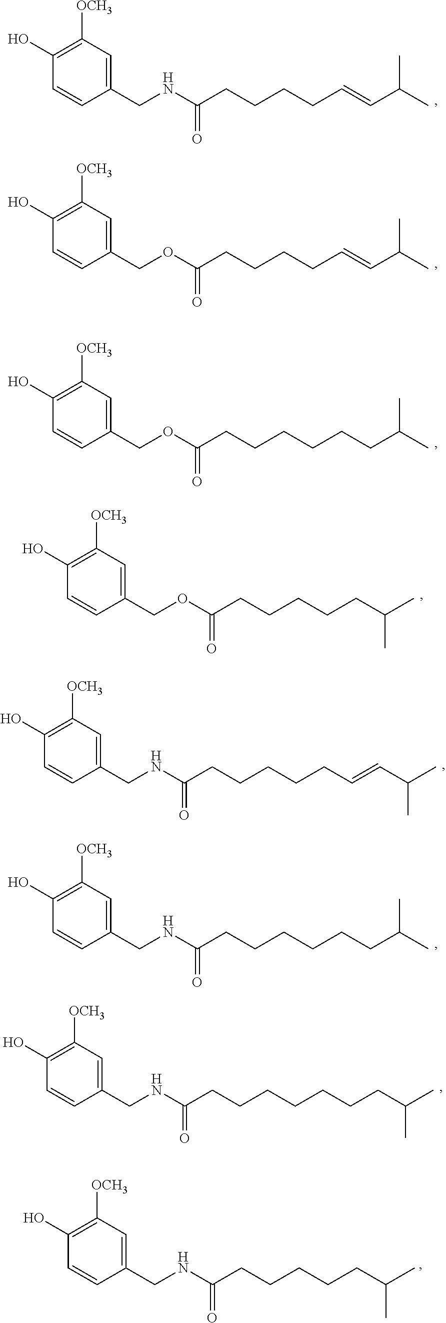

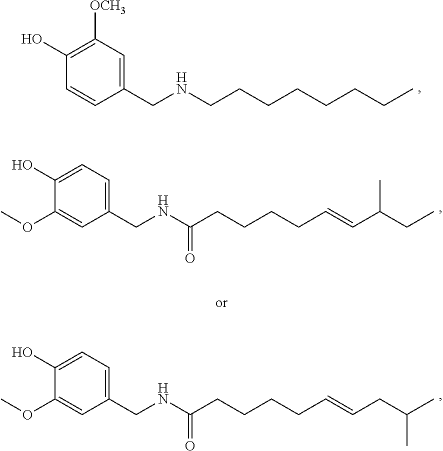

or an isotopic variant thereof; or a metabolite, pharmaceutically acceptable salt, solvate, or hydrate thereof.

In some embodiments, the compound is:

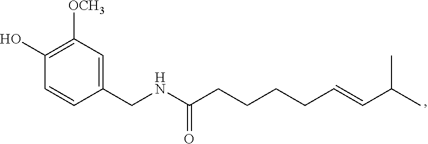 [SMILES: COc1cc(CNC(=O)CCCC/C=C/C(C)C)ccc1O]

or an isotopic variant thereof; or a metabolite, pharmaceutically acceptable salt, solvate, or hydrate thereof.

In some embodiments, the at least one additional therapeutic agent is a TrpV1 modulator.

In some embodiments, the TrpV1 modulator is a TrpV1 antagonist, a TrpV1 competitive agonist, or combination thereof.

In some embodiments, the TrpV1 modulator is a fatty acid triglyceride, or spilanthol.

In some embodiments, the fatty acid is a polyunsaturated fatty acid (PUFA).

In some embodiments, the PUFA is anandamide

In some embodiments, the fatty acid is an omega-9 fatty acid.

In some embodiments, the fatty acid is oleic acid or erucic acid.

In some embodiments, the at least one additional therapeutic agent is a FAAH receptor inhibitor.

In some embodiments, the FAAH receptor inhibitor is SSR411298.

In some embodiments, the at least one additional therapeutic agent is a FAAH inhibitor.

In some embodiments, the FAAH inhibitor is a flavonoid. Exemplary flavonoids include, but are not limited to, myricetin, quercetin, kaempferol, luteolin, and apigenin. In some embodiments, the FAAH inhibitor is kaempferol.

In some embodiments, the at least one additional therapeutic agent is an SPM.

In some embodiments, the SPM is maresin.

In some embodiments, the compound of structural Formula (I) or an isotopic variant thereof; or a metabolite, pharmaceutically acceptable salt, solvate, or hydrate thereof, is administered orally.

In some embodiments, the compound of structural Formula (I) or an isotopic variant thereof; or a metabolite, pharmaceutically acceptable salt, solvate, or hydrate thereof, is administered orally via a suspension or solution.

In some embodiments, the compound of structural Formula (I) or an isotopic variant thereof; or a metabolite, pharmaceutically acceptable salt, solvate, or hydrate thereof, is administered orally via a solid dosage form. In some embodiments, the solid dosage form is a tablet or capsule.

In some embodiments, the compound of structural Formula (I) or an isotopic variant thereof; or a metabolite, pharmaceutically acceptable salt, solvate, or hydrate thereof, is administered by inhalation, nebulization, or nasal delivery.

In some embodiments, the compound of structural Formula (I) or an isotopic variant thereof; or a metabolite, pharmaceutically acceptable salt, solvate, or hydrate thereof, is administered by inhalation via a vaporizer.

In some embodiments, the compound of structural Formula (I) or an isotopic variant thereof; or a metabolite, pharmaceutically acceptable salt, solvate, or hydrate thereof, is administered parenterally.

In some embodiments, the compound of structural Formula (I) or an isotopic variant thereof; or a metabolite, pharmaceutically acceptable salt, solvate, or hydrate thereof, is administered via parenteral injection, infusion, or implantation.

In some embodiments, the parenteral injection is intravenous, intramuscular, subcutaneous, or intradermal.

In some embodiments, the compound of structural Formula (I) or an isotopic variant thereof; or a metabolite, pharmaceutically acceptable salt, solvate, or hydrate thereof, is administered via a suppository.

In some embodiments, the compound of structural Formula (I) or an isotopic variant thereof; or a metabolite, pharmaceutically acceptable salt, solvate, or hydrate thereof, is administered cutaneous or transdermal delivery.

In some embodiments, the compound of structural Formula (I) or an isotopic variant thereof; or a metabolite, pharmaceutically acceptable salt, solvate, or hydrate thereof, is administered by sublingual or buccal delivery.

In some embodiments, the compound of Formula (I), or an isotopic variant thereof; or a metabolite, pharmaceutically acceptable salt, solvate, or hydrate thereof is present in the composition in an amount of greater than about 10% (w/v).

In some embodiments, the compound of Formula (I), or an isotopic variant thereof; or a metabolite, pharmaceutically acceptable salt, solvate, or hydrate thereof is present in the composition in an amount of greater than about 20% (w/v).

In some embodiments, the compound of Formula (I), or an isotopic variant thereof; or a metabolite, pharmaceutically acceptable salt, solvate, or hydrate thereof is present in the composition in an amount of greater than about 30% (w/v).

In some embodiments, the compound of Formula (I), or an isotopic variant thereof; or a metabolite, pharmaceutically acceptable salt, solvate, or hydrate thereof is present in the composition in an amount of greater than about 40% (w/v).

In some embodiments, the compound of Formula (I), or an isotopic variant thereof; or a metabolite, pharmaceutically acceptable salt, solvate, or hydrate thereof is present in the composition in an amount of from about 10% (w/v) to about 50% (w/v). In some embodiments, the compound of Formula (I), or an isotopic variant thereof; or a metabolite, pharmaceutically acceptable salt, solvate, or hydrate thereof is present in the composition in an amount of from about 20% (w/v) to about 50% (w/v). In some embodiments, the compound of Formula (I), or an isotopic variant thereof; or a metabolite, pharmaceutically acceptable salt, solvate, or hydrate thereof is present in the composition in an amount of from about 30% (w/v) to about 50% (w/v). In some embodiments, the compound of Formula (I), or an isotopic variant thereof; or a metabolite, pharmaceutically acceptable salt, solvate, or hydrate thereof is present in the composition in an amount of from about 40% (w/v) to about 50% (w/v).

In some embodiments, the ratio of the therapeutic agent to a compound of Formula (I), or an isotopic variant thereof; or a metabolite, pharmaceutically acceptable salt, solvate, or hydrate thereof, is about 1:1.

In some embodiments, the disease or disorder is a cell proliferative disease, obesity, diabetes, a bacterial infection, a cardiovascular disease, a neurodegenerative disease or disorder, an inflammatory disease or disorder, a comorbidity, an autoimmune disease, hypercholesterolemia or a mood disorder. In some embodiments, the mood disorder is depression. In some embodiments, the inflammatory disease or disorder is rheumatoid arthritis In some embodiments, the cell proliferative disease is a cancer. In some embodiments, the disorder is diabetes or obesity.

In some embodiments, the compound of Formula (I), or a pharmaceutically acceptable salt thereof, and the at least one additional therapeutic agent are administered simultaneously, approximately simultaneously, or sequentially, in any order.

In some embodiments, the compound of Formula (I), or a pharmaceutically acceptable salt thereof, and the at least one additional therapeutic agent are administered simultaneously or approximately simultaneously.

In some embodiments, the compound of Formula (I), or a pharmaceutically acceptable salt thereof, and the at least one additional therapeutic agent are administered sequentially.

In some embodiments, the compound of Formula (I), or a pharmaceutically acceptable salt thereof, is released before the at least one additional therapeutic agent.

In some embodiments, the compound of Formula (I), or a pharmaceutically acceptable salt thereof, is released after the at least one additional therapeutic agent.

An aspect described herein are compositions, comprising: (i) a compound of structural Formula (I):

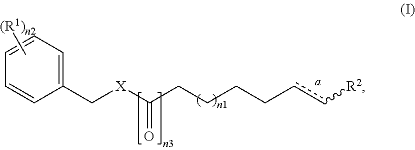

or an isotopic variant thereof; or a metabolite, pharmaceutically acceptable salt, solvate, or hydrate thereof,
   wherein:
[symbol] is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
(ii) a transient receptor potential cation channel subfamily V member 1 (TrpV1) partial agonist; and (iii) at least one excipient. In one feature, the compound of structural Formula (I), or an isotopic variant thereof; or a metabolite, pharmaceutically acceptable salt, solvate, or hydrate thereof, is capsaicin. In one feature, the TrpV1 partial agonist is a fatty acid. In one feature, the TrpV1 partial agonist is a polyunsaturated fatty acid (PUFA). In one feature, the PUFA is a n-3 PUFA. In one feature, the PUFA is a n-6 PUFA. In one feature, the TrpV1 partial agonist comprises DHA (docosahexaenoic acid), EPA (eicosapentaenoic acid), LNA (linolenic acid), LA (linoleic acid), or combinations thereof. In one feature, the TrpV1 partial agonist comprises a free fatty acid, ester, triglyceride, or combination thereof of DHA (docosahexaenoic acid), EPA (eicosapentaenoic acid), LNA (linolenic acid), or LA (linoleic acid).

An aspect described herein are compositions comprising: (i) a compound of structural Formula (I):

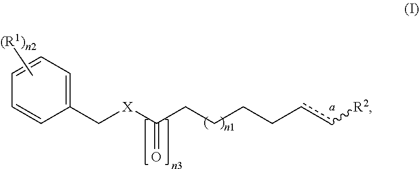

or an isotopic variant thereof; or a metabolite, pharmaceutically acceptable salt, solvate, or hydrate thereof,
   wherein: [symbol] is a single bond or double bond;

n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
(ii) a transient receptor potential cation channel subfamily V member 1 (TrpV1) antagonist; and
(iii) at least one excipient. In one feature, the compound of structural Formula (I), or an isotopic variant thereof; or a metabolite, pharmaceutically acceptable salt, solvate, or hydrate thereof, is capsaicin. In one feature, the TrpV1 antagonist is a fatty acid. In one feature, the TrpV1 antagonist is a polyunsaturated fatty acid (PUFA). In one feature, the PUFA is a n-3 PUFA. In one feature, the PUFA is a n-6 PUFA. In one feature, the TrpV1 antagonist comprises DHA (docosahexaenoic acid), EPA (eicosapentaenoic acid), LNA (linolenic acid), LA (linoleic acid), petroselinic acid, oleic acid, or combinations thereof. In one feature, the TrpV1 antagonist comprises a free fatty acid, ester, triglyceride, or combination thereof of DHA (docosahexaenoic acid), EPA (eicosapentaenoic acid), LNA (linolenic acid), LA (linoleic acid), petroselinic acid, or oleic acid. In one feature, the TrpV1 antagonist is a synthetic TrpV1 antagonist. In one feature, the TrpV1 antagonist does not cause hyperthermia. In one feature, the TrpV1 antagonist is

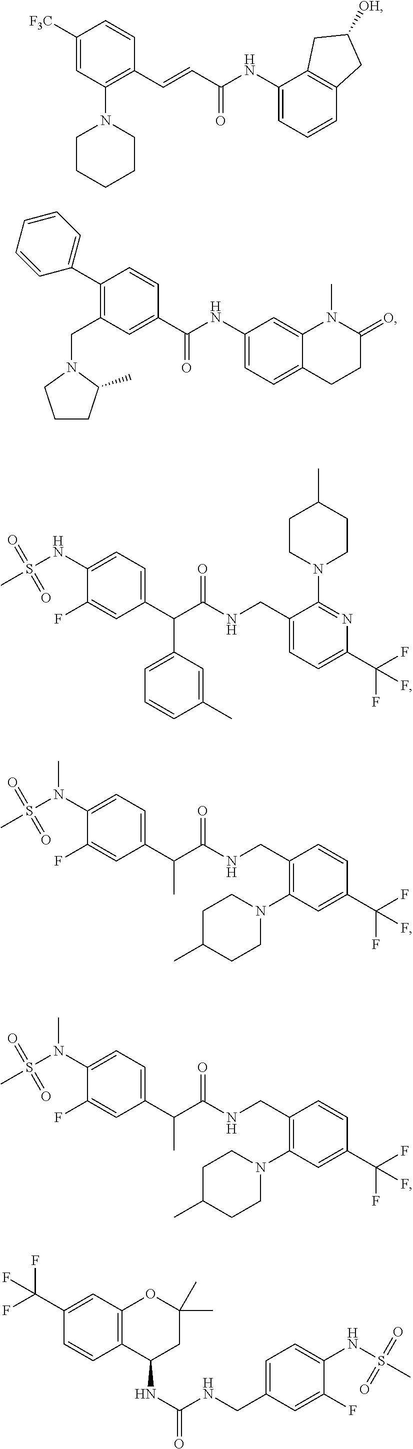

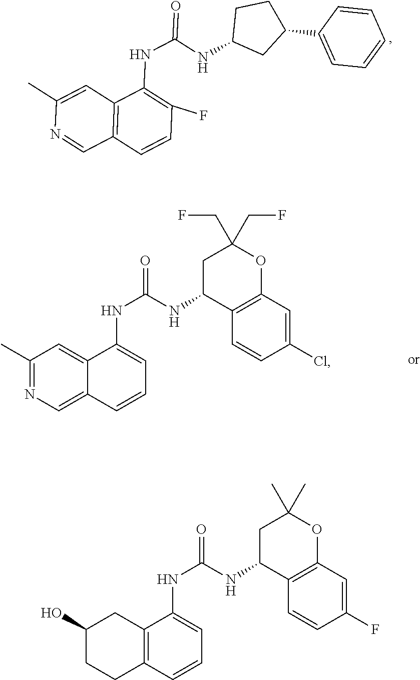 [SMILES: Cc1cc2c(NC(=O)N[C@@H]3CC[C@H](c4ccccc4)C3)c(F)ccc2cn1 ; Cc1cc2c(NC(=O)N[C@@H]3CC(CF)(CF)Oc4cc(Cl)ccc43)cccc2cn1 ; "or" ; CC1(C)C[C@@H](NC(=O)Nc2cccc3c2C[C@H](O)CC3)c2ccc(F)cc2O1]

or an isotopic variant thereof; or a metabolite, pharmaceutically acceptable salt, solvate, or hydrate thereof.

An aspect described herein are compositions comprising: (i) a compound of structural Formula (I):

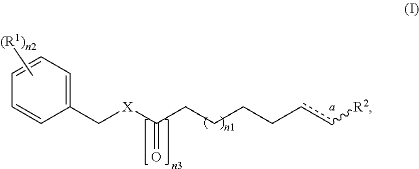

or an isotopic variant thereof; or a metabolite, pharmaceutically acceptable salt, solvate, or hydrate thereof,
wherein:
[symbol] is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
(ii) a transient receptor potential cation channel subfamily V member 1 (TrpV1) inverse agonist; and
(iii) at least one excipient. In one feature, the compound of structural Formula (I), or an isotopic variant thereof; or a metabolite, pharmaceutically acceptable salt, solvate, or hydrate thereof, is capsaicin. In one feature, the TrpV1 inverse agonist is a fatty acid. In one feature, the TrpV1 inverse agonist is a polyunsaturated fatty acid (PUFA). In one feature, the TrpV1 inverse agonist is anandamide or derivative thereof.

### Incorporation by Reference

All publications, patents, and patent applications mentioned in this specification are herein incorporated by reference to the same extent as if each individual publication, patent, or patent application was specifically and individually indicated to be incorporated by reference.

### DETAILED DESCRIPTION

Provided herein are, for example, compositions for treating and/or preventing a capsaicin-responsive disease or disorder. Also provided herein are, for example, methods of treating and/or preventing a capsaicin-responsive disease or disorder.

### Definitions

The abbreviations used herein have their conventional meaning within the chemical and biological arts. The chemical structures and formulae set forth herein are constructed according to the standard rules of chemical valency known in the chemical arts.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals, having the number of carbon atoms designated (i.e., C₁-C₁₀ means one to ten carbons). Alkyl is an uncyclized chain Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═32 N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N S, Si, or P). A heteroalkyl moiety may include four optionally different heteroatoms (e.g., O, N S, Si, or P). A heteroalkyl moiety may include five optionally different heteroatoms (e.g., O, N S, Si, or P). A heteroalkyl moiety may include up to eight optionally different heteroatoms (e.g., O, N S, Si, or P).

Each of the above terms (e.g., “alkyl,” “heteroalkyl,” etc.) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′—S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2 m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, -NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂T′—NR—C(NR′R″R′″)═NR″″, —NR—C(NR′R″)═NR′″, —S(O)R′, —S(O)₂R′—S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

A “substituent group,” as used herein, means a group selected from the following moieties:
(A) oxo, halogen, —CF₃, —CN, —OH, —NH₂, —COOH, —CONH₂, —NO2, —SH, —SO₃H, —SO₄H, —SO₂NH₂, —NHNH₂, —ONH₂, —NHC═(O)NHNH₂, —NHC═(O) NH₂, —NHSO₂H, —NHC═ (O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, heteroaryl, substituted with at least one substituent selected from:
   (i) oxo, halogen, —CF₃, —CN, —OH, —NH₂, —COOH, —CONH₂, —NO2, —SH, —SO₃H, —SO₄H, —SO₂NH₂, —NHNH₂, —ONH₂, —NHC═(O)NHNH₂, —NHC═(O) NH₂, —NHSO₂H, —NHC═ (O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, heteroaryl, substituted with at least one substituent selected from:
      (a) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═ (O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, heteroaryl, substituted with at least one substituent selected from: oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═ (O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl.

A “size-limited substituent” or “ size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₆-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “ lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

Certain compounds disclosed herein possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The presently disclosed compounds include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope hereof

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope hereof

The compounds of the present disclosure may also contain unnatural proportions of atomic isotopes (i.e., isotopic variants) at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents. In embodiments, compounds of the present disclosure contain both basic and acidic functionalities that allow the compounds to be converted into either base or acid addition salts. The neutral forms of the compounds may be regenerated by contacting the salt with a base or acid and isolating the parent compound in a conventional manner. The parent form of the compounds differs from the various salt forms in certain physical properties, such as solubility in polar solvents, but, unless specifically indicated, the salts disclosed herein are equivalent to the parent form of the compound for the purposes of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of a compound to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway. In some embodiments contacting includes allowing a compound described herein to interact with mitochondrial complex I.

As defined herein, the term “inhibition,” “inhibit,” “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g., an inhibitor binds to a protein that activates the target protein, thereby preventing target protein activation).

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist. An antagonist prevents, reduces, inhibits, or neutralizes the activity of an agonist, and an antagonist can also prevent, inhibit, or reduce constitutive activity of a target, e.g., a target receptor, even where there is no identified agonist. In embodiments, inhibitors are molecules that decrease, block, prevent, delay activation, inactivate, desensitize, or down-regulate, e.g., a gene, protein, ligand, receptor, or cell. An inhibitor may also be defined as a molecule that reduces, blocks, or inactivates a constitutive activity. An “antagonist” is a molecule that opposes the action(s) of an agonist.

The term “preparation” is intended to include the formulation of the active compound with encapsulating material as a carrier providing a capsule in which the active component with or without other carriers, is surrounded by a carrier, which is thus in association with it Similarly, cachets and lozenges are included. Tablets, powders, capsules, pills, cachets, and lozenges can be used as solid dosage forms suitable for oral administration.

The terms “disease” or “condition” refer to a state of being or health status of a patient or subject capable of being treated with the compounds or methods provided herein. The disease may be an autoimmune disease. The disease may be an inflammatory disease.

The disease may be an infectious disease. The infectious disease may be caused by a bacteria, viruses, parasites, or fungi. In some embodiments, the infectious disease is caused by Abelson leukemia virus, Abelson murine leukemia virus, Abelson's virus, Acute laryngotracheobronchitis virus, Adelaide River virus, Adeno associated virus group, Adenovirus, African horse sickness virus, African swine fever virus, AIDS virus, Aleutian mink disease parvovirus, Alpharetrovirus, Alphavirus, ALV related virus, Amapari virus, Aphthovirus, Aquareovirus, Arbovirus, Arbovirus C, arbovirus group A, arbovirus group B, Arenavirus group, Argentine hemorrhagic fever virus, Argentine hemorrhagic fever virus, Arterivirus, Astrovirus, Ateline herpesvirus group, Aujezky's disease virus, Aura virus, Ausduk disease virus, Australian bat lyssavirus, Aviadenovirus, avian erythroblastosis virus, avian infectious bronchitis virus, avian leukemia virus, avian leukosis virus, avian lymphomatosis virus, avian myeloblastosis virus, avian paramyxovirus, avian pneumoencephalitis virus, avian reticuloendotheliosis virus, avian sarcoma virus, avian type C retrovirus group, Avihepadnavirus, Avipoxvirus, B virus, B19 virus, Babanki virus, baboon herpesvirus, baculovirus, Barmah Forest virus, Bebaru virus, Berrimah virus, Betaretrovirus, Birnavirus, Bittner virus, BK virus, Black Creek Canal virus, bluetongue virus, Bolivian hemorrhagic fever virus, Boma disease virus, border disease of sheep virus, borna virus, bovine alphaherpesvirus 1, bovine alphaherpesvirus 2, bovine coronavirus, bovine ephemeral fever virus, bovine immunodeficiency virus, bovine leukemia virus, bovine leukosis virus, bovine mammillitis virus, bovine papillomavirus, bovine papular stomatitis virus, bovine parvovirus, bovine syncytial virus, bovine type C oncovirus, bovine viral diarrhea virus, Buggy Creek virus, bullet shaped virus group, Bunyamwera virus supergroup, Bunyavirus, Burkitt's lymphoma virus, Bwamba Fever, CA virus, Calicivirus, California encephalitis virus, camelpox virus, canarypox virus, canid herpesvirus, canine coronavirus, canine distemper virus, canine herpesvirus, canine minute virus, canine parvovirus, Cano Delgadito virus, caprine arthritis virus, caprine encephalitis virus, Caprine Herpes Virus, Capripox virus, Cardiovirus, caviid herpesvirus 1, Cercopithecid herpesvirus 1, cercopithecine herpesvirus 1, Cercopithecine herpesvirus 2, Chandipura virus, Changuinola virus, channel catfish virus, Charleville virus, chickenpox virus, Chikungunya virus, chimpanzee herpesvirus, chub reovirus, chum salmon virus, Cocal virus, Coho salmon reovirus, coital exanthema virus, Colorado tick fever virus, Coltivirus, Columbia SK virus, common cold virus, contagious eethyma virus, contagious pustular dermatitis virus, Coronavirus, Corriparta virus, coryza virus, cowpox virus, coxsackie virus, CPV (cytoplasmic polyhedrosis virus), cricket paralysis virus, Crimean-Congo hemorrhagic fever virus, croup associated virus, Cryptovirus, Cypovirus, Cytomegalovirus, cytomegalovirus group, cytoplasmic polyhedrosis virus, deer papillomavirus, deltaretrovirus, dengue virus, Densovirus, Dependovirus, Dhori virus, diploma virus, Drosophila C virus, duck hepatitis B virus, duck hepatitis virus 1, duck hepatitis virus 2, duovirus, Duvenhage virus, Deformed wing virus DWV, eastern equine encephalitis virus, eastern equine encephalomyelitis virus, EB virus, Ebola virus, Ebola-like virus, echo virus, echovirus, echovirus 10, echovirus 28, echovirus 9, ectromelia virus, EEE virus, EIA virus, EIA virus, encephalitis virus, encephalomyocarditis group virus, encephalomyocarditis virus, Enterovirus, enzyme elevating virus, enzyme elevating virus (LDH), epidemic hemorrhagic fever virus, epizootic hemorrhagic disease virus, Epstein-Barr virus, equid alphaherpesvirus 1, equid alphaherpesvirus 4, equid herpesvirus 2, equine abortion virus, equine arteritis virus, equine encephalosis virus, equine infectious anemia virus, equine morbillivirus, equine rhinopneumonitis virus, equine rhinovirus, Eubenangu virus, European elk papillomavirus, European swine fever virus, Everglades virus, Eyach virus, felid herpesvirus 1, feline calicivirus, feline fibrosarcoma virus, feline herpesvirus, feline immunodeficiency virus, feline infectious peritonitis virus, feline leukemia/sarcoma virus, feline leukemia virus, feline panleukopenia virus, feline parvovirus, feline sarcoma virus, feline syncytial virus, Filovirus, Flanders virus, Flavivirus, foot and mouth disease virus, Fort Morgan virus, Four Corners hantavirus, fowl adenovirus 1, fowlpox virus, Friend virus, Gammaretrovirus, GB hepatitis virus, GB virus, German measles virus, Getah virus, gibbon ape leukemia virus, glandular fever virus, goatpox virus, golden shinner virus, Gonometa virus, goose parvovirus, granulosis virus, Gross' virus, ground squirrel hepatitis B virus, group A arbovirus, Guanarito virus, guinea pig cytomegalovirus, guinea pig type C virus, Hantaan virus, Hantavirus, hard clam reovirus, hare fibroma virus, HCMV (human cytomegalovirus), hemadsorption virus 2, hemagglutinating virus of Japan, hemorrhagic fever virus, hendra virus, Henipaviruses, Hepadnavirus, hepatitis A virus, hepatitis B virus group, hepatitis C virus, hepatitis D virus, hepatitis delta virus, hepatitis E virus, hepatitis F virus, hepatitis G virus, hepatitis nonA nonB virus, hepatitis virus, hepatitis virus (nonhuman), hepatoencephalomyelitis reovirus 3, Hepatovirus, heron hepatitis B virus, herpes B virus, herpes simplex virus, herpes simplex virus 1, herpes simplex virus 2, herpesvirus, herpesvirus 7, Herpesvirus ateles, Herpesvirus hominis, Herpesvirus infection, Herpesvirus saimiri, Herpesvirus suis, Herpesvirus varicellae, Highlands J virus, Hirame rhabdovirus, hog cholera virus, human adenovirus 2, human alphaherpesvirus 1, human alphaherpesvirus 2, human alphaherpesvirus 3, human B lymphotropic virus, human betaherpesvirus 5, human coronavirus, human cytomegalovirus group, human foamy virus, human gammaherpesvirus 4, human gammaherpesvirus 6, human hepatitis A virus, human herpesvirus 1 group, human herpesvirus 2 group, human herpesvirus 3 group, human herpesvirus 4 group, human herpesvirus 6, human herpesvirus 8, human immunodeficiency virus, human immodeficiency virus 1, human immunodeficiency virus 2, human papillomavirus, human T cell leukemia virus, human T cell leukemia virus I, human T cell leukemia virus II, human T cell leukemia virus III, human T cell lymphoma virus I, human T cell lymphoma virus II, human T cell lymphotropic virus type 1, human T cell lymphotropic virus type 2, human T lymphotropic virus I, human T lymphotropic virus II, human T lymphotropic virus III, Ichnovirus, infantile gastroenteritis virus, infectious bovine rhinotracheitis virus, infectious haematopoietic necrosis virus, infectious pancreatic necrosis virus, influenza virus A, influenza virus B, influenza virus C, influenza virus D, influenza virus pr8, insect iridescent virus, insect virus, iridovirus, Japanese B virus, Japanese encephalitis virus, JC virus, Junin virus, Kaposi's sarcoma-associated herpesvirus, Kemerovo virus, Kilham's rat virus, Klamath virus, Kolongo virus, Korean hemorrhagic fever virus, kumba virus, Kysanur forest disease virus, Kyzylagach virus, La Crosse virus, lactic dehydrogenase elevating virus, lactic dehydrogenase virus, Lagos bat virus, Langur virus, lapine parvovirus, Lassa fever virus, Lassa virus, latent rat virus, LCM virus, Leaky virus, Lentivirus, Leporipoxvirus, leukemia virus, leukovirus, lumpy skin disease virus, lymphadenopathy associated virus, Lymphocryptovirus, lymphocytic choriomeningitis virus, lymphoproliferative virus group, Machupo virus, mad itch virus, mammalian type B oncovirus group, mammalian type B retroviruses, mammalian type C retrovirus group, mammalian type D retroviruses, mammary tumor virus, Mapuera virus, Marburg virus, Marburg-like virus, Mason Pfizer monkey virus, Mastadenovirus, Mayaro virus, ME virus, measles virus, Menangle virus, Mengo virus, Mengovirus, Middelburg virus, milkers nodule virus, mink enteritis virus, minute virus of mice, MLV related virus, MM virus, Mokola virus, Molluscipoxvirus, Molluscum contagiosum virus, monkey B virus, monkeypox virus, Mononegavirales, Morbillivirus, Mount Elgon bat virus, mouse cytomegalovirus, mouse encephalomyelitis virus, mouse hepatitis virus, mouse K virus, mouse leukemia virus, mouse mammary tumor virus, mouse minute virus, mouse pneumonia virus, mouse poliomyelitis virus, mouse polyomavirus, mouse sarcoma virus, mousepox virus, Mozambique virus, Mucambo virus, mucosal disease virus, mumps virus, murid betaherpesvirus 1, murid cytomegalovirus 2, murine cytomegalovirus group, murine encephalomyelitis virus, murine hepatitis virus, murine leukemia virus, murine nodule inducing virus, murine polyomavirus, murine sarcoma virus, Muromegalovirus, Murray Valley encephalitis virus, myxoma virus, Myxovirus, Myxovirus multiforme, Myxovirus parotitidis, Nairobi sheep disease virus, Nairovirus, Nanirnavirus, Nariva virus, Ndumo virus, Neethling virus, Nelson Bay virus, neurotropic virus, New World Arenavirus, newborn pneumonitis virus, Newcastle disease virus, Nipah virus, noncytopathogenic virus, Norwalk virus, nuclear polyhedrosis virus (NPV), nipple neck virus, O'nyong'nyong virus, Ockelbo virus, oncogenic virus, oncogenic viruslike particle, oncornavirus, Orbivirus, Orf virus, Oropouche virus, Orthohepadnavirus, Orthomyxovirus, Orthopoxvirus, Orthoreovirus, Orungo, ovine papillomavirus, ovine catarrhal fever virus, owl monkey herpesvirus, Palyam virus, Papillomavirus, Papillomavirus sylvilagi, Papovavirus, parainfluenza virus, parainfluenza virus type 1, parainfluenza virus type 2, parainfluenza virus type 3, parainfluenza virus type 4, Paramyxovirus, Parapoxvirus, paravaccinia virus, Parvovirus, Parvovirus B19, parvovirus group, Pe stivirus, Phlebovirus, phocine distemper virus, Picodnavirus, Picornavirus, pig cytomegalovirus-pigeonpox virus, Piry virus, Pixuna virus, pneumonia virus of mice, Pneumovirus, poliomyelitis virus, poliovirus, Polydnavirus, polyhedral virus, polyoma virus, Polyomavirus, Polyomavirus bovis, Polyomavirus cercopitheci, Polyomavirus hominis 2, Polyomavirus maccacae 1, Polyomavirus muris 1, Polyomavirus muris 2, Polyomavirus papionis 1, Polyomavirus papionis 2, Polyomavirus sylvilagi, Pongine herpesvirus 1, porcine epidemic diarrhea virus, porcine hemagglutinating encephalomyelitis virus, porcine parvovirus, porcine transmissible gastroenteritis virus, porcine type C virus, pox virus, poxvirus, poxvirus variolae, Prospect Hill virus, Provirus, pseudocowpox virus, pseudorabies virus, psittacinepox virus, quailpox virus, rabbit fibroma virus, rabbit kidney vaculolating virus, rabbit papillomavirus, rabies virus, raccoon parvovirus, raccoonpox virus, Ranikhet virus, rat cytomegalovirus, rat parvovirus, rat virus, Rauscher's virus, recombinant vaccinia virus, recombinant virus, reovirus, reovirus 1, reovirus 2, reovirus 3, reptilian type C virus, respiratory infection virus, respiratory syncytial virus, respiratory virus, reticuloendotheliosis virus, Rhabdovirus, Rhabdovirus carpia, Rhadinovirus, Rhinovirus, Rhizidiovirus, Rift Valley fever virus, Riley's virus, rinderpest virus, RNA tumor virus, Ross River virus, Rotavirus, rougeole virus, Rous sarcoma virus, rubella virus, rubeola virus, Rubivirus, Russian autumn encephalitis virus, SA 11 simian virus, SA2 virus, Sabia virus, Sagiyama virus, Saimirine herpesvirus 1, salivary gland virus, sandfly fever virus group, Sandjimba virus, SARS virus, SDAV (sialodacryoadenitis virus), sealpox virus, Semliki Forest Virus, Seoul virus, sheeppox virus, Shope fibroma virus, Shope papilloma virus, simian foamy virus, simian hepatitis A virus, simian human immunodeficiency virus, simian immunodeficiency virus, simian parainfluenza virus, simian T cell lymphotrophic virus, simian virus, simian virus 40, Simplexvirus, Sin Nombre virus, Sindbis virus, smallpox virus, South American hemorrhagic fever viruses, sparrowpox virus, Spumavirus, squirrel fibroma virus, squirrel monkey retrovirus, SSV 1 virus group, STLV (simian T lymphotropic virus) type I, STLV (simian T lymphotropic virus) type II, STLV (simian T lymphotropic virus) type III, stomatitis papulosa virus, submaxillary virus, suid alphaherpesvirus 1, suid herpesvirus 2, Suipoxvirus, swamp fever virus, swinepox virus, Swiss mouse leukemia virus, TAC virus, Tacaribe complex virus, Tacaribe virus, Tanapox virus, Taterapox virus, Tench reovirus, Theiler's encephalomyelitis virus, Theiler's virus, Thogoto virus, Thottapalayam virus, Tick borne encephalitis virus, Tioman virus, Togavirus, Torovirus, tumor virus, Tupaia virus, turkey rhinotracheitis virus, turkeypox virus, type C retroviruses, type D oncovirus, type D retrovirus group, ulcerative disease rhabdovirus, Una virus, Uukuniemi virus group, vaccinia virus, vacuolating virus, varicella zoster virus, Varicellovirus, Varicola virus, variola major virus, variola virus, Vasin Gishu disease virus, VEE virus, Venezuelan equine encephalitis virus, Venezuelan equine encephalomyelitis virus, Venezuelan hemorrhagic fever virus, vesicular stomatitis virus, Vesiculovirus, Vilyuisk virus, viper retrovirus, viral haemorrhagic septicemia virus, Visna Maedi virus, Visna virus, volepox virus, VSV (vesicular stomatitis virus), Wallal virus, Warrego virus, wart virus, WEE virus, West Nile virus, western equine encephalitis virus, western equine encephalomyelitis virus, Whataroa virus, Winter Vomiting Virus, woodchuck hepatitis B virus, woolly monkey sarcoma virus, wound tumor virus, WRSV virus, Yaba monkey tumor virus, Yaba virus, Yatapoxvirus, yellow fever virus, or the Yug Bogdanovac virus. In some embodiments, the infectious disease is caused by the coronavirus.

The disease may be a cancer. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including MDS, AML, ALL, ATLL and CML), or multiple myeloma. As used herein, the term “cancer” refers to all types of cancer, neoplasm or malignant tumors found in mammals (e.g. humans), including leukemia, carcinomas and sarcomas. Exemplary cancers that may be treated with a compound or method provided herein include brain cancer, glioma, glioblastoma, neuroblastoma, prostate cancer, colorectal cancer, pancreatic cancer, cervical cancer, gastric cancer, ovarian cancer, lung cancer, and cancer of the head. Exemplary cancers that may be treated with a compound or method provided herein include cancer of the thyroid, endocrine system, brain, breast, cervix, colon, head & neck, liver, kidney, lung, non-small cell lung, melanoma, mesothelioma, ovary, sarcoma, stomach, uterus, Medulloblastoma, colorectal cancer, pancreatic cancer. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the term “autoimmune disease” refers to a disease or condition in which a subject's immune system has an aberrant immune response against a substance that does not normally elicit an immune response in a healthy subject. Examples of autoimmune diseases that may be treated with a compound, pharmaceutical composition, or method described herein include Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg—Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch—Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA). In some embodiments, the autoimmune disease is not asthma.

As used herein, the term “inflammatory disease” refers to a disease or condition characterized by aberrant inflammation (e.g. an increased level of inflammation compared to a control such as a healthy person not suffering from a disease). Examples of inflammatory diseases include traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo,asthma, asthma, allergic asthma, acne vulgaris, celiac disease, chronic prostatitis, inflammatory bowel disease, pelvic inflammatory disease, reperfusion injury, sarcoidosis, transplant rejection, interstitial cystitis, atherosclerosis, and atopic dermatitis.

The terms “treating” or “treatment” refer to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

“Treating” and “treatment” as used herein include prophylactic treatment. Treatment methods include administering to a subject a therapeutically effective amount of a compound described herein. The administering step may consist of a single administration or may include a series of administrations. The length of the treatment period depends on a variety of factors, such as the severity of the condition, the age of the patient, the concentration of the compound, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and duration sufficient to treat the patient.

The terms “prevent,” “preventing,” and “prevention” refer to a decrease in the occurrence of disease symptoms in a patient. The preventing or prevention may be complete (no detectable symptoms) or partial, such that fewer symptoms are observed than would likely occur absent treatment. In embodiments, prevent refers to slowing the progression of the disease, disorder or condition or inhibiting progression thereof to a harmful or otherwise undesired state.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals including, but not limited to, fish and birds.F″effec In some embodiments, a patient is human.

Treatment, as referred to herein, also refers to the systemic delivery of the compounds disclosed herein to any type of plant, including trees, shrubs, flowering plants, foliage plants, house plants, groundcover and grass, and agronomic plants (including crops of agronomic plants).

An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). The therapeutically effective amount can be ascertained by measuring relevant physiological effects, and it can be adjusted in connection with the dosing regimen and diagnostic analysis of the subject's condition, and the like. By way of example, measurement of the serum level of a compound of Formula (I) or a hydrate, solvate, tautomer, or pharmaceutically acceptable salt thereof (or, e.g., a metabolite thereof) at a particular time post-administration may be indicative of whether a therapeutically effective amount has been administered.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan. Adjusting the dose to achieve maximal therapeutic window efficacy or toxicity in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., chemotherapeutic agent). The compound of the disclosure can be administered alone or can be coadministered to the patient. Coadministration is meant to include simultaneous or sequential administration of the compound individually or in combination (more than one compound or agent). Thus, the preparations can also be combined, when desired, with other active substances (e.g., to reduce metabolic degradation). The compositions of the present disclosure can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present disclosure may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present disclosure can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present disclosure can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present disclosure into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present disclosure can also be delivered as nanoparticles.

By “co-administer” it is meant that a composition described herein is administered at the same time, just prior to, or just after the administration of one or more additional therapies. The compounds of the disclosure can be administered alone or can be coadministered to the patient. Coadministration is meant to include simultaneous or sequential administration of the compounds individually or in combination (more than one compound). The compositions of the present disclosure can be delivered transdermally, by a topical route, or formulated as applicator sticks, solutions, suspensions, emulsions, gels, creams, ointments, pastes, jellies, paints, powders, and aerosols.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present disclosure should be sufficient to affect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The compounds described herein can be used for treating an infectious disease. In some embodiments, the compounds described herein are used as an anti-coagulant. The compounds described herein can be used in combination with one another, with other active agents known to be useful in treating cancer (e.g., colon cancer), cardiovascular disease, metabolic disease, immune or inflammatory disease or disorder, or an infectious disease or disorder.

In some embodiments, co-administration includes administering one active agent within 0.5, 1, 2, 4, 6, 8, 10, 12, 16, 20, 24 hours, 2 days, 4 days, 1 week or 1 month of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another. In some embodiments, the compounds described herein may be combined with treatments for cancer or infections (e.g., fungal infections, bacterial infections, viral infections, etc.)

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

The phrase “in a sufficient amount to effect a change” means that there is a detectable difference between a level of an indicator measured before (e.g., a baseline level) and after administration of a particular therapy. Indicators include any objective parameter (e.g., serum concentration) or subjective parameter (e.g., a subject's feeling of well-being).

“Substantially pure” indicates that a component makes up greater than about 50% of the total content of the composition, and typically greater than about 60% of the total polypeptide content. More typically, “substantially pure” refers to compositions in which at least 75%, at least 85%, at least 90% or more of the total composition is the component of interest. In some cases, the polypeptide will make up greater than about 90%, or greater than about 95% of the total content of the composition (percentage in a weight per weight basis).

“Capsaicin” as used herein is also referred to as (6E)-N-[(4-Hydroxy-3-methoxyphenypmethyl]-8-methylnon-6-enamide, (E)-N-(4-Hydroxy-3-methoxybenzyl)-8-methylnon-6-enamide, 8-Methyl-N-vanillyl-trans-6-nonenamide, trans-8-Methyl-N-vanillylnon-6-enamide, (E)-Capsaicin, Capsicine, Capsicin, and/or CPS and has the chemical structure:

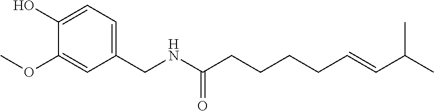 [SMILES: COc1cc(CNC(=O)CCCC/C=C/C(C)C)ccc1O]

In some embodiments, the compound of structural Formula (I), is capsaicin or an isotopic variant thereof; or a metabolite, pharmaceutically acceptable salt, solvate, or hydrate thereof.

### Compositions

In an aspect, provided herein is a composition, comprising:
(i) a compound of structural Formula (I):

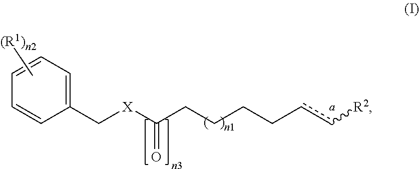

or an isotopic variant thereof; or a metabolite, pharmaceutically acceptable salt, solvate, or hydrate thereof,
   wherein:
[symbol] is a single bond or double bond;

n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
(ii) at least one additional therapeutic agent selected from:
   a fatty acid amide hydrolase (FAAH) receptor inhibitor, and
   a specialized pro-resolving mediator (SPM), or a combination thereof; and
(iii) at least one excipient.

In an aspect, provided herein is a composition, consisting essentially of:
(i) a compound of structural Formula (I):

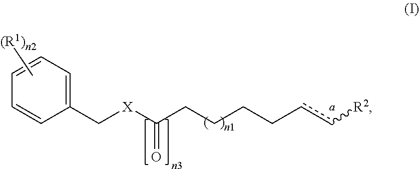

or an isotopic variant thereof; or a metabolite, pharmaceutically acceptable salt, solvate, or hydrate thereof,
   wherein: [symbol] is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
(ii) a transient receptor potential cation channel subfamily V member 1 (TrpV1) modulator; and
(iii) at least one excipient.

In an aspect, provided herein is a composition, consisting essentially of:
(i) capsaicin, or an isotopic variant thereof; or a metabolite, pharmaceutically acceptable salt, solvate, or hydrate thereof;
(ii) a transient receptor potential cation channel subfamily V member 1 (TrpV1) modulator; and
(iii) at least one excipient.

In an aspect, provided herein is a composition, consisting essentially of:
(i) capsaicin, or an isotopic variant thereof; or a metabolite, pharmaceutically acceptable salt, solvate, or hydrate thereof;
(ii) oleic acid; and
(iii) at least one excipient.

In an aspect, provided herein is a composition, comprising:
(i) a compound of structural Formula (I):

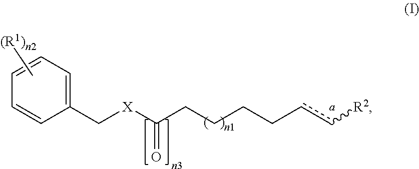

or an isotopic variant thereof; or a metabolite, pharmaceutically acceptable salt, solvate, or hydrate thereof,
   wherein: [symbol] is a single bond or double bond;
n1 and n2 are independently an integer from 0 to 2;
n3 is 0 or 1;
X is O, NH, or S;
R¹ is independently hydrogen, halogen, —OR^{1a}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
(ii) a transient receptor potential cation channel subfamily V member 1 (TrpV1) modulator; and
(iii) at least one excipient,
   wherein when the compound of structural Formula (I), or an isotopic variant thereof; or a metabolite, pharmaceutically acceptable salt, solvate, or hydrate thereof, is capsaicin, then the capsaicin is present in the composition in an amount of greater than about 40% (w/v).

In some embodiments, when the compound of structural Formula (I), or an isotopic variant thereof; or a metabolite, pharmaceutically acceptable salt, solvate, or hydrate thereof, is capsaicin, gingerols, piperines, or shogaols, then the capsaicin, gingerols, piperines, or shogaols are present in the composition in an amount of greater than about 40% (w/v). In some embodiments, when the compound of structural Formula (I), or an isotopic variant thereof; or a metabolite, pharmaceutically acceptable salt, solvate, or hydrate thereof, is capsaicin, guaiacol, eugenol, Zingerone, civamide, nonivamide, nuvanil, olvanil, NE-19550, NE-21610, or NE-28345, then the capsaicin, guaiacol, eugenol, Zingerone, civamide, nonivamide, nuvanil, olvanil, NE-19550, NE-21610, or NE-28345 are present in the composition in an amount of from about 40% (w/v) to about 50% (w/v).

In an aspect, provided herein is a composition, comprising:
(i) a compound of structural Formula (I):

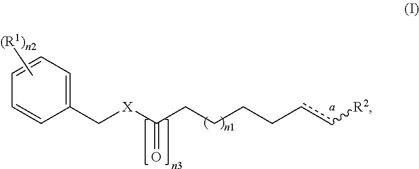

or an isotopic variant thereof; or a metabolite, pharmaceutically acceptable salt, solvate, or hydrate thereof,
   wherein:

[symbol] is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
(ii) a TrpV1 modulator; and
(iii) at least one excipient,
wherein the compound of Formula (I), isotopic variant thereof; or a metabolite, pharmaceutically acceptable salt, solvate, or hydrate thereof, is not capsaicin; or
wherein the TrpV1 modulator is not a fatty acid.

In some embodiments, the at least one TrpV1 modulator is a TrpV1 antagonist, a TrpV1 competitive agonist, or combination thereof

In some embodiments, the at least one TrpV1 modulator is a triglyceride or spilanthol.

In some embodiments, the at least one TrpV1 modulator is a fatty acid, triglyceride, or spilanthol.

In some embodiments, the fatty acid is a polyunsaturated fatty acid (PUFA).

In some embodiments, the PUFA is anandamide

In some embodiments, the fatty acid is an omega-9 fatty acid.

In some embodiments, the fatty acid is oleic acid or erucic acid.

In some embodiments, the at least one additional therapeutic agent is a FAAH receptor inhibitor.

In some embodiments, the FAAH receptor inhibitor is SSR411298.

In some embodiments, the at least one additional therapeutic agent is a FAAH inhibitor.

In some embodiments, the FAAH inhibitor is a flavonoid. Exemplary flavonoids include, but are not limited to, myricetin, quercetin, kaempferol, luteolin, and apigenin. In some embodiments, the FAAH inhibitor is kaempferol.

In some embodiments, the at least one additional therapeutic agent is an SPM.

In some embodiments, the SPM is maresin.

In some embodiments, the pharmaceutical composition is in a form for oral dosing or administration.

In some embodiments, the form is a suspension or a solution.

In some embodiments, the form is a solid dosage form.

In some embodiments, the form is a tablet or a capsule.

In some embodiments, the pharmaceutical composition is in a form for inhalation, nebulization, or nasal delivery.

In some embodiments, the pharmaceutical composition is provided as an aerosol. In some embodiments, the pharmaceutical composition is provided as an inhalable aerosol. In some embodiments, the pharmaceutical composition is in a form of an electronic delivery system. In some embodiments, the pharmaceutical composition is a vaping device. In some embodiments, the pharmaceutical composition is provided as an e-cigarette.

In some embodiments, the pharmaceutical composition is in a form for inhalation administration or dosing by vaporizer.

In some embodiments, the pharmaceutical composition is in a form for parenteral administration or dosing.

In some embodiments, the parenteral administration or dosing is injection, infusion, or implantation.

In some embodiments, the injection is intravenous, intramuscular, subcutaneous, or intradermal.

In some embodiments, the pharmaceutical composition is in a form for dosing or administration by suppository.

In some embodiments, the pharmaceutical composition is in a form for cutaneous or transdermal administration.

In some embodiments, the pharmaceutical composition is in a form for sublingual or buccal administration.

In some embodiments, the compound of Formula (I), or an isotopic variant thereof; or a metabolite, pharmaceutically acceptable salt, solvate, or hydrate thereof is present in the composition in an amount of greater than about 10% (w/v).

In some embodiments, the compound of Formula (I), or an isotopic variant thereof; or a metabolite, pharmaceutically acceptable salt, solvate, or hydrate thereof is present in the composition in an amount of greater than about 20% (w/v).

In some embodiments, the compound of Formula (I), or an isotopic variant thereof; or a metabolite, pharmaceutically acceptable salt, solvate, or hydrate thereof is present in the composition in an amount of greater than about 30% (w/v).

In some embodiments, the compound of Formula (I), or an isotopic variant thereof; or a metabolite, pharmaceutically acceptable salt, solvate, or hydrate thereof is present in the composition in an amount of greater than about 40% (w/v).

In some embodiments, the compound of Formula (I), or an isotopic variant thereof; or a metabolite, pharmaceutically acceptable salt, solvate, or hydrate thereof is present in the composition in an amount of from about 10% (w/v) to about 50% (w/v). In some embodiments, the compound of Formula (I), or an isotopic variant thereof; or a metabolite, pharmaceutically acceptable salt, solvate, or hydrate thereof is present in the composition in an amount of from about 15% (w/v) to about 50% (w/v). In some embodiments, the compound of Formula (I), or an isotopic variant thereof; or a metabolite, pharmaceutically acceptable salt, solvate, or hydrate thereof is present in the composition in an amount of from about 20% (w/v) to about 50% (w/v). In some embodiments, the compound of Formula (I), or an isotopic variant thereof; or a metabolite, pharmaceutically acceptable salt, solvate, or hydrate thereof is present in the composition in an amount of from about 25% (w/v) to about 50% (w/v). In some embodiments, the compound of Formula (I), or an isotopic variant thereof; or a metabolite, pharmaceutically acceptable salt, solvate, or hydrate thereof is present in the composition in an amount of from about 30% (w/v) to about 50% (w/v). In some embodiments, the compound of Formula (I), or an isotopic variant thereof; or a metabolite, pharmaceutically acceptable salt, solvate, or hydrate thereof is present in the composition in an amount of from about 35% (w/v) to about 50% (w/v). In some embodiments, the compound of Formula (I), or an isotopic variant thereof; or a metabolite, pharmaceutically acceptable salt, solvate, or hydrate thereof is present in the composition in an amount of from about 40% (w/v) to about 50% (w/v). In some embodiments, the compound of Formula (I), or an isotopic variant thereof; or a metabolite, pharmaceutically acceptable salt, solvate, or hydrate thereof is present in the composition in an amount of from about 45% (w/v) to about 50% (w/v). In some embodiments, the compound of Formula (I), or an isotopic variant thereof; or a metabolite, pharmaceutically acceptable salt, solvate, or hydrate thereof is present in the composition in an amount of about 10% (w/v). In some embodiments, the compound of Formula (I), or an isotopic variant thereof; or a metabolite, pharmaceutically acceptable salt, solvate, or hydrate thereof is present in the composition in an amount of about 15% (w/v). In some embodiments, the compound of Formula (I), or an isotopic variant thereof; or a metabolite, pharmaceutically acceptable salt, solvate, or hydrate thereof is present in the composition in an amount of about 20% (w/v). In some embodiments, the compound of Formula (I), or an isotopic variant thereof; or a metabolite, pharmaceutically acceptable salt, solvate, or hydrate thereof is present in the composition in an amount of about 25% (w/v). In some embodiments, the compound of Formula (I), or an isotopic variant thereof; or a metabolite, pharmaceutically acceptable salt, solvate, or hydrate thereof is present in the composition in an amount of about 30% (w/v). In some embodiments, the compound of Formula (I), or an isotopic variant thereof; or a metabolite, pharmaceutically acceptable salt, solvate, or hydrate thereof is present in the composition in an amount of about 35% (w/v). In some embodiments, the compound of Formula (I), or an isotopic variant thereof; or a metabolite, pharmaceutically acceptable salt, solvate, or hydrate thereof is present in the composition in an amount of about 40% (w/v). In some embodiments, the compound of Formula (I), or an isotopic variant thereof; or a metabolite, pharmaceutically acceptable salt, solvate, or hydrate thereof is present in the composition in an amount of about 45% (w/v). In some embodiments, the compound of Formula (I), or an isotopic variant thereof; or a metabolite, pharmaceutically acceptable salt, solvate, or hydrate thereof is present in the composition in an amount of about 50% (w/v).

In an aspect, provided herein is a composition, comprising:
(i) a compound of structural Formula (I):

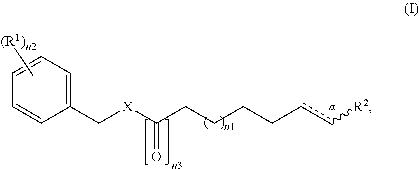

or an isotopic variant thereof; or a metabolite, pharmaceutically acceptable salt, solvate, or hydrate thereof,
   wherein:
[symbol] is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
(ii) a TrpV1 modulator; and
(iii) at least one excipient,
   wherein the composition is formulated for dosing or delivery by enteral or parenteral administration

In some embodiments, the composition is formulated as a liquid or a solid.

In some embodiments, the composition is formulated as a solid.

In some embodiments, the solid is a tablet, a capsule, a suppository, strip, or an implant.

In some embodiments, the tablet is a dissolvable tablet or a bilayer tablet.

In some embodiments, the strip is a dissolvable strip.

In some embodiments, the composition is formulated as a liquid.

In some embodiments, the liquid is a solution, a suspension, an emulsion, a syrup, an elixir, a tincture, an ointment, a soft gel, an enema, foam, cream, lotion, or a gel.

In some embodiments, the composition is formulated for dosing or delivery by enteral administration.

In some embodiments, the enteral administration is oral or rectal administration.

In some embodiments, the composition is formulated for dosing or delivery by parenteral administration.

In some embodiments, the parenteral administration is sublingual administration, buccal administration, or administration by injection, infusion, implantation, or inhalation.

In an aspect, provided herein is a composition, comprising:
(i) a compound of structural Formula (I):

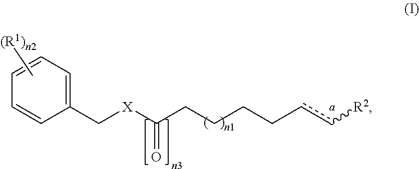

or an isotopic variant thereof; or a metabolite, pharmaceutically acceptable salt, solvate, or hydrate thereof,
   wherein:
[symbol] is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
   (ii) a TrpV1 modulator; and
   (iii) at least one excipient.

In some embodiments, the solid is a tablet, a capsule, a suppository, strip, or an implant.

In some embodiments, the tablet is a dissolvable tablet or a bilayer tablet.

In some embodiments, the strip is a dissolvable strip.

In an aspect, provided herein is a composition, comprising:
(i) a compound of structural Formula (I):

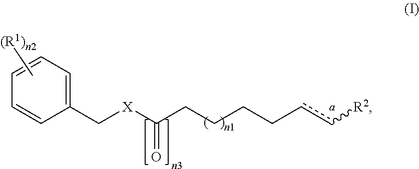

or an isotopic variant thereof; or a metabolite, pharmaceutically acceptable salt, solvate, or hydrate thereof,
   wherein:
[symbol] is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
(ii) a TrpV1 modulator; and
(iii) at least one excipient,
   wherein the compound of Formula (I), or an isotopic variant thereof; or a metabolite, pharmaceutically acceptable salt, solvate, or hydrate thereof, and the TrpV1 modulator are released sequentially in any order or have different release profiles. In some embodiments, the co-formulated compound of Formula (I), or an isotopic variant thereof; or a metabolite, pharmaceutically acceptable salt, solvate, or hydrate thereof, and the TrpV1 modulator are administered sequentially. In some embodiments, the co-formulated compound of Formula (I), or an isotopic variant thereof; or a metabolite, pharmaceutically acceptable salt, solvate, or hydrate thereof, and the TrpV1 modulator are administered sequentially in a bilayer tablet. In some embodiments, the co-formulated compound of Formula (I), or an isotopic variant thereof; or a metabolite, pharmaceutically acceptable salt, solvate, or hydrate thereof, and the TrpV1 modulator have different release profiles, for example the compound of Formula (I), or an isotopic variant thereof; or a metabolite, pharmaceutically acceptable salt, solvate, or hydrate thereof or the TrpV1 modulator is released immediately and the other is an extended release.

In some embodiments, the compound of Formula (I), or an isotopic variant thereof; or a metabolite, pharmaceutically acceptable salt, solvate, or hydrate thereof, is released before the at least one additional therapeutic agent.

In some embodiments, the compound of Formula (I), or an isotopic variant thereof; or a metabolite, pharmaceutically acceptable salt, solvate, or hydrate thereof, is released after the at least one additional therapeutic agent.

In some embodiments, the solid is a tablet, a capsule, a suppository, strip, or an implant.

In some embodiments, the tablet is a dissolvable tablet or a bilayer tablet.

In some embodiments, the strip is a dissolvable strip.

In some embodiments, the compound of Formula (I), or an isotopic variant thereof; or a metabolite, pharmaceutically acceptable salt, solvate, or hydrate thereof is present in the composition in an amount of greater than about 10% (w/v).

In some embodiments, the compound of Formula (I), or an isotopic variant thereof; or a metabolite, pharmaceutically acceptable salt, solvate, or hydrate thereof is present in the composition in an amount of greater than about 20% (w/v).

In some embodiments, the compound of Formula (I), or an isotopic variant thereof; or a metabolite, pharmaceutically acceptable salt, solvate, or hydrate thereof is present in the composition in an amount of greater than about 30% (w/v).

In some embodiments, the compound of Formula (I), or an isotopic variant thereof; or a metabolite, pharmaceutically acceptable salt, solvate, or hydrate thereof is present in the composition in an amount of greater than about 40% (w/v).

In some embodiments, the compound of Formula (I), or an isotopic variant thereof; or a metabolite, pharmaceutically acceptable salt, solvate, or hydrate thereof is present in the composition in an amount of from about 10% (w/v) to about 50% (w/v). In some embodiments, the compound of Formula (I), or an isotopic variant thereof; or a metabolite, pharmaceutically acceptable salt, solvate, or hydrate thereof is present in the composition in an amount of from about 20% (w/v) to about 50% (w/v). In some embodiments, the compound of Formula (I), or an isotopic variant thereof; or a metabolite, pharmaceutically acceptable salt, solvate, or hydrate thereof is present in the composition in an amount of from about 30% (w/v) to about 50% (w/v). In some embodiments, the compound of Formula (I), or an isotopic variant thereof; or a metabolite, pharmaceutically acceptable salt, solvate, or hydrate thereof is present in the composition in an amount of from about 40% (w/v) to about 50% (w/v).

In some embodiments, the composition is a modified-release dosage form, a sustained-release dosage form, or a controlled-release dosage form.

In some embodiments, the composition is in modified-release dosage form.

In some embodiments, the modified-release dosage form is a delayed-release dosage form, an extended release dosage form, or a targeted release dosage form.

In some embodiments, the ratio of TrpV1 modulator to a compound of Formula (I), or an isotopic variant thereof; or a metabolite, pharmaceutically acceptable salt, solvate, or hydrate thereof, is about 1:1.

In some embodiments, the ratio of FAAH receptor inhibitor to a compound of Formula (I), or an isotopic variant thereof; or a metabolite, pharmaceutically acceptable salt, solvate, or hydrate thereof, is about 1:1.

In some embodiments, the ratio of SPM to a compound of Formula (I), or an isotopic variant thereof; or a metabolite, pharmaceutically acceptable salt, solvate, or hydrate thereof, is about 1:1.

In some embodiments, the composition is formulated as a pharmaceutical composition.

In some embodiments, the at least one excipient is at least one pharmaceutically acceptable excipient.C

In some embodiments, the compound of structural Formula (I) has structural Formula (I-A):

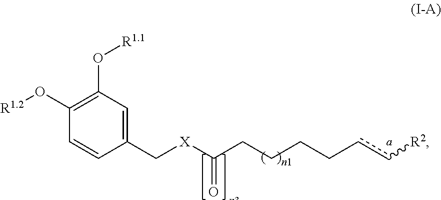

or an isotopic variant thereof; or a metabolite, pharmaceutically acceptable salt, solvate, or hydrate thereof,
   wherein:
R^{1.1} is hydrogen, halogen, —OR^{1.1A}, substituted or unsubstituted alkyl, or substituted or unsubstituted heteroalkyl;
R^{1.2} is hydrogen, halogen, —OR^{1.2A}, substituted or unsubstituted alkyl, or substituted or unsubstituted heteroalkyl; and
R^{1.1A} and R^{1.2A} are independently hydrogen, —CF₃, —CCl₃, —CBr3, —CI₃,—COOH, —CONH₂, substituted or unsubstituted alkyl, or substituted or unsubstituted heteroalkyl.

In some embodiments, R^{1.1} is CH₃; and R^{1.2} is OH.

a

In some embodiments, X is O. In some embodiments, X is NH. In some embodiments, [symbol] is a double bond. In some embodiments, [symbol] is a single bond. In some embodiments, n1 is 2. In some embodiments, n1 is 1. In some embodiments, n1 is 0. In some embodiments, R² is unsubstituted alkyl. In some embodiments, R² is —CH(CH₃)₂ or —CH₃.

In some embodiments, the compound is at least one of:

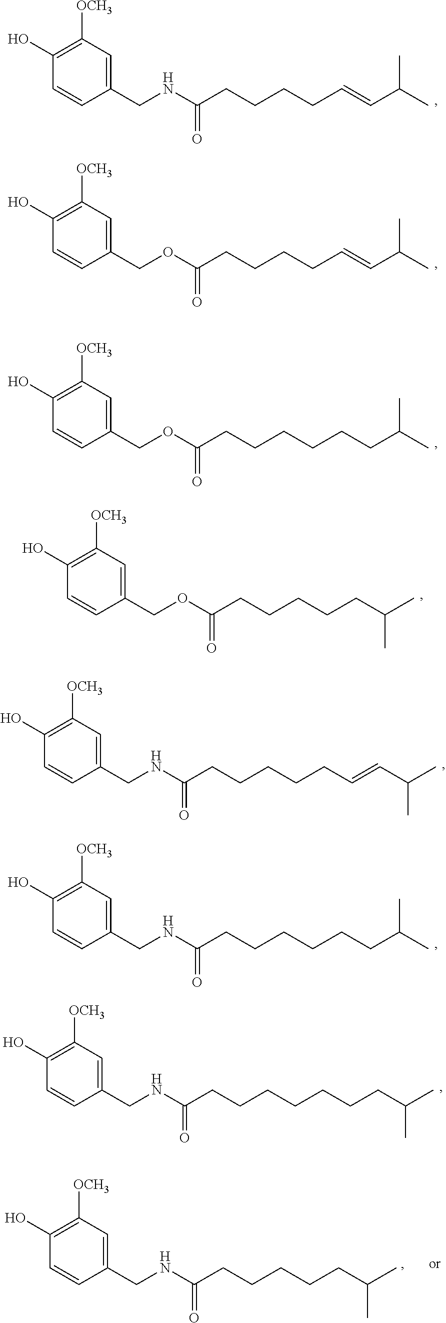

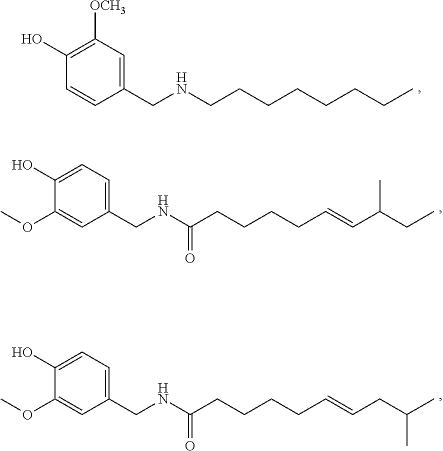

or an isotopic variant thereof; or a metabolite, pharmaceutically acceptable salt, solvate, or hydrate thereof.

In some embodiments, the compound is:

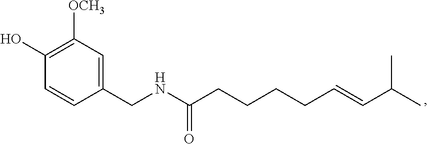 [SMILES: COc1cc(CNC(=O)CCCC/C=C/C(C)C)ccc1O]

or an isotopic variant thereof; or a metabolite, pharmaceutically acceptable salt, solvate, or hydrate thereof

In another aspect, provided herein is a supplement, comprising: (i) a compound of structural Formula (I):

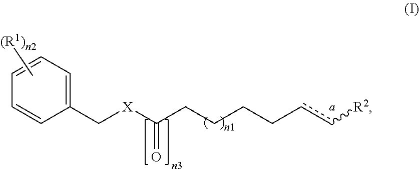

or an isotopic variant thereof; or a metabolite, pharmaceutically acceptable salt, solvate, or hydrate thereof,
   wherein:
[symbol] is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
(ii) at least one additional therapeutic agent selected from:
   a fatty acid amide hydrolase (FAAH) receptor inhibitor, and
   a specialized pro-resolving mediator (SPM), or a combination thereof; and
(iii) at least one excipient. In some embodiments, the supplement is in a form for oral dosing or administration (i.e., ingestion).

In some embodiments, the compound of structural Formula (I) has structural Formula (I-A):

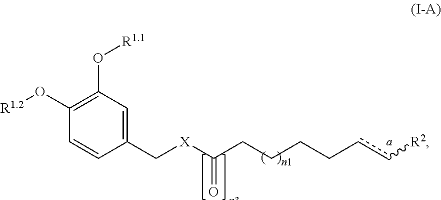

or an isotopic variant thereof; or a metabolite, pharmaceutically acceptable salt, solvate, or hydrate thereof,
   wherein:
R^{1.1} is hydrogen, halogen, —OR^{1.1A}, substituted or unsubstituted alkyl, or substituted or unsubstituted heteroalkyl;
R^{1.2} is hydrogen, halogen, —OR^{1.2A}, substituted or unsubstituted alkyl, or substituted or unsubstituted heteroalkyl;
R^{1.1A} and R^{1.2A} are independently hydrogen, —CF₃, —CCl₃, —CBr₃, —CI₃, —COOH, —CONH₂, substituted or unsubstituted alkyl, or substituted or unsubstituted heteroalkyl;

In some embodiments, R^{1.1} is CH₃; and R^{1.2} is OH.

In some embodiments, X is O. In some embodiments, X is NH. In some embodiments, [symbol] is a double bond. In some embodiments, [symbol] is a single bond. In some embodiments, n1 is 2. In some embodiments, n1 is 1. In some embodiments, n1 is 0. In some embodiments, R² is unsubstituted alkyl. In some embodiments, R² is —CH(CH₃)₂ or —CH₃.

In some embodiments, the compound is at least one of:

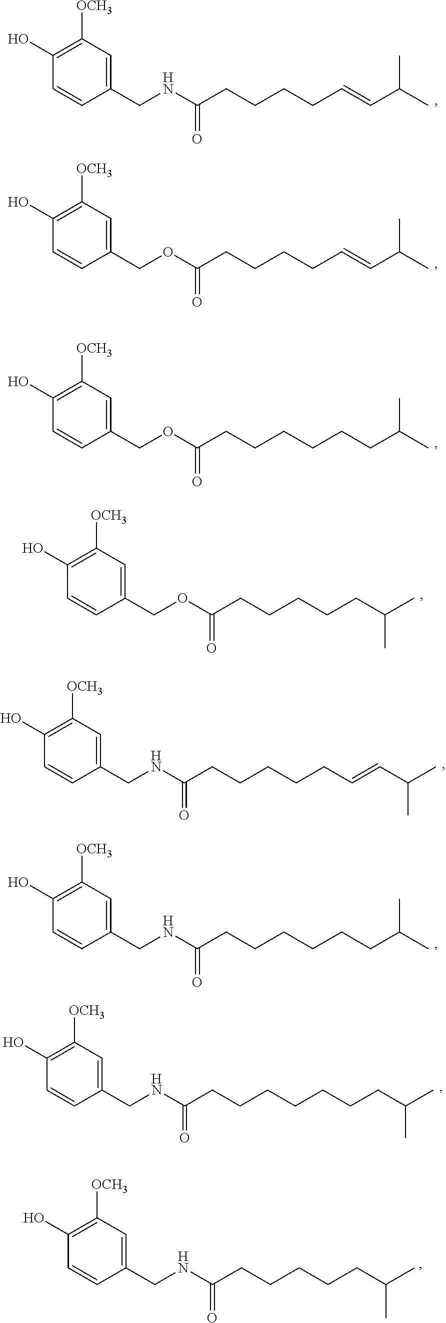

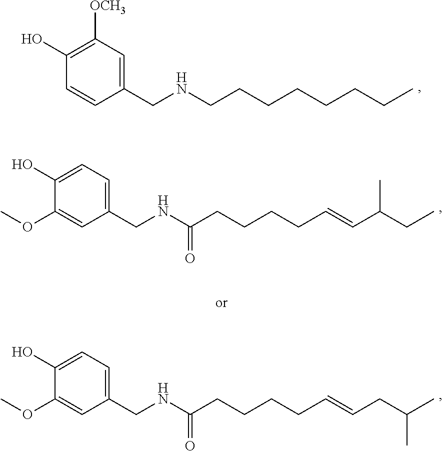

or an isotopic variant thereof; or a metabolite, pharmaceutically acceptable salt, solvate, or hydrate thereof.

In some embodiments, the compound is:

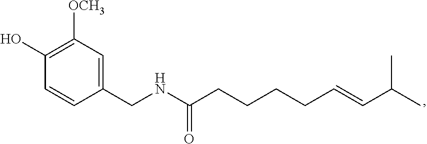 [SMILES: COc1cc(CNC(=O)CCCC/C=C/C(C)C)ccc1O]

or an isotopic variant thereof; or a metabolite, pharmaceutically acceptable salt, solvate, or hydrate thereof.

In some embodiments, the at least one additional therapeutic agent is a TrpV1 modulator. In some embodiments, the at least one TrpV1 modulator is a partial agonist, antagonist, an inverse agonist, or combinations thereof. In some embodiments, the at least one TrpV1 modulator is a TrpV1 antagonist, a TrpV1 competitive agonist, or combination thereof.

Exemplary TrpV1 modulators include, but are not limited to, DHA (docosahexaenoic acid), EPA (eicosapentaenoic acid), LNA (linolenic acid), LA (linoleic acid), petroselinic acid, or oleic acid, α-Linolenic acid, spilanthol, a FAHH receptor inhibitor, SSR411298, a maresin, a specialized proresolving mediators (SPM), anandamide, oleoylethanolamide, olvanil, arvanil, AM404, derivatives thereof, or combinations thereof.

In some embodiments, the at least one TrpV1 modulator is a partial agonist. Partial agonists comprise TrpV1 modulators that can activate the current through the ion channel In some embodiments, the partial agonist comprises a fatty acid. In some embodiments, the partial agonist comprises a polyunsaturated fatty acid (PUFA). In some embodiments, the partial agonist comprises a n-3 PUFA. In some embodiments, the partial agonist comprises a n-6 PUFA. In some embodiments, the partial agonist comprises DHA (docosahexaenoic acid), EPA (eicosapentaenoic acid), LNA (linolenic acid), LA (linoleic acid), or combinations thereof. In some embodiments, the partial agonist comprises a free fatty acid, ester, triglyceride, or combination thereof of DHA (docosahexaenoic acid), EPA (eicosapentaenoic acid), LNA (linolenic acid), or LA (linoleic acid).

In some embodiments, the at least one TrpV1 modulator is an antagonist. Antagonists comprise TrpV1 modulators that can inhibit the current evoked by an agonist. In some embodiments, a partial agonist acts as an antagonist since by displacing an agonist in the same binding site. In some embodiments, the antagonist comprises a fatty acid. In some embodiments, the antagonist comprises a polyunsaturated fatty acid (PUFA). In some embodiments, the antagonist comprises a n-3 PUFA. In some embodiments, the antagonist comprises a n-6 PUFA. In some embodiments, the antagonist comprises DHA (docosahexaenoic acid), EPA (eicosapentaenoic acid), LNA (linolenic acid), LA (linoleic acid), petroselinic acid, oleic acid, or combinations thereof. In some embodiments, the antagonist comprises a free fatty acid, ester, triglyceride, or combination thereof of DHA (docosahexaenoic acid), EPA (eicosapentaenoic acid), LNA (linolenic acid), LA (linoleic acid), petroselinic acid, or oleic acid.

In some embodiments, the antagonist is a synthetic TrpV1 antagonist. In some embodiments, the TrpV1 antagonist does not cause hyperthermia. In some embodiments, the TrpV1 antagonist do not affect H⁺ activation of the channel In some embodiments, the TrpV1 antagonist is

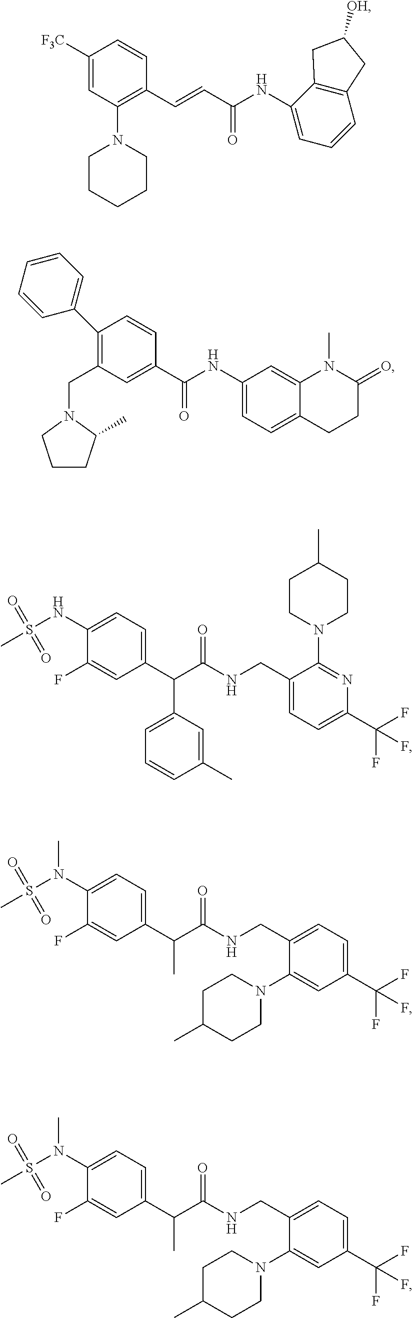 [SMILES: O=C(/C=C/c1ccc(C(F)(F)F)cc1N1CCCCC1)Nc1cccc2c1C[C@H](O)C2 ; C[C@@H]1CCCN1Cc1cc(C(=O)Nc2ccc3c(c2)N(C)C(=O)CC3)ccc1-c1ccccc1 ; Cc1cccc(C(C(=O)NCc2ccc(C(F)(F)F)nc2N2CCC(C)CC2)c2ccc(NS(C)(=O)=O)c(F)c2)c1 ; CC1CCN(c2cc(C(F)(F)F)ccc2CNC(=O)C(C)c2ccc(N(C)S(C)(=O)=O)c(F)c2)CC1 ; CC1CCN(c2cc(C(F)(F)F)ccc2CNC(=O)C(C)c2ccc(N(C)S(C)(=O)=O)c(F)c2)CC1]

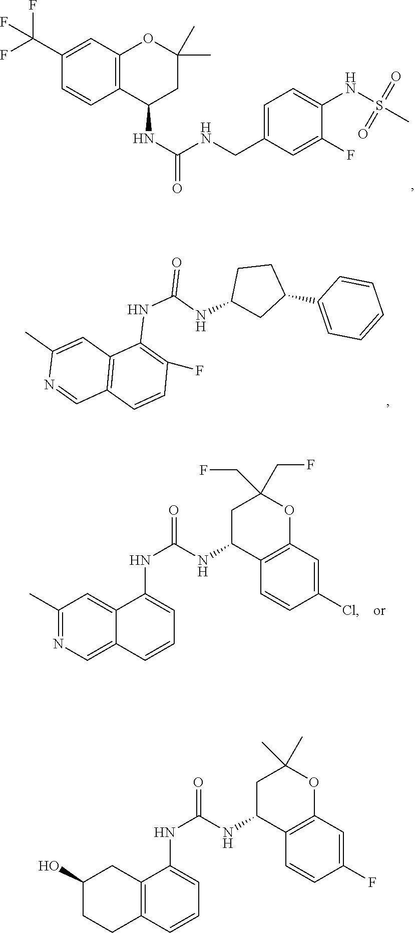

or an isotopic variant thereof; or a metabolite, pharmaceutically acceptable salt, solvate, or hydrate thereof.

In some embodiments, the at least one TrpV1 modulator is an inverse agonist. In some embodiments, the inverse agonist comprises a fatty acid. In some embodiments, the inverse agonist comprises a polyunsaturated fatty acid (PUFA). In some embodiments, the inverse agonist is anandamide. In some embodiments, the inverse agonist has a similar structure as anandamide. In some embodiments, the inverse agonist has a similar mechanism of action as anandamide Exemplary agents that have a similar structure, similar mechanism, or both include, but are not limited to, oleyethanolamide (OEA), olvanil, arvanil, and AM404.

In some embodiments, the at least one TrpV1 modulator is a fatty acid, triglyceride, or spilanthol.

In some embodiments, the fatty acid is a polyunsaturated fatty acid (PUFA).

In some embodiments, the PUFA is anandamide

In some embodiments, the fatty acid is an omega-9 fatty acid.

In some embodiments, the fatty acid is oleic acid or erucic acid.

In some embodiments, the at least one additional therapeutic agent is a FAAH receptor inhibitor.

In some embodiments, the FAAH receptor inhibitor is SSR411298.

In some embodiments, the at least one additional therapeutic agent is a FAAH inhibitor.

In some embodiments, the FAAH inhibitor is a flavonoid. Exemplary flavonoids include, but are not limited to, myricetin, quercetin, kaempferol, luteolin, and apigenin. In some embodiments, the FAAH inhibitor is kaempferol.

In some embodiments, the at least one additional therapeutic agent is an SPM.

In some embodiments, the SPM is maresin.

In some embodiments, the supplement is in a form for oral dosing or administration. In some embodiments, the form is a suspension or a solution. In some embodiments, the form is a solid dosage form. In some embodiments, the solid dosage form is a tablet or a capsule.

In some embodiments, the supplement is in a form for inhalation, nebulization, or nasal delivery.

In some embodiments, the pharmaceutical composition is provided as an aerosol. In some embodiments, the pharmaceutical composition is provided as an inhalable aerosol. In some embodiments, the pharmaceutical composition is in a form of an electronic delivery system. In some embodiments, the pharmaceutical composition is a vaping device. In some embodiments, the pharmaceutical composition is provided as an e-cigarette.

In some embodiments, the supplement is in a form for inhalation administration or dosing by vaporizer.

In some embodiments, the supplement is in a form for parenteral administration or dosing.

In some embodiments, the administration or dosing is parenteral injection.

In some embodiments, the injection is intravenous, intramuscular, subcutaneous, or intradermal.

In some embodiments, the supplement is in a form for dosing or administration by suppository.

In some embodiments, the supplement is in a form for cutaneous or transdermal administration.

In some embodiments, the supplement is in a form for sublingual or buccal administration.

In some embodiments, the compound of Formula (I), or an isotopic variant thereof; or a metabolite, pharmaceutically acceptable salt, solvate, or hydrate thereof is present in the composition in an amount of greater than about 10% (w/v).

In some embodiments, the compound of Formula (I), or an isotopic variant thereof; or a metabolite, pharmaceutically acceptable salt, solvate, or hydrate thereof is present in the composition in an amount of greater than about 20% (w/v).

In some embodiments, the compound of Formula (I), or an isotopic variant thereof; or a metabolite, pharmaceutically acceptable salt, solvate, or hydrate thereof is present in the composition in an amount of greater than about 30% (w/v).

In some embodiments, the compound of Formula (I), or an isotopic variant thereof; or a metabolite, pharmaceutically acceptable salt, solvate, or hydrate thereof is present in the composition in an amount of greater than about 40% (w/v).

In some embodiments, the compound of Formula (I), or an isotopic variant thereof; or a metabolite, pharmaceutically acceptable salt, solvate, or hydrate thereof is present in the composition in an amount of from about 10% (w/v) to about 50% (w/v). In some embodiments, the compound of Formula (I), or an isotopic variant thereof; or a metabolite, pharmaceutically acceptable salt, solvate, or hydrate thereof is present in the composition in an amount of from about 20% (w/v) to about 50% (w/v). In some embodiments, the compound of Formula (I), or an isotopic variant thereof; or a metabolite, pharmaceutically acceptable salt, solvate, or hydrate thereof is present in the composition in an amount of from about 30% (w/v) to about 50% (w/v). In some embodiments, the compound of Formula (I), or an isotopic variant thereof; or a metabolite, pharmaceutically acceptable salt, solvate, or hydrate thereof is present in the composition in an amount of from about 40% (w/v) to about 50% (w/v).

In some embodiments of the pharmaceutical compositions disclosed herein, the form is a suspension or a solution.

In some embodiments of the pharmaceutical compositions disclosed herein, the form is a tablet or a capsule.

In some embodiments of the pharmaceutical compositions disclosed herein, wherein the form is a tablet or a capsule, the tablet or capsule has an enteric coating.

In some embodiments of the pharmaceutical compositions disclosed herein, wherein the form is a tablet, the tablet is an osmotic floating tablet.

In some embodiments of the pharmaceutical compositions disclosed herein, wherein the form is a capsule, the capsule is a liquid-filled hard capsule.

In some embodiments of the pharmaceutical compositions disclosed herein, wherein the form is a capsule, the capsule is a soft gelatin capsule.

In some embodiments of the pharmaceutical compositions disclosed herein, the form is a modified-release dosage form. In some embodiments of the pharmaceutical compositions disclosed herein, wherein the form is a modified-release dosage form, wherein the modified-release dosage form is a delayed-release dosage form, an extended-release (ER) dosage form, or a targeted-release dosage form. In some embodiments of the pharmaceutical compositions disclosed herein, wherein the form is an extended-release (ER) dosage form, the ER dosage form is a sustained-release (SR) dosage form or controlled-release (CR) dosage form.

In some embodiments of the pharmaceutical compositions disclosed herein, the form is an I.V. (i.e., infusion) dosage form. In some embodiments of the pharmaceutical composition, wherein the form is an I.V. dosage form, the pH is from about 3.5 to about 14. In some embodiments of the pharmaceutical compositions disclosed herein, the form is an I.V. (i.e., infusion) dosage form. In some embodiments of the pharmaceutical composition, wherein the form is an I.V. dosage form, the pH is from about 5.0 to about 12. In some embodiments of the pharmaceutical compositions disclosed herein, the form is an I.V. (i.e., infusion) dosage form. In some embodiments of the pharmaceutical composition, wherein the form is an I.V. dosage form, the pH is from about 5.5 to about 11. In some embodiments of the pharmaceutical composition, wherein the form is an I.V. dosage form, the pH is about 3.5. In some embodiments of the pharmaceutical composition, wherein the form is an I.V. dosage form, the pH is about 4.0. In some embodiments of the pharmaceutical composition, wherein the form is an I.V. dosage form, the pH is about 4.5. In some embodiments of the pharmaceutical composition, wherein the form is an I.V. dosage form, the pH is about 5.0. In some embodiments of the pharmaceutical composition, wherein the form is an I.V. dosage form, the pH is about 5.5. In some embodiments of the pharmaceutical composition, wherein the form is an I.V. dosage form, the pH is about 6.0. In some embodiments of the pharmaceutical composition, wherein the form is an I.V. dosage form, the pH is about 6.5. In some embodiments of the pharmaceutical composition, wherein the form is an I.V. dosage form, the pH is about 7.0. In some embodiments of the pharmaceutical composition, wherein the form is an I.V. dosage form, the pH is about 7.5. In some embodiments of the pharmaceutical composition, wherein the form is an I.V. dosage form, the pH is about 8.0. In some embodiments of the pharmaceutical composition, wherein the form is an I.V. dosage form, the pH is about 8.5. In some embodiments of the pharmaceutical composition, wherein the form is an I.V. dosage form, the pH is about 9.0. In some embodiments of the pharmaceutical composition, wherein the form is an I.V. dosage form, the pH is about 9.5. In some embodiments of the pharmaceutical composition, wherein the form is an I.V. dosage form, the pH is about 10.0. In some embodiments of the pharmaceutical composition, wherein the form is an I.V. dosage form, the pH is about 10.5. In some embodiments of the pharmaceutical composition, wherein the form is an I.V. dosage form, the pH is about 11.0. In some embodiments of the pharmaceutical composition, wherein the form is an I.V. dosage form, the pH is about 11.5. In some embodiments of the pharmaceutical composition, wherein the form is an I.V. dosage form, the pH is about 12.0. In some embodiments of the pharmaceutical composition, wherein the form is an I.V. dosage form, the pH is about 12.5. In some embodiments of the pharmaceutical composition, wherein the form is an I.V. dosage form, the pH is about 13.0. In some embodiments of the pharmaceutical composition, wherein the form is an I.V. dosage form, the pH is about 13.5. In some embodiments of the pharmaceutical composition, wherein the form is an I.V. dosage form, the pH is about 14.0.

In some embodiments of the pharmaceutical compositions disclosed herein, the pharmaceutical compositions are formulated for systemic delivery. In some embodiments of the pharmaceutical compositions disclosed herein, the pharmaceutical compositions are not formulated for local delivery. In some embodiments of the pharmaceutical compositions disclosed herein, the pharmaceutical compositions, when injected, result in a high level of systemic delivery or exposure without significant depot in the area of injection (i.e., do not provide a high local concentration of a compound of Formula (I), or an isotopic variant thereof; or a metabolite, pharmaceutically acceptable salt, solvate, or hydrate thereof, and/or a TrpV1 modulator (e.g., TrpV1 inhibitor, TrpV1 antagonist, a TrpV1 competitive agonist.) In some embodiments of the pharmaceutical compositions disclosed herein, the pharmaceutical compositions, when injected, result in a high level of systemic delivery or exposure and are not significantly retained in the skin.

In some embodiments of the pharmaceutical compositions disclosed herein, wherein the form is a suspension or a solution, the dosage form comprises a co-solvent.

In some embodiments of the pharmaceutical compositions disclosed herein, wherein the dosage form comprises a co-solvent, the co-solvent comprises PEG200, PEG300, PEG400, PEG600, propylene glycol, ethanol, polysorbate 20, polysorbate 80, cremephor, glycerin, benzyl alcohol, dimethylacetamide (DMA), N-methyl-2-py tTol idone (NMP), tert-butanol, or combinations thereof.

In some embodiments of the pharmaceutical compositions disclosed herein, the dosage form further comprises an oil.

In some embodiments of the pharmaceutical compositions disclosed herein, wherein the dosage form further comprises an oil, the oil comprises sesame oil, soybean oil, vegetable oil, poppyseed oil, safflower oil, or combinations thereof.

The compound of Formula (I) or a hydrate, solvate, tautomer, or pharmaceutically acceptable salt thereof of the present disclosure may be in the form of compositions suitable for administration to a subject. In general, such compositions are “pharmaceutical compositions” comprising a compound of Formula (I) or a hydrate, solvate, tautomer, or pharmaceutically acceptable salt thereof and one or more pharmaceutically acceptable or physiologically acceptable diluents, carriers or excipients. In certain embodiments, the compound of Formula (I) or a hydrate, solvate, tautomer, or pharmaceutically acceptable salt thereof are present in a therapeutically acceptable amount. The pharmaceutical compositions may be used in the methods of the present disclosure; thus, for example, the pharmaceutical compositions can be administered ex vivo or in vivo to a subject in order to practice the therapeutic and prophylactic methods and uses described herein.

The pharmaceutical compositions of the present disclosure can be formulated to be compatible with the intended method or route of administration; exemplary routes of administration are set forth herein.

The pharmaceutical compositions containing the compound of Formula (I) or a hydrate, solvate, tautomer, or pharmaceutically acceptable salt thereof may be in a form suitable for oral use, for example, as tablets, capsules, troches, lozenges, aqueous or oily suspensions, dispersible powders or granules, emulsions, hard or soft capsules, or syrups, solutions, microbeads or elixirs. Pharmaceutical compositions intended for oral use may be prepared according to any method known to the art for the manufacture of pharmaceutical compositions, and such compositions may contain one or more agents such as, for example, sweetening agents, flavoring agents, coloring agents and preserving agents in order to provide pharmaceutically elegant and palatable preparations. Tablets, capsules and the like contain the active ingredient in admixture with non-toxic pharmaceutically acceptable excipients which are suitable for the manufacture thereof. These excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

The tablets, capsules and the like suitable for oral administration may be uncoated or coated by known techniques to delay disintegration and absorption in the gastrointestinal tract and thereby provide a sustained action. For example, a time-delay material such as glyceryl monostearate or glyceryl distearate may be employed. They may also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylene-vinylacetate, methylcellulose, carboxymethylcellulose, protamine sulfate, or lactide/glycolide copolymers, polylactide/glycolide copolymers, or ethylenevinylacetate copolymers in order to control delivery of an administered composition. For example, the oral agent can be entrapped in microcapsules prepared by coacervation techniques or by interfacial polymerization, by the use of hydroxymethylcellulose or gelatin-microcapsules or poly(methylmethacrolate) microcapsules, respectively, or in a colloid drug delivery system. Colloidal dispersion systems include macromolecule complexes, nano-capsules, microspheres, microbeads, and lipid-based systems, including oil-in-water emulsions, micelles, mixed micelles, and liposomes. Methods for the preparation of the above-mentioned formulations will be apparent to those skilled in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture thereof. Such excipients can be suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents, such as those set forth above, and flavoring agents may be added to provide a palatable oral preparation.

Dispersible powders and granules suitable for preparation of an aqueous suspension by the addition of water provide the active ingredient in admixture with a dispersing or wetting agent, and optionally one or more suspending agents and/or preservatives. Suitable dispersing or wetting agents and suspending agents are exemplified herein.

The pharmaceutical compositions of the present disclosure may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

The pharmaceutical compositions typically comprise a therapeutically effective amount of a compound of Formula (I) or a hydrate, solvate, tautomer, or pharmaceutically acceptable salt thereof, and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate-buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer; N-(2-Hydroxyethyl)piperazine-N′-(2-ethanesulfonic acid) (HEPES); 2-(N-Morpholino)ethanesulfonic acid (MES); 2-(N-Morpholino)ethanesulfonic acid sodium salt (MES); 3-(N-Morpholino)propanesulfonic acid (MOPS); and N-tris[Hydroxymethyl]methyl-3-aminopropanesulfonic acid (TAPS).

After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampule, syringe, or autoinjector (similar to, e.g., an EpiPen®)), whereas a multi-use container (e.g., a multi-use vial) is provided in other embodiments.

Formulations can also include carriers to protect the composition against rapid degradation or elimination from the body, such as a controlled release formulation, including liposomes, hydrogels, prodrugs and microencapsulated delivery systems. For example, a time-delay material such as glyceryl monostearate or glyceryl stearate alone, or in combination with a wax, may be employed. Any drug delivery apparatus may be used to deliver a compound of Formula (I) or a hydrate, solvate, tautomer, or pharmaceutically acceptable salt thereof, including implants (e.g., implantable pumps) and catheter systems, slow injection pumps and devices, all of which are well known to the skilled artisan.

Depot injections, which are generally administered subcutaneously or intramuscularly, may also be utilized to release the compound (e.g., a compound of Formula (I) or a hydrate, solvate, tautomer, or pharmaceutically acceptable salt thereof) disclosed herein over a defined period of time. Depot injections are usually either solid- or oil-based and generally comprise at least one of the formulation components set forth herein. One of ordinary skill in the art is familiar with possible formulations and uses of depot injections.

The pharmaceutical compositions may be in the form of a sterile injectable aqueous or oleagenous suspension. This suspension may be formulated according to the known art using those suitable dispersing or wetting agents and suspending agents mentioned herein. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, Cremophor® EL (BASF, Parsippany, N.J.) or phosphate buffered saline (PBS), ethanol, polyol (e.g., glycerol, propylene glycol, and liquid polyethylene glycol), and suitable mixtures thereof. In addition, sterile fixed oils are conventionally employed as a solvent or suspending medium; for this purpose, any bland fixed oil may be employed, including synthetic mono- or diglycerides. Moreover, fatty acids, such as oleic acid, find use in the preparation of injectables. Prolonged absorption of particular injectable formulations can be achieved by including an agent that delays absorption (e.g., aluminum monostearate or gelatin).

The present disclosure contemplates the administration of the compound of Formula (I) or a hydrate, solvate, tautomer, or pharmaceutically acceptable salt thereof in the form of suppositories for rectal administration. The suppositories can be prepared by mixing the drug with a suitable non-irritating excipient, which is solid at ordinary temperatures but liquid at the rectal temperature and will therefore melt in the rectum to release the drug. Such materials include, but are not limited to, cocoa butter and polyethylene glycols.

The compound of Formula (I) or a hydrate, solvate, tautomer, or pharmaceutically acceptable salt thereof may be in the form of any other suitable pharmaceutical composition (e.g., sprays for nasal or inhalation use) currently known or developed in the future

### Methods of Use

In an aspect, provided herein is a method of inhibiting mitochondrial complex I, comprising contacting the mitochondrial complex I with a compound of structural Formula (I):

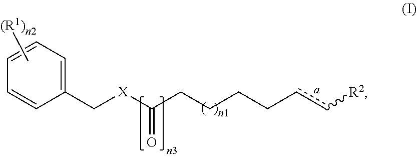

or an isotopic variant thereof; or a metabolite, pharmaceutically acceptable salt, solvate, or hydrate thereof,

wherein:
[symbol] is a single bond or double bond;

n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl.

In some embodiments, the compound of structural Formula I has the structure:

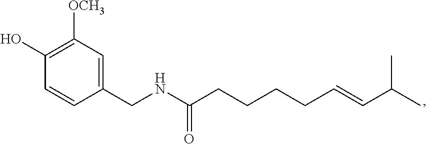 [SMILES: COc1cc(CNC(=O)CCCC/C=C/C(C)C)ccc1O]

or an isotopic variant thereof; or a metabolite, pharmaceutically acceptable salt, solvate, or hydrate thereof.

In some embodiments, at least 10 μM of a compound of structural Formula (I), or an isotopic variant thereof; or a metabolite, pharmaceutically acceptable salt, solvate, or hydrate thereof, is used in the method of inhibiting mitochondrial complex I. In some embodiments, at least 15 μM of a compound of structural Formula (I), or an isotopic variant thereof; or a metabolite, pharmaceutically acceptable salt, solvate, or hydrate thereof, is used in the method of inhibiting mitochondrial complex I. In some embodiments, at least 20 μM of a compound of structural Formula (I), or an isotopic variant thereof; or a metabolite, pharmaceutically acceptable salt, solvate, or hydrate thereof, is used in the method of inhibiting mitochondrial complex I. In some embodiments, at least 25 μM of a compound of structural Formula (I), or an isotopic variant thereof; or a metabolite, pharmaceutically acceptable salt, solvate, or hydrate thereof, is used in the method of inhibiting mitochondrial complex I. In some embodiments, at least 30 μM of a compound of structural Formula (I), or an isotopic variant thereof; or a metabolite, pharmaceutically acceptable salt, solvate, or hydrate thereof, is used in the method of inhibiting mitochondrial complex I. In some embodiments, at least 35 μM of a compound of structural Formula (I), or an isotopic variant thereof; or a metabolite, pharmaceutically acceptable salt, solvate, or hydrate thereof, is used in the method of inhibiting mitochondrial complex I. In some embodiments, at least 40 μM of a compound of structural Formula (I), or an isotopic variant thereof; or a metabolite, pharmaceutically acceptable salt, solvate, or hydrate thereof, is used in the method of inhibiting mitochondrial complex I. In some embodiments, at least 45 μM of a compound of structural Formula (I), or an isotopic variant thereof; or a metabolite, pharmaceutically acceptable salt, solvate, or hydrate thereof, is used in the method of inhibiting mitochondrial complex I. In some embodiments, at least 45 μM of a compound of structural Formula (I), or an isotopic variant thereof; or a metabolite, pharmaceutically acceptable salt, solvate, or hydrate thereof, is used in the method of inhibiting mitochondrial complex I. In some embodiments, at least 50 ᵢtM of a compound of structural Formula (I), or an isotopic variant thereof; or a metabolite, pharmaceutically acceptable salt, solvate, or hydrate thereof, is used in the method of inhibiting mitochondrial complex I.

In an aspect, provided herein is a method of a method of treating and/or preventing a capsaicin-responsive disease or disorder, comprising administering to a subject in need thereof a pharmaceutical composition or a supplement (e.g., a nutritional supplement) as disclosed herein.

In some embodiments, the disease or disorder is a cell proliferative disease, obesity, diabetes, a bacterial infection, a cardiovascular disease, a neurodegenerative disease or disorder, an inflammatory disease or disorder, a comorbidity, an autoimmune disease, hypercholesterolemia or a mood disorder. In some embodiments, the disease or disorder is an infectious disease or disorder (e.g., influenza or coronavirus). In some embodiments, the disease or disorder is a coagulant disease or disorder.

In some embodiments, the mood disorder is depression. In some embodiments, the inflammatory disease or disorder is rheumatoid arthritis. In some embodiments, the cell proliferative disease is a cancer. In some embodiments, the disorder is diabetes or obesity. In some embodiments, the comorbidity is chronic kidney disease, stroke, congestive heart failure, dementia, schizophrenia, hepatitis, autism spectrum disorder, HIV, or a combination thereof.

In some embodiments, the supplements disclosed herein are for use in the treatment of a capsaicin-responsive disease or disorder.

In some embodiments, the supplements disclosed herein are for use in the treatment of obesity, diabetes, a bacterial infection, a cardiovascular disease, a neurodegenerative disease or disorder, an inflammatory disease or disorder, a comorbidity, an autoimmune disease, hypercholesterolemia or a mood disorder. In some embodiments, the supplements disclosed herein are used as an anti-coagulant.

In some embodiments, the supplements disclosed herein are used for supporting the cognitive health of a human In some embodiments, the supplements disclosed herein are used for supporting clarity and/or concentration of a human In some embodiments, the supplements disclosed herein are used for promoting the longevity of a human In some embodiments, the supplements disclosed herein are used for boosting alertness or wakefulness of a human. In some embodiments, the supplements disclosed herein are used for supporting a healthy weight in a human. In some embodiments, the supplements disclosed herein are used for promoting the microbiome health of a human. In some embodiments, the supplements disclosed herein are used muscle energetic enhancers of a human.

In some embodiments, the mood disorder is depression. In some embodiments, the inflammatory disease or disorder is rheumatoid arthritis. In some embodiments, the cell proliferative disease is a cancer. In some embodiments, the disorder is diabetes or obesity.

In an aspect, provided herein is a method of treating or preventing a capsaicin-responsive disease or disorder, comprising administering to a subject in need thereof:
(i) a compound of structural Formula (I):

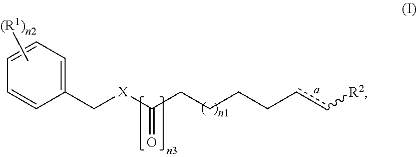

or an isotopic variant thereof; or a metabolite, pharmaceutically acceptable salt, solvate, or hydrate thereof,
   wherein:
[symbol] is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl; and
(ii) at least one additional therapeutic agent selected from:
   a transient receptor potential cation channel subfamily V member 1 (TrpV1) modulator,
   a fatty acid amide hydrolase (FAAH) receptor inhibitor; and
   a specialized pro-resolving mediator (SPM), or a combination thereof,
      wherein when the TrpV1 modulator is a fatty acid, then the compound of structural Formula (I) or an isotopic variant thereof; or a metabolite, pharmaceutically acceptable salt, solvate, or hydrate thereof, and the TrpV1 modulator are each individually formulated.

In some embodiments, R^{1.1} is CH₃; and R^{1.2} is OH.

In some embodiments, X is O. In some embodiments, X is NH. In some embodiments, [symbol] is a double bond. In some embodiments, [symbol] is a single bond. In some embodiments, n1 is 2. In some embodiments, n1 is 1. In some embodiments, n1 is 0. In some embodiments, R² is unsubstituted alkyl. In some embodiments, R² is —CH(CH₃)₂ or —CH₃.

In some embodiments, the compound is at least one of:

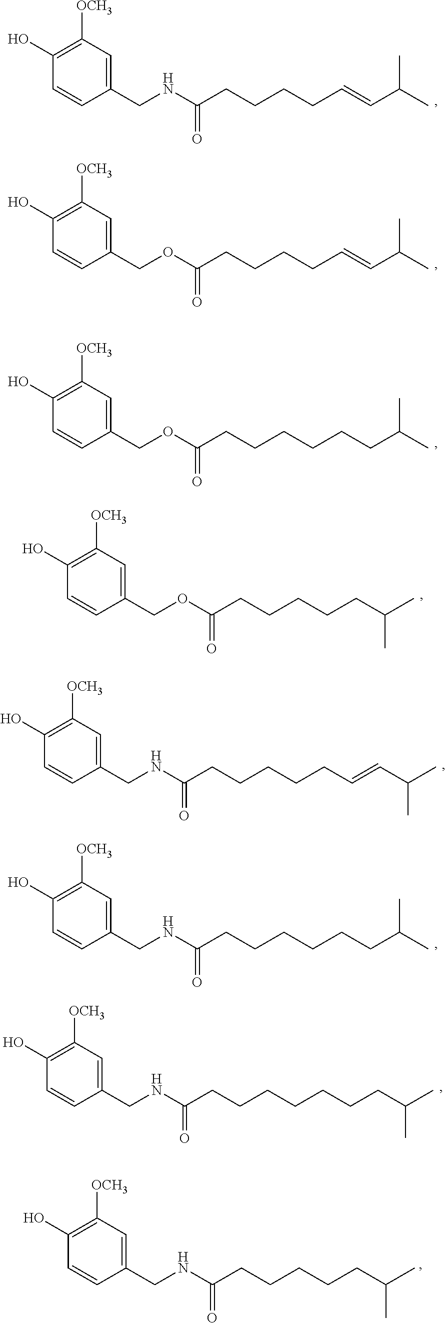

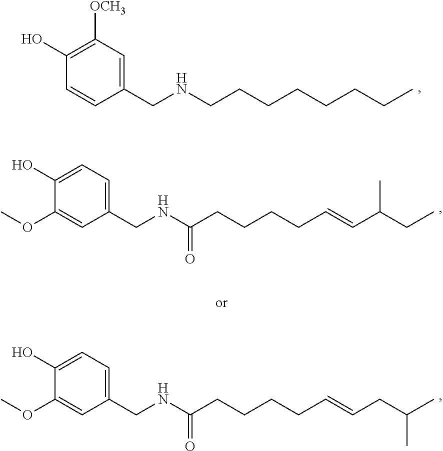

or an isotopic variant thereof; or a metabolite, pharmaceutically acceptable salt, solvate, or hydrate thereof.

In some embodiments, the compound is:

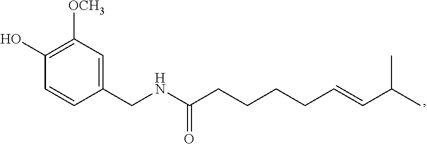 [SMILES: COc1cc(CNC(=O)CCCC/C=C/C(C)C)ccc1O]

or an isotopic variant thereof; or a metabolite, pharmaceutically acceptable salt, solvate, or hydrate thereof.

In some embodiments, the TrpV1 modulator is derived from a foodstuff, foodstuff extract, a fruit extract, a vegetable extract, and/or a plant extract including but not limited to almonds, avocado, and/or olive oil.

In an aspect, provided herein is a method of treating a capsaicin-responsive disease or disorder, comprising subjecting a patient in need thereof to cryotherapy and administering a compound of Formula (I), or an isotopic variant thereof; or a metabolite, pharmaceutically acceptable salt, solvate, or hydrate thereof.

In an aspect, provided herein is a method of treating a capsaicin-responsive disease or disorder, comprising to a subject in need thereof a compound of Formula (I), or an isotopic variant thereof; or a metabolite, pharmaceutically acceptable salt, solvate, or hydrate thereof, and milk, or a milk-derived product including but not limited to casein. In an aspect, provided herein is a method of treating a capsaicin-responsive disease or disorder, comprising to a subject in need thereof a compound of Formula (I), or an isotopic variant thereof; or a metabolite, pharmaceutically acceptable salt, solvate, or hydrate thereof, and peanuts, or a peanut-derived product including but not limited to peanut butter.

In some embodiments, the at least one additional therapeutic agent is a TrpV1 modulator.

In some embodiments, the TrpV1 modulator is a TrpV1 antagonist, a TrpV1 competitive agonist, or combination thereof.

In some embodiments, the TrpV1 modulator is a fatty acid triglyceride, or spilanthol.

In some embodiments, the fatty acid is a polyunsaturated fatty acid (PUFA).

In some embodiments, the PUFA is anandamide.

In some embodiments, the fatty acid is an omega-9 fatty acid.

In some embodiments, the fatty acid is oleic acid or erucic acid.

In some embodiments, the at least one additional therapeutic agent is a FAAH receptor inhibitor.

In some embodiments, the FAAH receptor inhibitor is SSR411298.

In some embodiments, the at least one additional therapeutic agent is a FAAH inhibitor.

In some embodiments, the FAAH inhibitor is a flavonoid. Exemplary flavonoids include, but are not limited to, myricetin, quercetin, kaempferol, luteolin, and apigenin. In some embodiments, the FAAH inhibitor is kaempferol.

In some embodiments, the at least one additional therapeutic agent is an SPM.

In some embodiments, the SPM is maresin.

In some embodiments, the compound of structural Formula (I) or an isotopic variant thereof; or a metabolite, pharmaceutically acceptable salt, solvate, or hydrate thereof, is administered orally.

In some embodiments, the compound of structural Formula (I) or an isotopic variant thereof; or a metabolite, pharmaceutically acceptable salt, solvate, or hydrate thereof, is administered orally via a suspension or solution.

In some embodiments, the compound of structural Formula (I) or an isotopic variant thereof; or a metabolite, pharmaceutically acceptable salt, solvate, or hydrate thereof, is administered orally via a solid dosage form. In some embodiments, the solid dosage form is a tablet or capsule.

In some embodiments, the compound of structural Formula (I) or an isotopic variant thereof; or a metabolite, pharmaceutically acceptable salt, solvate, or hydrate thereof, is administered by inhalation, nebulization, or nasal delivery.

In some embodiments, the compound of structural Formula (I) or an isotopic variant thereof; or a metabolite, pharmaceutically acceptable salt, solvate, or hydrate thereof is provided as an aerosol. In some embodiments, the compound of structural Formula (I) or an isotopic variant thereof; or a metabolite, pharmaceutically acceptable salt, solvate, or hydrate thereof is provided as an inhalable aerosol. In some embodiments, the compound of structural Formula (I) or an isotopic variant thereof; or a metabolite, pharmaceutically acceptable salt, solvate, or hydrate thereof is in a form of an electronic delivery system. In some embodiments, the compound of structural Formula (I) or an isotopic variant thereof; or a metabolite, pharmaceutically acceptable salt, solvate, or hydrate thereof is administered using a vaping device. In some embodiments, the compound of structural Formula (I) or an isotopic variant thereof; or a metabolite, pharmaceutically acceptable salt, solvate, or hydrate thereof, is administered using an e-cigarette.

In some embodiments, the compound of structural Formula (I) or an isotopic variant thereof; or a metabolite, pharmaceutically acceptable salt, solvate, or hydrate thereof, is administered by inhalation via a vaporizer.

In some embodiments, the compound of structural Formula (I) or an isotopic variant thereof; or a metabolite, pharmaceutically acceptable salt, solvate, or hydrate thereof, is administered parenterally.

In some embodiments, the compound of structural Formula (I) or an isotopic variant thereof; or a metabolite, pharmaceutically acceptable salt, solvate, or hydrate thereof, is administered via parenteral injection, infusion, or implantation.

In some embodiments, the parenteral injection is intravenous, intramuscular, subcutaneous, or intradermal.

In some embodiments, the compound of structural Formula (I) or an isotopic variant thereof; or a metabolite, pharmaceutically acceptable salt, solvate, or hydrate thereof, is administered via a suppository.

In some embodiments, the compound of structural Formula (I) or an isotopic variant thereof; or a metabolite, pharmaceutically acceptable salt, solvate, or hydrate thereof, is administered cutaneous or transdermal delivery.

In some embodiments, the compound of structural Formula (I) or an isotopic variant thereof; or a metabolite, pharmaceutically acceptable salt, solvate, or hydrate thereof, is administered by sublingual or buccal delivery.

In some embodiments, the compound of Formula (I), or an isotopic variant thereof; or a metabolite, pharmaceutically acceptable salt, solvate, or hydrate thereof is present in the composition in an amount of greater than about 10% (w/v).

In some embodiments, the compound of Formula (I), or an isotopic variant thereof; or a metabolite, pharmaceutically acceptable salt, solvate, or hydrate thereof is present in the composition in an amount of greater than about 20% (w/v).

In some embodiments, the compound of Formula (I), or an isotopic variant thereof; or a metabolite, pharmaceutically acceptable salt, solvate, or hydrate thereof is present in the composition in an amount of greater than about 30% (w/v).

In some embodiments, the compound of Formula (I), or an isotopic variant thereof; or a metabolite, pharmaceutically acceptable salt, solvate, or hydrate thereof is present in the composition in an amount of greater than about 40% (w/v).

In some embodiments, the compound of Formula (I), or an isotopic variant thereof; or a metabolite, pharmaceutically acceptable salt, solvate, or hydrate thereof is present in the composition in an amount of from about 10% (w/v) to about 50% (w/v). In some embodiments, the compound of Formula (I), or an isotopic variant thereof; or a metabolite, pharmaceutically acceptable salt, solvate, or hydrate thereof is present in the composition in an amount of from about 20% (w/v) to about 50% (w/v). In some embodiments, the compound of Formula (I), or an isotopic variant thereof; or a metabolite, pharmaceutically acceptable salt, solvate, or hydrate thereof is present in the composition in an amount of from about 30% (w/v) to about 50% (w/v). In some embodiments, the compound of Formula (I), or an isotopic variant thereof; or a metabolite, pharmaceutically acceptable salt, solvate, or hydrate thereof is present in the composition in an amount of from about 40% (w/v) to about 50% (w/v).

In some embodiments, the ratio of the therapeutic agent to a compound of Formula (I), or an isotopic variant thereof; or a metabolite, pharmaceutically acceptable salt, solvate, or hydrate thereof, is about 1:1. In some embodiments, the ratio of the therapeutic agent to a compound of Formula (I), or an isotopic variant thereof; or a metabolite, pharmaceutically acceptable salt, solvate, or hydrate thereof, is about 1.1:1. In some embodiments, the ratio of the therapeutic agent to a compound of Formula (I), or an isotopic variant thereof; or a metabolite, pharmaceutically acceptable salt, solvate, or hydrate thereof, is about 1.2:1. In some embodiments, the ratio of the therapeutic agent to a compound of Formula (I), or an isotopic variant thereof; or a metabolite, pharmaceutically acceptable salt, solvate, or hydrate thereof, is about 1.3:1. In some embodiments, the ratio of the therapeutic agent to a compound of Formula (I), or an isotopic variant thereof; or a metabolite, pharmaceutically acceptable salt, solvate, or hydrate thereof, is about 1.4:1. In some embodiments, the ratio of the therapeutic agent to a compound of Formula (I), or an isotopic variant thereof; or a metabolite, pharmaceutically acceptable salt, solvate, or hydrate thereof, is about 1.5:1.

In some embodiments, the disease or disorder is a cell proliferative disease, obesity, diabetes, a bacterial infection, a cardiovascular disease, a neurodegenerative disease or disorder, an inflammatory disease or disorder, a comorbidity, an autoimmune disease, hypercholesterolemia or a mood disorder. In some embodiments, the mood disorder is depression. In some embodiments, the inflammatory disease or disorder is rheumatoid arthritis. In some embodiments, the cell proliferative disease is a cancer. In some embodiments, the disorder is diabetes or obesity.

In some embodiments, the compound of Formula (I), or a pharmaceutically acceptable salt thereof, and the at least one additional therapeutic agent are administered simultaneously, approximately simultaneously, or sequentially, in any order.

In some embodiments, the compound of Formula (I), or a pharmaceutically acceptable salt thereof, and the at least one additional therapeutic agent are administered simultaneously or approximately simultaneously.

In some embodiments, the compound of Formula (I), or a pharmaceutically acceptable salt thereof, and the at least one additional therapeutic agent are administered sequentially.

In some embodiments, the compound of Formula (I), or a pharmaceutically acceptable salt thereof, is released before the at least one additional therapeutic agent.

In some embodiments, the compound of Formula (I), or a pharmaceutically acceptable salt thereof, is released after the at least one additional therapeutic agent

In some embodiments of the methods described herein, the compound of Formula I, or a hydrate, solvate, tautomer, or pharmaceutically acceptable salt thereof, is administered to the subject daily.

In some embodiments of the methods described herein, the compound of Formula I, or a hydrate, solvate, tautomer, or pharmaceutically acceptable salt thereof, is administered to the subject once per day, twice per day, three times per day, or four times per day.

In some embodiments of the methods described herein, the compound of Formula I, or a hydrate, solvate, tautomer, or pharmaceutically acceptable salt thereof, is administered to the subject for a period of at least one week.

In some embodiments of the methods described herein, the compound of Formula I, or a hydrate, solvate, tautomer, or pharmaceutically acceptable salt thereof, is administered to the subject for a period of one week, two weeks, 6 weeks, 12 weeks, 24 weeks, 48 weeks, or 52 weeks. Dosing

In some embodiments, the compound of Formula (I) or a hydrate, solvate, tautomer, or pharmaceutically acceptable salt thereof pharmaceutical compositions described herein are provided at the maximum tolerated dose (MTD) for the compound of Formula (I). In other embodiments, the amount of the compound of Formula (I) or a hydrate, solvate, tautomer, or pharmaceutically acceptable salt thereof pharmaceutical composition administered is from about 10% to about 90% of the maximum tolerated dose (MTD), from about 25% to about 75% of the MTD, or about 50% of the MTD. In some other embodiments, the amount of the compound of Formula (I) or a hydrate, solvate, tautomer, or pharmaceutically acceptable salt thereof pharmaceutical compositions administered is from about 5%, 10%, 15%, 20%, 25%, 30%, 35%, 40%, 45%, 50%, 55%, 60%, 65%, 70%, 75%, 80%, 85%, 90%, 95%, 99%, or higher, or any range derivable therein, of the MTD for the compound of Formula (I). Percentage can be weight by weight or weight by volume.

In certain embodiments, in the treatment, prevention, or amelioration of one or more symptoms of the disorders, diseases, or conditions described herein, an appropriate dosage level of the compound of Formula (I) or a hydrate, solvate, tautomer, or pharmaceutically acceptable salt thereof generally is ranging from about 1 to 8000 mg, from about 1 to about 1000 mg, from about 5 to about 1000 mg, from about 5 to about 800 mg, from about 5 to about 600 mg, from about 5 to about 500 mg or from about 50 to about 500 mg which can be administered in single or multiple doses. In certain embodiments, the compound of Formula (I) or a hydrate, solvate, tautomer, or pharmaceutically acceptable salt thereof is administered in an amount of about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 240, about 250, about 260, about 270, about 275, about 280, about 290, about 300, about 310, about 320, about 330, about 340, about 350, about 360 mg, about 370 mg, about 380 mg, about 390 mg, about 400 mg, 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, 910 mg, about 920 mg, about 930 mg, about 940 mg, about 950 mg, about 960 mg, about 970 mg, about 980 mg, about 990 mg, about 1000, about 1200, about 1400, about 1500, about 1600, about 1700, about 1800, about 1900, about 2000, about 2100, about 2200, about 2400, about 2500, about 2600, about 2800, about 3000, about 4000, about 5000, about 6000, about 7000, about 8000 mg, or any range derivable therein.

In certain embodiments, the compound of Formula (I) or a hydrate, solvate, tautomer, or pharmaceutically acceptable salt thereof is administered in an amount of about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 240 mg, about 250 mg, about 260 mg, about 270 mg, about 275 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, 910 mg, about 920 mg, about 930 mg, about 940 mg, about 950 mg, about 960 mg, about 970 mg, about 980 mg, about 990 mg, about 1000, about 1200, about 1400, about 1500, about 1600, about 1700, about 1800, about 1900, about 2000, about 2100, about 2200, about 2400, about 2500, about 2600, about 2800, about 3000, about 4000, about 5000, about 6000, about 7000, about 8000 mg mg/day.

For oral administration, the pharmaceutical compositions provided herein can be formulated in the form of tablets or capsules containing from about 1.0 to about 1,000 mg of a compound of Formula (I) or a hydrate, solvate, tautomer, or pharmaceutically acceptable salt thereof, in one embodiment, 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, 910 mg, about 920 mg, about 930 mg, about 940 mg, about 950 mg, about 960 mg, about 970 mg, about 980 mg, about 990 mg, about 1000 mg, or about 2000 mg of the compound of Formula (I) or a hydrate, solvate, tautomer, or pharmaceutically acceptable salt thereof for the symptomatic adjustment of the dosage to the patient to be treated.

In some embodiments, the compound of Formula (I) or a hydrate, solvate, tautomer, or pharmaceutically acceptable salt thereof is administered in an amount of about or at most 1 mg/day, about 5 mg/day, about 10 mg/day, about 15 mg/day, about 20 mg/day, about 25 mg/day, about 30 mg/day, about 35 mg/day, about 40 mg/day, about 45 mg/day, about 50 mg/day, about 55 mg/day, about 60 mg/day, about 65 mg/day, about 70 mg/day, about 75 mg/day, about 80 mg/day, about 85 mg/day, about 90 mg/day, about 95 mg/day, about 100 mg/day, about 105 mg/day, about 110 mg/day, about 115 mg/day, about 120 mg/day, about 125 mg/day, about 130 mg/day, about 135 mg/day, about 140 mg/day, about 145 mg/day, about 150 mg/day, about 155 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, 410 mg/day, about 420 mg/day, about 430 mg/day, about 440 mg/day, about 450 mg/day, about 460 mg/day, about 470 mg/day, about 480 mg/day, about 490 mg/day, about 500 mg/day, about 510 mg/day, about 520 mg/day, about 530 mg/day, about 540 mg/day, about 550 mg/day, about 560 mg/day, about 570 mg/day, about 580 mg/day, about 590 mg/day, or about 600 mg/day. In some embodiments, the compound of Formula (I) is administered in a range of about 1 mg/day to about 20 mg/day, about 1 mg/day to about 50 mg/day about 1 mg/day to about 100 mg/day, about 10 mg/day to about 100 mg/day, about 50 mg/day to about 500 mg/day, about 50 mg/day to about 250 mg/day, about 100 mg/day to about 500 mg/day or about 100 mg/day to about 200 mg/day.

In some embodiments, the compound of Formula (I) or a hydrate, solvate, tautomer, or pharmaceutically acceptable salt thereof is derived from a natural source such that about or at most 1 mg/day, about 5 mg/day, about 10 mg/day, about 15 mg/day, about 20 mg/day, about 25 mg/day, about 30 mg/day, about 35 mg/day, about 40 mg/day, about 45 mg/day, about 50 mg/day, about 55 mg/day, about 60 mg/day, about 65 mg/day, about 70 mg/day, about 75 mg/day, about 80 mg/day, about 85 mg/day, about 90 mg/day, about 95 mg/day, about 100 mg/day, about 105 mg/day, about 110 mg/day, about 115 mg/day, about 120 mg/day, about 125 mg/day, about 130 mg/day, about 135 mg/day, about 140 mg/day, about 145 mg/day, about 150 mg/day, about 155 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, 410 mg/day, about 420 mg/day, about 430 mg/day, about 440 mg/day, about 450 mg/day, about 460 mg/day, about 470 mg/day, about 480 mg/day, about 490 mg/day, about 500 mg/day, about 510 mg/day, about 520 mg/day, about 530 mg/day, about 540 mg/day, about 550 mg/day, about 560 mg/day, about 570 mg/day, about 580 mg/day, about 590 mg/day, or about 600 mg/day Formula (I) or a hydrate, solvate, tautomer, or pharmaceutically acceptable salt thereof is administered. For example, Formula (I) or a hydrate, solvate, tautomer, or pharmaceutically acceptable salt thereof is derived from chili, cayenne pepper, red pepper, oleoresin red pepper, tomato, or combinations thereof. In some embodiments, Formula (I) or a hydrate, solvate, tautomer, or pharmaceutically acceptable salt thereof is derived from Capsicum Frutescens or Oleoresin Capsicum.

In some embodiments, the natural source comprises at least or about 1 mg/g, about 5 mg/g, about 10 mg/g, about 15 mg/g, about 20 mg/g, about 25 mg/g, about 30 mg/g, about 35 mg/g, about 40 mg/g, about 45 mg/g, about 50 mg/g, about 55 mg/g, about 60 mg/g, about 65 mg/g, about 70 mg/g, about 75 mg/g, or about 80 mg/g of a compound of Formula (I) or a hydrate, solvate, tautomer, or pharmaceutically acceptable salt thereof. In some embodiments, the natural source comprises at least or about 5%, 10%, 20%, 30%, 40%, 50%, 60%, 70%, 80%, 90%, 95%, or more than 95% of a compound of Formula (I) or a hydrate, solvate, tautomer, or pharmaceutically acceptable salt thereof.

In some embodiments, various heat units of the compound of Formula (I) or a hydrate, solvate, tautomer, or pharmaceutically acceptable salt thereof is administered. In some embodiments, the compound of Formula (I) or a hydrate, solvate, tautomer, or pharmaceutically acceptable salt thereof comprises a Scoville Heat Unit (SHU) of about 0 to about 16 M SHU. In some embodiments, the compound of Formula (I) or a hydrate, solvate, tautomer, or pharmaceutically acceptable salt thereof comprises a Scoville Heat Unit (SHU) of at least or about 1, 2, 3, 4, 5, 6, 7, 8, 9, 10, 12, 14, 16, or more than 16 M SHU. SHU, in some embodiments, provides the amount of sugar water to dilute a volume of pepper extract. For example, 100 SHU indicates that 100 mL of sugar water is needed to dilute 1 mL of pepper extract.

In some embodiments, the pharmaceutical compositions provided herein can be formulated in the form of an injection or I.V. infusion containing a compound of Formula (I) or a hydrate, solvate, tautomer, or pharmaceutically acceptable salt thereof. The dose per day described herein may be given once per day or multiple times per day in the form of sub-doses given b.i.d., t.i.d., q.i.d., or the like where the number of sub-doses equal the dose per day. The pharmaceutical compositions can be administered on a regimen of 1 to 4 times per day, including once, twice, three times, and four times per day. In some embodiments, the pharmaceutical compositions are administered once per day. In some embodiments, the pharmaceutical compositions are administered twice per day. In some embodiments, the pharmaceutical compositions are administered three times per day. In some embodiments, a loading dose, followed by maintenance doses, of the pharmaceutical compositions are administered.

In some embodiments, the pharmaceutical compositions provided herein are administered as a tablet. In some embodiments, the pharmaceutical compositions provided herein are administered as a capsule. In some embodiments, the pharmaceutical compositions provided herein are administered as an injection. In some embodiments, the pharmaceutical compositions provided herein are administered as an I.V. infusion.

In some embodiments, the pharmaceutical compositions provided herein are administered as an I.V. infusion over a period of about 15 minutes. In some embodiments, the pharmaceutical compositions provided herein are administered as an I.V. infusion over a period of about 30 minutes. In some embodiments, the pharmaceutical compositions provided herein are administered as an I.V. infusion over a period of about 45 minutes. In some embodiments, the pharmaceutical compositions provided herein are administered as an I.V. infusion over a period of about one hour. In some embodiments, the pharmaceutical compositions provided herein are administered as an I.V. infusion over a period of about two hours. In some embodiments, the pharmaceutical compositions provided herein are administered as an I.V. infusion over a period of about three hours or more.

In some embodiments, the pharmaceutical compositions provided herein are administered for about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, or about 30 days.

In some embodiments, the pharmaceutical compositions provided herein are administered for administered daily, every other day, every other day 3 times a week, every 2 weeks, every 3 weeks, every 4 weeks, every 5 weeks, every 3 days, every 4 days, every 5 days, every 6 days, weekly, bi-weekly, 3 times a week, 4 times a week, 5 times a week, 6 times a week, once a month, twice a month, 3 times a month, once every 2 months, once every 3 months, once every 4 months, once every 5 months, or once every 6 months.

In some instances, the method for the administration of multiple compounds (e.g., a compound of Formula (I) or an isotopic variant thereof; or a metabolite, pharmaceutically acceptable salt, solvate, or hydrate thereof) comprises administering compounds within 48 hours or less of each other. In some embodiments administration occurs within 24 hours, 12 hours, 6 hours, 3 hours, 1 hour, or 15 minutes. In some instances, the compounds are administered simultaneously. One example of simultaneous administration is the injection of one compound immediately before, after, or during the oral administration of the second compound, immediately referring to a time less than about 5 minutes.

In some embodiments of the pharmaceutical compositions described herein, a second dose is administered about 1 hour after the first dose, a second dose is administered about 2 hours after the first dose, a second dose is administered about 3 hours after the first dose, a second dose is administered about 4 hours after the first dose, a second dose is administered about 5 hours after the first dose, a second dose is administered about 6 hours after the first dose, a second dose is administered about 7 hours after the first dose, a second dose is administered about 8 hours after the first dose, a second dose is administered about 9 hours after the first dose, a second dose is administered about 10 hours after the first dose, a second dose is administered about 11 hours after the first dose, or a second dose is administered about 12 hours after the first dose. In some embodiments of the pharmaceutical compositions described herein, the second dose is administered about 1, about 2, about 3, about 4, about 5, about 6, about 7, about 8, about 9, about 10, about 11, or about 12 hours after completion of administration of the first dose by I.V. infusion. In some embodiments of the pharmaceutical compositions, the second dose is administered about 1, about 2, about 3, about 4, about 5, about 6, about 7, about 8, about 9, about 10, about 11, or about 12 hours after initiation of administration of the first dose by I.V. infusion.

### Subjects

In some embodiments, the subject is immunocompromised. In some embodiments, the subject is an immunocompromised human subject. In some embodiments, the human subject is under the age of 1 year. In some embodiments, the human subject is an infant under 1 month old. In some embodiments, the human subject is over the age of 70 years.

In some embodiments, the subject is a mammal. In some embodiments, the mammal is a human.

It is frequently beneficial to improve one of more physical properties of the treatment modalities disclosed herein and/or the manner in which they are administered. Improvements of physical properties include, for example, methods of increasing water solubility, bioavailability, serum half-life, and/or therapeutic half-life; and/or modulating biological activity. Modifications known in the art include pegylation, Fc-fusion and albumin fusion. Although generally associated with large molecule agents (e.g., polypeptides), such modifications have recently been evaluated with particular small molecules. By way of example, Chiang, M. et al. (J. Am. Chem. Soc., 2014, 136(9):3370-73) describe a small molecule agonist of the adenosine 2a receptor conjugated to the immunoglobulin Fc domain. The small molecule-Fc conjugate retained potent Fc receptor and adenosine 2a receptor interactions and showed superior properties compared to the unconjugated small molecule. Covalent attachment of PEG molecules to small molecule therapeutics has also been described (Li, W. et al., Progress in Polymer Science, 2013 38:421-44).

The compounds of the present disclosure may be administered to a subject in an amount that is dependent upon, for example, the goal of administration (e.g., the degree of resolution desired); the age, weight, sex, and health and physical condition of the subject to which the formulation is being administered; the route of administration; and the nature of the disease, disorder, condition or symptom thereof. The dosing regimen may also take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan.

In general, dosing parameters dictate that the dosage amount be less than an amount that could be irreversibly toxic to the subject (the maximum tolerated dose (MTD)) and not less than an amount required to produce a measurable effect on the subject. Such amounts are determined by, for example, the pharmacokinetic and pharmacodynamic parameters associated with ADME, taking into consideration the route of administration and other factors.

An effective dose (ED) is the dose or amount of an agent that produces a therapeutic response or desired effect in some fraction of the subjects taking it. The “median effective dose” or ED₅₀ of an agent is the dose or amount of an agent that produces a therapeutic response or desired effect in 50% of the population to which it is administered. Although the ED₅₀ is commonly used as a measure of reasonable expectance of an agent's effect, it is not necessarily the dose that a clinician might deem appropriate taking into consideration all relevant factors. Thus, in some situations the effective amount is more than the calculated ED₅₀, in other situations the effective amount is less than the calculated ED₅₀, and in still other situations the effective amount is the same as the calculated ED_{50.}

In addition, an effective dose of the compounds of the present disclosure may be an amount that, when administered in one or more doses to a subject, produces a desired result relative to a healthy subject. For example, for a subject experiencing a particular disorder, an effective dose may be one that improves a diagnostic parameter, measure, marker and the like of that disorder by at least about 5%, at least about 10%, at least about 20%, at least about 25%, at least about 30%, at least about 40%, at least about 50%, at least about 60%, at least about 70%, at least about 80%, at least about 90%, or more than 90%, where 100% is defined as the diagnostic parameter, measure, marker and the like exhibited by a normal subject.

In embodiments, the dosage of the desired compound is contained in a “unit dosage form.” The phrase “unit dosage form” refers to physically discrete units, each unit including a predetermined amount of the compound (e.g., a compound of Formula (I) or a hydrate, solvate, tautomer, or pharmaceutically acceptable salt thereof), sufficient to produce the desired effect. It will be appreciated that the parameters of a unit dosage form will depend on the particular agent and the effect to be achieved.

Additional Embodiments.

Embodiments include embodiment 1 to 278 following.

Embodiment 1. A composition, comprising:
(i) a compound of structural Formula (I):

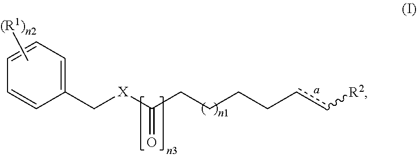

or an isotopic variant thereof; or a metabolite, pharmaceutically acceptable salt, solvate, or hydrate thereof,
   wherein:
[symbol] is a single bond or double bond;
n1 and n2 are independently an integer from 0 to 2;
n3 is 0 or 1;
X is O, NH, or S;
R¹ is independently hydrogen, halogen, −OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
(ii) at least one additional therapeutic agent selected from:
   a fatty acid amide hydrolase (FAAH) receptor inhibitor, and
   a specialized pro-resolving mediator (SPM), or a combination thereof; and
(iii) at least one excipient.

Embodiment 2. A composition, comprising:
(i) a compound of structural Formula (I):

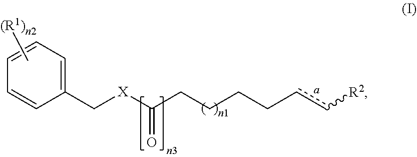

or an isotopic variant thereof; or a metabolite, pharmaceutically acceptable salt, solvate, or hydrate thereof,
   wherein:
[symbol] is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
(ii) a transient receptor potential cation channel subfamily V member 1 (TrpV1) modulator; and
(iii) at least one excipient,
   wherein when the compound of structural Formula (I), or an isotopic variant thereof; or a metabolite, pharmaceutically acceptable salt, solvate, or hydrate thereof, is capsaicin, then the capsaicin is present in the composition in an amount of greater than about 40% (w/v).

Embodiment 3. The composition of embodiment 2, wherein the capsaicin is present in the composition in an amount of from about 40% (w/v) to about 50% (w/v).

Embodiment 4. A composition, comprising:
(i) a compound of structural Formula (I):

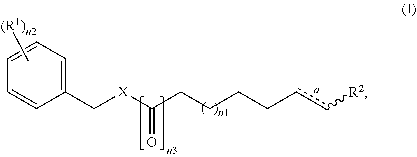

or an isotopic variant thereof; or a metabolite, pharmaceutically acceptable salt, solvate, or hydrate thereof,
   wherein:
[symbol] is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
(ii) a TrpV1 modulator; and
(iii) at least one excipient,
wherein the compound of Formula (I), isotopic variant thereof; or a metabolite, pharmaceutically acceptable salt, solvate, or hydrate thereof, is not capsaicin; or
wherein the TrpV1 modulator is not a fatty acid.

Embodiment 5. The composition of any one of embodiments 1-4, wherein the at least one TrpV1 modulator is a TrpV1 antagonist, a TrpV1 competitive agonist, or combination thereof.

Embodiment 6. The composition of embodiment 4, wherein the at least one TrpV1 modulator is a triglyceride or spilanthol.

Embodiment 7. The composition of any one of embodiments 1-3 or 5, wherein the at least one TrpV1 modulator is a fatty acid, triglyceride, or spilanthol.

Embodiment 8. The composition of embodiment 7, wherein the fatty acid is a polyunsaturated fatty acid (PUFA).

Embodiment 9. The composition of embodiment 8, wherein the PUFA is anandamide.

Embodiment 10. The composition of embodiment 7, wherein the fatty acid is an omega-9 fatty acid.

Embodiment 11. The composition of embodiment 10, wherein the fatty acid is oleic acid or erucic acid.

Embodiment 12. The composition of embodiment 1, wherein the at least one additional therapeutic agent is an FAAH receptor inhibitor.

Embodiment 13. The composition of embodiment 1, wherein the at least one additional therapeutic agent is an SPM.

Embodiment 14. The composition of embodiment 13, wherein the SPM is maresin.

Embodiment 15. The composition of any one of embodiments 1-14, wherein the pharmaceutical composition is in a form for oral dosing or administration.

Embodiment 16. The composition of embodiment 15, wherein the form is a suspension or a solution.

Embodiment 17. The composition of embodiment 15, wherein the form is a solid dosage form.

Embodiment 18. The composition of embodiment 17, wherein the form is a tablet or a capsule.

Embodiment 19. The composition of any one of embodiments 1-14, wherein the pharmaceutical composition is in a form for inhalation, nebulization, or nasal delivery.

Embodiment 20. The composition of embodiment 19, wherein the pharmaceutical composition is in a form for inhalation administration or dosing by vaporizer.

Embodiment 21. The composition of any one of embodiments 1-14, wherein the pharmaceutical composition is in a form for parenteral administration or dosing.

Embodiment 22. The composition of embodiment 21, wherein the parenteral administration or dosing is injection, infusion, or implantation.

Embodiment 23. The composition of embodiment 22, wherein the injection is intravenous, intramuscular, subcutaneous, or intradermal.

Embodiment 24. The composition of any one of embodiments 1-14, wherein the pharmaceutical composition is in a form for dosing or administration by suppository.

Embodiment 25. The composition of any one of embodiments 1-14, wherein the pharmaceutical composition is in a form for cutaneous or transdermal administration.

Embodiment 26. The composition of any one of embodiments 1-14, wherein the pharmaceutical composition is in a form for sublingual or buccal administration.

Embodiment 27. The composition of any one of embodiments 4-26, wherein the compound of Formula (I), or an isotopic variant thereof; or a metabolite, pharmaceutically acceptable salt, solvate, or hydrate thereof is present in the composition in an amount of greater than about 10% (w/v).

Embodiment 28. The composition of any one of embodiments 4-27, wherein the compound of Formula (I), or an isotopic variant thereof; or a metabolite, pharmaceutically acceptable salt, solvate, or hydrate thereof is present in the composition in an amount of greater than about 20% (w/v).

Embodiment 29. The composition of any one of embodiments 4-28, wherein the compound of Formula (I), or an isotopic variant thereof; or a metabolite, pharmaceutically acceptable salt, solvate, or hydrate thereof is present in the composition in an amount of greater than about 30% (w/v).

Embodiment 30. The composition of any one of embodiments 4-29, wherein the compound of Formula (I), or an isotopic variant thereof; or a metabolite, pharmaceutically acceptable salt, solvate, or hydrate thereof is present in the composition in an amount of greater than about 40% (w/v).

Embodiment 31. The composition of any one of embodiments 4-26, wherein the compound of Formula (I), or an isotopic variant thereof; or a metabolite, pharmaceutically acceptable salt, solvate, or hydrate thereof is present in the composition in an amount of from about 40% (w/v) to about 50% (w/v).

Embodiment 32. A composition, comprising: (i) a compound of structural Formula (I):

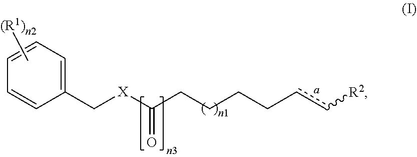

or an isotopic variant thereof; or a metabolite, pharmaceutically acceptable salt, solvate, or hydrate thereof,
   wherein: [symbol] is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
(ii) a TrpV1 modulator; and
(iii) at least one excipient,
   wherein the composition is formulated for systemic dosing or delivery.

Embodiment 33. The composition of embodiment 32, wherein the dosing or delivery is by parenteral or enteral administration.

Embodiment 34. The composition of embodiment 33, wherein the composition is formulated as a liquid or a solid.

Embodiment 35. The composition of embodiment 34, wherein the composition is formulated as a solid.

Embodiment 36. The composition of embodiment 35, wherein the solid is a tablet, a capsule, a suppository, strip, or an implant.

Embodiment 37. The composition of embodiment 36, wherein the tablet is a dissolvable tablet or a bilayer tablet.

Embodiment 38. The composition of embodiment 36, wherein the strip is a dissolvable strip.

Embodiment 39. The composition of embodiment 34, wherein the composition is formulated as a liquid.

Embodiment 40. The composition of embodiment 39, wherein the liquid is a solution, a suspension, an emulsion, a syrup, an elixir, a tincture, an ointment, a soft gel, an enema, foam, cream, lotion, or a gel.

Embodiment 41. The composition of embodiment 33, wherein the composition is formulated for dosing or delivery by enteral administration.

Embodiment 42. The composition of embodiment 41, wherein the enteral administration is oral or rectal administration.

Embodiment 43. The composition of embodiment 33, wherein the composition is formulated for dosing or delivery by parenteral administration.

Embodiment 44. The composition of embodiment 43, wherein the parenteral administration is sublingual administration, buccal administration, administration by injection, infusion, implantation, or inhalation.

Embodiment 45. A solid composition, comprising:
(i) a compound of structural Formula (I):

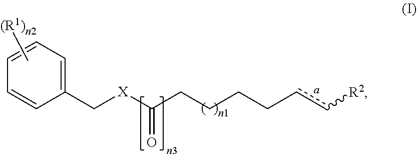

or an isotopic variant thereof; or a metabolite, pharmaceutically acceptable salt, solvate, or hydrate thereof,
   wherein:

[symbol] is a single bond or double bond;

n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
   (ii) a TrpV1 modulator; and
   (iii) at least one excipient.

Embodiment 46. The composition of embodiment 45, wherein the solid is a tablet, a capsule, a suppository, strip, or an implant.

Embodiment 47. The composition of embodiment 46, wherein the tablet is a dissolvable tablet or a bilayer tablet.

Embodiment 48. The composition of embodiment 46, wherein the strip is a dissolvable strip

Embodiment 49. A composition, comprising: (i) a compound of structural Formula (I):

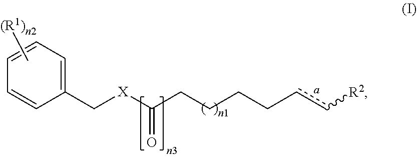

or an isotopic variant thereof; or a metabolite, pharmaceutically acceptable salt, solvate, or hydrate thereof,
   wherein:
[symbol] is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
(ii) a TrpV1 modulator; and
(iii) at least one excipient,
   wherein the compound of Formula (I), or an isotopic variant thereof; or a metabolite, pharmaceutically acceptable salt, solvate, or hydrate thereof, and the TrpV1 modulator are released sequentially in any order or have different drug release profiles.

Embodiment 50. The composition of embodiment 48, wherein the compound of Formula (I), or an isotopic variant thereof; or a metabolite, pharmaceutically acceptable salt, solvate, or hydrate thereof, is released before the at least one additional therapeutic agent.

Embodiment 51. The composition of embodiment 48, wherein the compound of Formula (I), or an isotopic variant thereof; or a metabolite, pharmaceutically acceptable salt, solvate, or hydrate thereof, is released after the at least one additional therapeutic agent.

Embodiment 52. The composition of any one of embodiments 48-50, wherein the solid is a tablet, a capsule, a suppository, strip, or an implant.

Embodiment 53. The composition of embodiment 51, wherein the tablet is a dissolvable tablet or a bilayer tablet.

Embodiment 54. The composition of embodiment 51, wherein the strip is a dissolvable strip.

Embodiment 55. The composition of any one of embodiments 33-53, wherein the compound of Formula (I), or an isotopic variant thereof; or a metabolite, pharmaceutically acceptable salt, solvate, or hydrate thereof is present in the composition in an amount of greater than about 10% (w/v).

Embodiment 56. The composition of any one of embodiments 33-54, wherein the compound of Formula (I), or an isotopic variant thereof; or a metabolite, pharmaceutically acceptable salt, solvate, or hydrate thereof is present in the composition in an amount of greater than about 20% (w/v).

Embodiment 57. The composition of any one of embodiments 33-54, wherein the compound of Formula (I), or an isotopic variant thereof; or a metabolite, pharmaceutically acceptable salt, solvate, or hydrate thereof is present in the composition in an amount of greater than about 30% (w/v).

Embodiment 58. The composition of any one of embodiments 33-54, wherein the compound of Formula (I), or an isotopic variant thereof; or a metabolite, pharmaceutically acceptable salt, solvate, or hydrate thereof is present in the composition in an amount of greater than about 40% (w/v).

Embodiment 59. The composition of any one of embodiments 33-53, wherein the compound of Formula (I), or an isotopic variant thereof; or a metabolite, pharmaceutically acceptable salt, solvate, or hydrate thereof is present in the composition in an amount of from about 40% (w/v) to about 50% (w/v).

Embodiment 60. The composition of any one of embodiments 1-58, wherein the composition is a modified-release dosage form, a sustained-release dosage form, or a controlled-release dosage form.

Embodiment 61. The composition of embodiment 59, wherein the composition is in modified-release dosage form.

Embodiment 62. The composition of embodiment 51, wherein the modified-release dosage form is a delayed-release dosage form, an extended release dosage form, or a targeted release dosage form.

Embodiment 63. The composition of any one of embodiments 2-11 or 16-61, wherein the ratio of TrpV1 modulator to a compound of Formula (I), or an isotopic variant thereof; or a metabolite, pharmaceutically acceptable salt, solvate, or hydrate thereof, is about 1:1.

Embodiment 64. The composition of any one of embodiments 12-13 or 16-61, wherein the ratio of FAAH receptor inhibitor to a compound of Formula (I), or an isotopic variant thereof; or a metabolite, pharmaceutically acceptable salt, solvate, or hydrate thereof, is about 1:1.

Embodiment 65. The composition of any one of embodiments 14-15 or 16-61, wherein the ratio of SPM to a compound of Formula (I), or an isotopic variant thereof; or a metabolite, pharmaceutically acceptable salt, solvate, or hydrate thereof, is about 1:1.

Embodiment 66. The composition of any one of embodiments 1-64, wherein the composition is formulated as a pharmaceutical composition.

Embodiment 67. The pharmaceutical composition of embodiment 65, wherein the at least one excipient is at least one pharmaceutically acceptable excipient.

Embodiment 68. The pharmaceutical composition of embodiment 66, wherein the compound of structural Formula (I) has structural Formula (I-A):

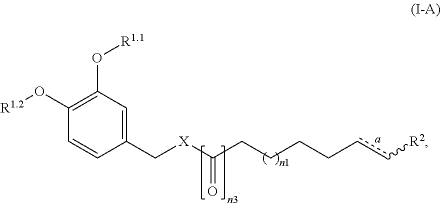

or an isotopic variant thereof; or a metabolite, pharmaceutically acceptable salt, solvate, or hydrate thereof,
   wherein:
R^{1.1} is hydrogen, halogen, —OR^{1.1A}, substituted or unsubstituted alkyl, or substituted or unsubstituted heteroalkyl;
R^{1.2} is hydrogen, halogen, —OR^{1.2A}, substituted or unsubstituted alkyl, or substituted or unsubstituted heteroalkyl; and
R^{1.1A} and R^{1.2A} are independently hydrogen, —CF₃, —l₃, —CBr₃, —CI₃, —COOH, —CONH₂, substituted or unsubstituted alkyl, or substituted or unsubstituted heteroalkyl.

Embodiment 69. The pharmaceutical composition of embodiment 67, wherein:
R^{1.1} is CH₃; and
R^{1.2} is OH.

Embodiment 70. The pharmaceutical composition of embodiment 67 or 68, wherein X is O.

Embodiment 71. The pharmaceutical composition of embodiment 67 or 68, wherein X is NH.

Embodiment 72. The pharmaceutical composition of any one of embodiments 67-70, wherein [symbol] is a double bond.

Embodiment 73. The pharmaceutical composition of any one of embodiments 67-70, wherein [symbol] is a single bond.

Embodiment 74. The pharmaceutical composition of any one of embodiments 67-72, wherein n1 is 2.

Embodiment 75. The pharmaceutical composition of any one of embodiments 67-72, wherein n1 is 1.

Embodiment 76. The pharmaceutical composition of any one of embodiments 67-72, wherein n1 is 0.

Embodiment 77. The pharmaceutical composition of any one of embodiments 67-74, wherein R² is unsubstituted alkyl.

Embodiment 78. The pharmaceutical composition of any one of embodiments 67-75, wherein R² is —CH(CH₃)₂ or —CH₃.

Embodiment 79. The pharmaceutical composition of embodiment 77, wherein the compound is at least one of:

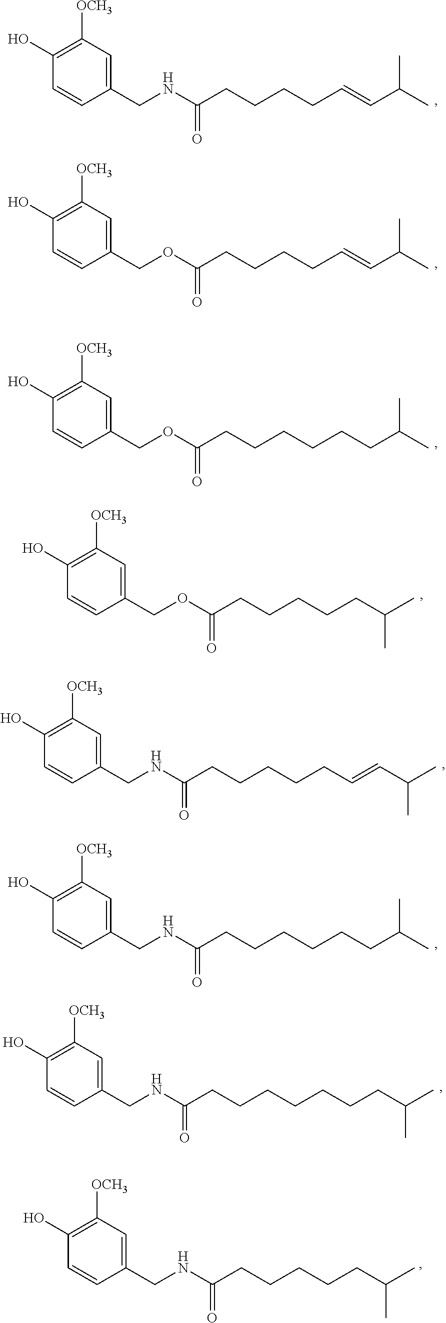

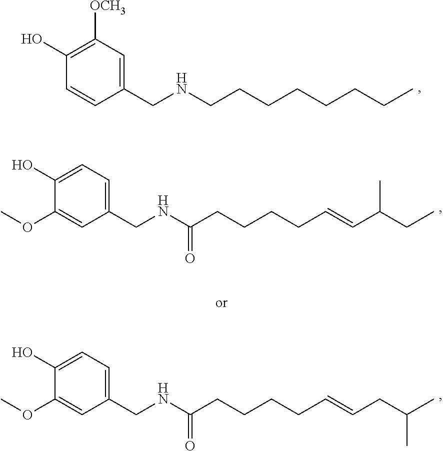

or an isotopic variant thereof; or a metabolite, pharmaceutically acceptable salt, solvate, or hydrate thereof.

Embodiment 80. The pharmaceutical composition of embodiment 78, wherein the compound is:

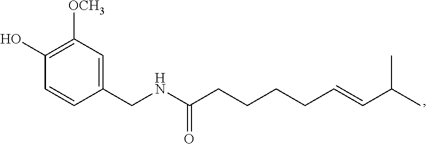 [SMILES: COc1cc(CNC(=O)CCCC/C=C/C(C)C)ccc1O]

or an isotopic variant thereof; or a metabolite, pharmaceutically acceptable salt, solvate, or hydrate thereof

Embodiment 81. A method of treating or preventing a capsaicin-responsive disease or disorder, comprising administering to a subject in need thereof the pharmaceutical composition of any one of embodiments 1-79.

Embodiment 82. The method of embodiment 80, wherein the disease or disorder is a cell proliferative disease, obesity, diabetes, a bacterial infection, a cardiovascular disease, a neurodegenerative disease or disorder, an inflammatory disease or disorder, a comorbidity, an autoimmune disease, hypercholesterolemia or a mood disorder.

Embodiment 83. The method of embodiment 81, wherein the mood disorder is depression.

Embodiment 84. The method of embodiment 81, wherein the autoimmune disorder is rheumatoid arthritis.

Embodiment 85. The method of embodiment 81, wherein the cell proliferative disease is a cancer.

Embodiment 86. The method of embodiment 81, wherein the disorder is diabetes or obesity.

Embodiment 87. The method of embodiment 81, wherein the comorbidity is chronic kidney disease, stroke, congestive heart failure, dementia, schizophrenia, hepatitis, autism spectrum disorder, HIV, or a combination thereof.

Embodiment 88. A supplement, comprising:
(i) a compound of structural Formula (I):

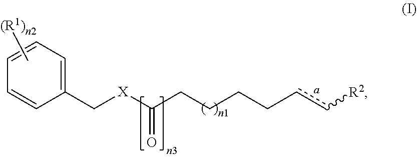

or an isotopic variant thereof; or a metabolite, pharmaceutically acceptable salt, solvate, or hydrate thereof,
   wherein:
[symbol] is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
(ii) at least one additional therapeutic agent selected from:
   a fatty acid amide hydrolase (FAAH) receptor inhibitor, and
   a specialized pro-resolving mediator (SPM), or a combination thereof; and
(iii) at least one excipient.

Embodiment 89. The supplement of embodiment 87, wherein the compound of structural Formula (I) has structural Formula (I-A):

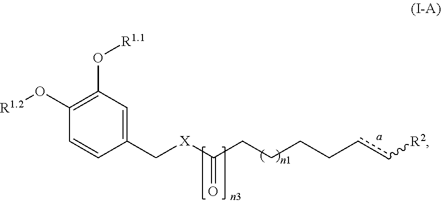

or an isotopic variant thereof; or a metabolite, pharmaceutically acceptable salt, solvate, or hydrate thereof,
   wherein:
R^{1.1} is hydrogen, halogen, —OR^{1.1A}, substituted or unsubstituted alkyl, or substituted or unsubstituted heteroalkyl;
R^{1.2} is hydrogen, halogen, —OR^{1.2A}, substituted or unsubstituted alkyl, or substituted or unsubstituted heteroalkyl; and
^{1.1A} and R^{1.2A} are independently hydrogen, —CF₃, —CCl₃, —CBr₃, —CI₃, —COOH, —CONH₂, substituted or unsubstituted alkyl, or substituted or unsubstituted heteroalkyl.

Embodiment 90. The supplement of embodiment 88, wherein:
R^{1.1} is CH₃; and
R^{1.2} is OH.

Embodiment 91. The supplement of any one of embodiments 87-89, wherein X is O.

Embodiment 92. The supplement of any one of embodiments 87-89, wherein X is NH.

Embodiment 93. The supplement of any one of embodiments 87-91, wherein [symbol] is a double bond.

Embodiment 94. The supplement of any one of embodiments 87-91, wherein [symbol] is a single bond.

Embodiment 95. The supplement of any one of embodiments 87-93, wherein n1 is 2.

Embodiment 96. The supplement of any one of embodiments 87-93, wherein n1 is 1.

Embodiment 97. The supplement of any one of embodiments 87-93, wherein n1 is 0.

Embodiment 98. The supplement of any one of embodiments 87-96, wherein R² is unsubstituted alkyl.

Embodiment 99. The supplement of any one of embodiments 87-97, wherein R² is —CH(CH₃)₂ or —CH₃.

Embodiment 100. The supplement of embodiment 87, wherein the compound is at least one of:

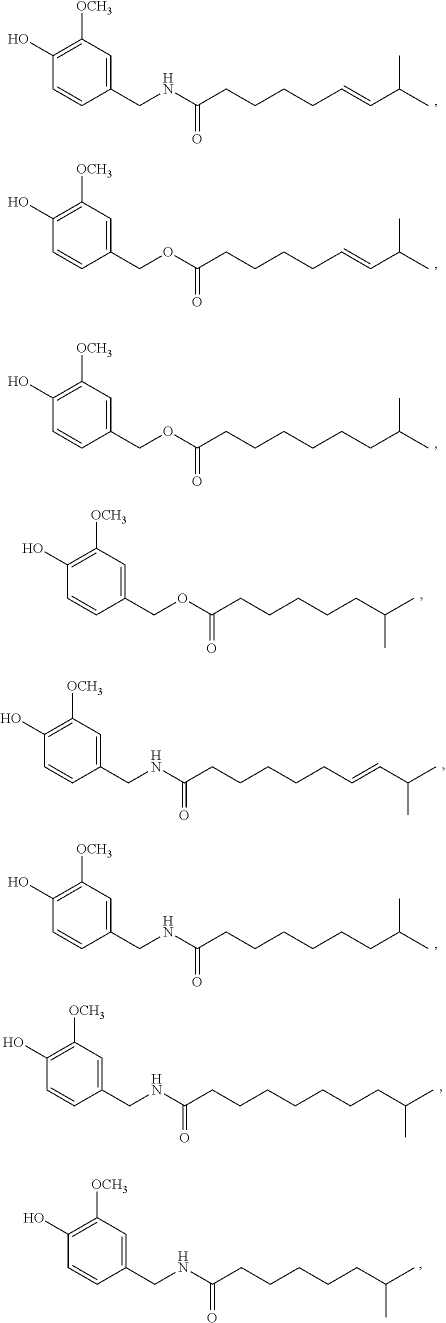

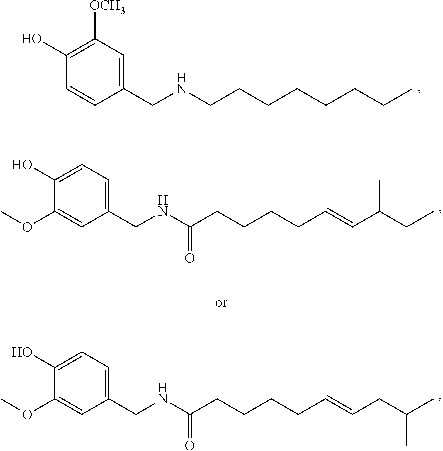

or an isotopic variant thereof; or a metabolite, pharmaceutically acceptable salt, solvate, or hydrate thereof.

Embodiment 101. The supplement of embodiment 99, wherein the compound is:

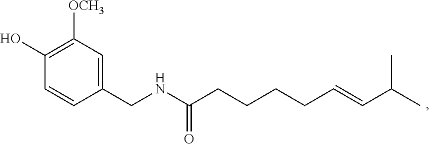 [SMILES: COc1cc(CNC(=O)CCCC/C=C/C(C)C)ccc1O]

or an isotopic variant thereof; or a metabolite, pharmaceutically acceptable salt, solvate, or hydrate thereof.

Embodiment 102. The supplement of any one of embodiments 87-100, wherein the at least one additional therapeutic agent is a TrpV1 modulator.

Embodiment 103. The supplement of any one of embodiments 87-101, wherein the at least one TrpV1 modulator is a TrpV1 antagonist, a TrpV1 competitive agonist, or combination thereof

Embodiment 104. The supplement of any one of embodiments 87-102, wherein the at least one TrpV1 modulator is a fatty acid, triglyceride, or spilanthol.

Embodiment 105. The supplement of embodiment 103, wherein the fatty acid is a polyunsaturated fatty acid (PUFA).

Embodiment 106. The supplement of embodiment 104, wherein the PUFA is anandamide.

Embodiment 107. The supplement of embodiment 103, wherein the fatty acid is an omega-9 fatty acid.

Embodiment 108. The supplement of embodiment 106, wherein the fatty acid is oleic acid or erucic acid.

Embodiment 109. The supplement of any one of embodiments 87-100, wherein the at least one additional therapeutic agent is an FAAH receptor inhibitor.

Embodiment 110. The supplement of embodiment 108, wherein the FAAH receptor inhibitor is SSR411298.

Embodiment 111. The supplement of any one of embodiments 87-100, wherein the at least one additional therapeutic agent is an SPM.

Embodiment 112. The supplement of embodiment 109, wherein the SPM is maresin.

Embodiment 113. The supplement of any one of embodiments 87-111, wherein the supplement is in a form for oral dosing or administration.

Embodiment 114. The supplement of embodiment 112, wherein the form is a suspension or a solution.

Embodiment 115. The supplement of embodiment 113, wherein the form is a solid dosage form.

Embodiment 116. The supplement of embodiment 114, wherein the form is a tablet or a capsule.

Embodiment 117. The supplement of any one of embodiments 87-111, wherein the supplement is in a form for inhalation, nebulization, or nasal delivery.

Embodiment 118. The supplement of embodiment 116, wherein the supplement is in a form for inhalation administration or dosing by vaporizer.

Embodiment 119. The supplement of any one of embodiments 87-111, wherein the supplement is in a form for parenteral administration or dosing.

Embodiment 120. The supplement of embodiment 118, wherein the administration or dosing is parenteral injection.

Embodiment 121. The supplement of embodiment 120, wherein the injection is intravenous, intramuscular, subcutaneous, or intradermal.

Embodiment 122. The supplement of any one of embodiments 87-111, wherein the supplement is in a form for dosing or administration by suppository.

Embodiment 123. The supplement of any one of embodiments 87-111, wherein the supplement is in a form for cutaneous or transdermal administration.

Embodiment 124. The supplement of any one of embodiments 87-111, wherein the supplement is in a form for sublingual or buccal administration.

Embodiment 125. The supplement of any one of embodiments 87-123, wherein the compound of Formula (I), or an isotopic variant thereof; or a metabolite, pharmaceutically acceptable salt, solvate, or hydrate thereof is present in the composition in an amount of greater than about 10% (w/v).

Embodiment 126. The supplement of any one of embodiments 87-124, wherein the compound of Formula (I), or an isotopic variant thereof; or a metabolite, pharmaceutically acceptable salt, solvate, or hydrate thereof is present in the composition in an amount of greater than about 20% (w/v).

Embodiment 127. The supplement of any one of embodiments 87-125, wherein the compound of Formula (I), or an isotopic variant thereof; or a metabolite, pharmaceutically acceptable salt, solvate, or hydrate thereof is present in the composition in an amount of greater than about 30% (w/v).

Embodiment 128. The supplement of any one of embodiments 87-126, wherein the compound of Formula (I), or an isotopic variant thereof; or a metabolite, pharmaceutically acceptable salt, solvate, or hydrate thereof is present in the composition in an amount of greater than about 40% (w/v).

Embodiment 129. The supplement of any one of embodiments 87-126, wherein the compound of Formula (I), or an isotopic variant thereof; or a metabolite, pharmaceutically acceptable salt, solvate, or hydrate thereof is present in the composition in an amount of from about 40% (w/v) to about 50% (w/v).

Embodiment 130. The supplement of any one of embodiments 87-128 for use in the treatment of a capsaicin-responsive disease or disorder.

Embodiment 131. The supplement of embodiment 129, wherein the disease or disorder is a cell proliferative disease, obesity, diabetes, a bacterial infection, a cardiovascular disease, a neurodegenerative disease or disorder, an inflammatory disease or disorder, a comorbidity, an autoimmune disease, hypercholesterolemia or a mood disorder.

Embodiment 132. The supplement of embodiment 130, wherein the mood disorder is depression.

Embodiment 133. The supplement of embodiment 130, wherein the autoimmune disorder is rheumatoid arthritis.

Embodiment 134. The supplement of embodiment 130, wherein the cell proliferative disease is a cancer.

Embodiment 135. The supplement of embodiment 130, wherein the disorder is diabetes or obesity.

Embodiment 136. A method of treating or preventing a capsaicin-responsive disease or disorder, comprising administering to a subject in need thereof:
(i) a compound of structural Formula (I):

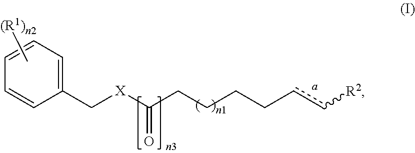

or an isotopic variant thereof; or a metabolite, pharmaceutically acceptable salt, solvate, or hydrate thereof,
   wherein:
[symbol] is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl; and
(ii) at least one additional therapeutic agent selected from:
   a transient receptor potential cation channel subfamily V member 1 (TrpV1) modulator,
   fatty acid amide hydrolase (FAAH) receptor inhibitor; and
   a specialized pro-resolving mediator (SPM), or a combination thereof,
      wherein when the TrpV1 modulator is a fatty acid, then the compound of structural Formula (I) or an isotopic variant thereof; or a metabolite, pharmaceutically acceptable salt, solvate, or hydrate thereof, and the TrpV1 modulator are each individually formulated.

Embodiment 137. The method of embodiment 135, wherein the compound of structural Formula (I) has structural Formula (I-A):

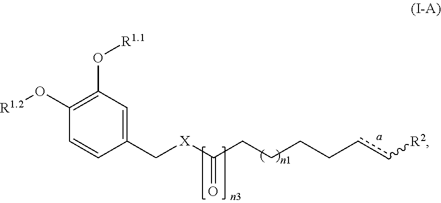

or an isotopic variant thereof; or a metabolite, pharmaceutically acceptable salt, solvate, or hydrate thereof,
   wherein:
R^{1.2} is hydrogen, halogen, —OR^{1.2A}, substituted or unsubstituted alkyl, or substituted or unsubstituted heteroalkyl;
R^{1.1A} is hydrogen, halogen, —OR^{1.2A}, substituted or unsubstituted alkyl, or substituted or unsubstituted heteroalkyl; and
R^{1.1A} and R^{1.2A} are independently hydrogen, —CF₃, —CCl₃, —CBr₃, —CI₃, —COOH, —CONH₂, substituted or unsubstituted alkyl, or substituted or unsubstituted heteroalkyl.

Embodiment 138. The method of embodiment 136, wherein:
R^{1.1} is CH₃; and
R^{1.2} is OH.

Embodiment 139. The method of any one of embodiments 135-137, wherein Xis O.

Embodiment 140. The method of any one of embodiments 135-137, wherein Xis NH.

Embodiment 141. The method of any one of embodiments 135-139, wherein [symbol] is a double bond.

Embodiment 142. The method of any one of embodiments 135-139, wherein [symbol] is a single bond.

Embodiment 143. The method of any one of embodiments 135-141, wherein n1 is 2.

Embodiment 144. The method of any one of embodiments 135-141, wherein n1 is 1.

Embodiment 145. The method of any one of embodiments 135-141, wherein n1 is 0.

Embodiment 146. The method of any one of embodiments 135-144, wherein R² is unsubstituted alkyl.

Embodiment 147. The method of any one of embodiments 135-145, wherein R² is —CH(CH₃)₂ or —CH₃.

Embodiment 148. The method of embodiment 135, wherein the compound is at least one of:

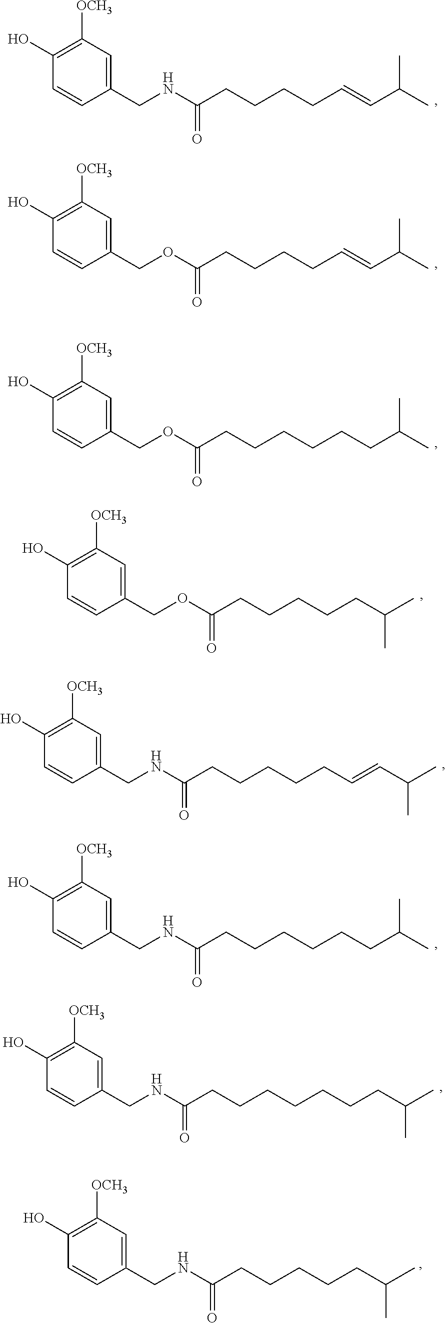

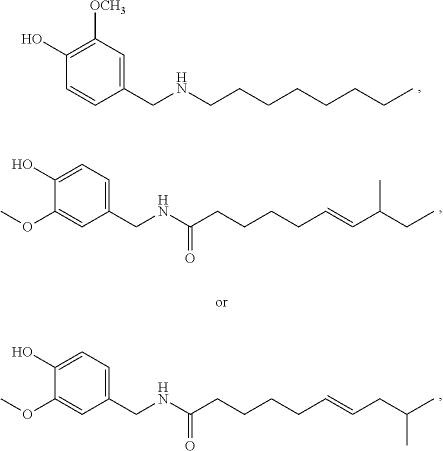

or an isotopic variant thereof; or a metabolite, pharmaceutically acceptable salt, solvate, or hydrate thereof.

Embodiment 149. The method of embodiment 147, wherein the compound is:

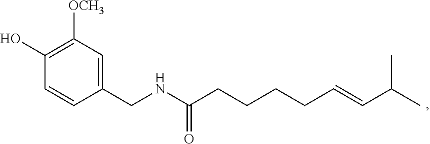 [SMILES: COc1cc(CNC(=O)CCCC/C=C/C(C)C)ccc1O]

or an isotopic variant thereof; or a metabolite, pharmaceutically acceptable salt, solvate, or hydrate thereof.

Embodiment 150. The method of any one of embodiments 135-148, wherein the at least one additional therapeutic agent is a TrpV1 modulator.

Embodiment 151. The method of any one of embodiments 135-149, wherein the TrpV1 modulator is a TrpV1 antagonist, a TrpV1 competitive agonist, or combination thereof.

Embodiment 152. The method of any one of embodiments 135-150, wherein the TrpV1 modulator is a fatty acid triglyceride, or spilanthol.

Embodiment 153. The method of embodiment 151, wherein the fatty acid is a polyunsaturated fatty acid (PUFA).

Embodiment 154. The method of embodiment 152, wherein the PUFA is anandamide

Embodiment 155. The method of embodiment 151, wherein the fatty acid is an omega-9 fatty acid.

Embodiment 156. The method of embodiment 154, wherein the fatty acid is oleic acid or erucic acid.

Embodiment 157. The method of any one of embodiments 135-148, wherein the at least one additional therapeutic agent is a FAAH receptor inhibitor.

Embodiment 158. The method of embodiment 156, wherein the FAAH receptor inhibitor is SR411298.

Embodiment 159. The method of any one of embodiments 135-148, wherein the at least one additional therapeutic agent is an SPM.

Embodiment 160. The method of embodiment 158, wherein the SPM is maresin.

Embodiment 161. The method of any one of embodiments 135-159, wherein the compound of structural Formula (I) or an isotopic variant thereof; or a metabolite, pharmaceutically acceptable salt, solvate, or hydrate thereof, is administered orally.

Embodiment 162. The method of any one of embodiments 135-160, wherein the compound of structural Formula (I) or an isotopic variant thereof; or a metabolite, pharmaceutically acceptable salt, solvate, or hydrate thereof, is administered orally via a suspension or solution.

Embodiment 163. The method of any one of embodiments 135-160, wherein the compound of structural Formula (I) or an isotopic variant thereof; or a metabolite, pharmaceutically acceptable salt, solvate, or hydrate thereof, is administered orally via a solid dosage form.

Embodiment 164. The method of embodiment 162, wherein the form is a tablet or capsule.

Embodiment 165. The method of any one of embodiments 135-159, wherein the compound of structural Formula (I) or an isotopic variant thereof; or a metabolite, pharmaceutically acceptable salt, solvate, or hydrate thereof, is administered by inhalation, nebulization, or nasal delivery.

Embodiment 166. The method of embodiment 164, wherein the compound of structural Formula (I) or an isotopic variant thereof; or a metabolite, pharmaceutically acceptable salt, solvate, or hydrate thereof, is administered by inhalation via a vaporizer.

Embodiment 167. The method of any one of embodiments 135-159, wherein the compound of structural Formula (I) or an isotopic variant thereof; or a metabolite, pharmaceutically acceptable salt, solvate, or hydrate thereof, is administered parenterally.

Embodiment 168. The method of any one of embodiments 135-159, wherein the compound of structural Formula (I) or an isotopic variant thereof; or a metabolite, pharmaceutically acceptable salt, solvate, or hydrate thereof, is administered via parenteral injection, infusion, or implantation.

Embodiment 169. The method of embodiment 167, wherein the parenteral injection is intravenous, intramuscular, subcutaneous, or intradermal.

Embodiment 170. The method of any one of embodiments 135-159, wherein the compound of structural Formula (I) or an isotopic variant thereof; or a metabolite, pharmaceutically acceptable salt, solvate, or hydrate thereof, is administered via a suppository.

Embodiment 171. The method of any one of embodiments 135-159, wherein the compound of structural Formula (I) or an isotopic variant thereof; or a metabolite, pharmaceutically acceptable salt, solvate, or hydrate thereof, is administered cutaneous or transdermal delivery.

Embodiment 172. The method of any one of embodiments 135-159, wherein the compound of structural Formula (I) or an isotopic variant thereof; or a metabolite, pharmaceutically acceptable salt, solvate, or hydrate thereof, is administered by sublingual or buccal delivery.

Embodiment 173. The method of any one of embodiments 135-171, wherein the compound of Formula (I), or an isotopic variant thereof; or a metabolite, pharmaceutically acceptable salt, solvate, or hydrate thereof is present in the composition in an amount of greater than about 10% (w/v).

Embodiment 174. The method of any one of embodiments 135-172, wherein the compound of Formula (I), or an isotopic variant thereof; or a metabolite, pharmaceutically acceptable salt, solvate, or hydrate thereof is present in the composition in an amount of greater than about 20% (w/v).

Embodiment 175. The method of any one of embodiments 135-173, wherein the compound of Formula (I), or an isotopic variant thereof; or a metabolite, pharmaceutically acceptable salt, solvate, or hydrate thereof is present in the composition in an amount of greater than about 30% (w/v).

Embodiment 176. The method of any one of embodiments 135-174, wherein the compound of Formula (I), or an isotopic variant thereof; or a metabolite, pharmaceutically acceptable salt, solvate, or hydrate thereof is present in the composition in an amount of greater than about 40% (w/v).

Embodiment 177. The method of any one of embodiments 135-172, wherein the compound of Formula (I), or an isotopic variant thereof; or a metabolite, pharmaceutically acceptable salt, solvate, or hydrate thereof is present in the composition in an amount of from about 40% (w/v) to about 50% (w/v).

Embodiment 178. The method of any one of embodiments 135-176, wherein the ratio of the therapeutic agent to a compound of Formula (I), or an isotopic variant thereof; or a metabolite, pharmaceutically acceptable salt, solvate, or hydrate thereof, is about 1:1.

Embodiment 179. The method of embodiment any one of embodiments 135-177, wherein the disease or disorder is a cell proliferative disease, obesity, diabetes, a bacterial infection, a cardiovascular disease, a neurodegenerative disease or disorder, an inflammatory disease or disorder, a comorbidity, an autoimmune disease, hypercholesterolemia or a mood disorder .

Embodiment 180. The method of embodiment 178, wherein the mood disorder is depression.

Embodiment 181. The method of embodiment 179, wherein the autoimmune disorder is rheumatoid arthritis

Embodiment 182. The method of embodiment 179, wherein the cell proliferative disease is a cancer.

Embodiment 183. The method of embodiment 179, wherein the disorder is diabetes or obesity.

Embodiment 184. The method of any one of embodiments 135-182, wherein the compound of Formula (I), or a pharmaceutically acceptable salt thereof, and the at least one additional therapeutic agent are administered simultaneously, approximately simultaneously, or sequentially, in any order.

Embodiment 185. The method of embodiment 183, wherein the compound of Formula (I), or a pharmaceutically acceptable salt thereof, and the at least one additional therapeutic agent are administered simultaneously or approximately simultaneously.

Embodiment 186. The method of embodiment 183, wherein the compound of Formula (I), or a pharmaceutically acceptable salt thereof, and the at least one additional therapeutic agent are administered sequentially.

Embodiment 187. The method of embodiment 185, wherein the compound of Formula (I), or a pharmaceutically acceptable salt thereof, is released before the at least one additional therapeutic agent.

Embodiment 188. The method of embodiment 185, wherein the compound of Formula (I), or a pharmaceutically acceptable salt thereof, is released after the at least one additional therapeutic agent.

Embodiment 189. A composition, comprising: (i) a compound of structural Formula (I):

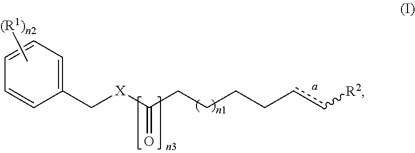

or an isotopic variant thereof; or a metabolite, pharmaceutically acceptable salt, solvate, or hydrate thereof,
   wherein:
[symbol] is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
(ii) a TrpV1 modulator; and
(iii) at least one excipient,
   wherein the composition is formulated for systemic dosing or delivery.

Embodiment 190. The composition of embodiment 189, wherein the dosing or delivery is by parenteral or enteral administration.

Embodiment 191. The composition of embodiment 190, wherein the composition is formulated as a liquid or a solid.

Embodiment 192. The composition of embodiment 191, wherein the composition is formulated as a solid.

Embodiment 193. The composition of embodiment 192, wherein the solid is a tablet, a capsule, a suppository, strip, or an implant.

Embodiment 194. The composition of embodiment 193, wherein the tablet is a dissolvable tablet or a bilayer tablet.

Embodiment 195. The composition of embodiment 193, wherein the strip is a dissolvable strip.

Embodiment 196. The composition of embodiment 190, wherein the composition is formulated as a liquid.

Embodiment 197. The composition of embodiment 196, wherein the liquid is a solution, a suspension, an emulsion, a syrup, an elixir, a tincture, an ointment, a soft gel, an enema, foam, cream, lotion, or a gel.

Embodiment 198. The composition of embodiment 190, wherein the composition is formulated for dosing or delivery by enteral administration.

Embodiment 199. The composition of embodiment 198, wherein the enteral administration is oral or rectal administration.

Embodiment 200. The composition of embodiment 190, wherein the composition is formulated for dosing or delivery by parenteral administration.

Embodiment 201. The composition of embodiment 200, wherein the parenteral administration is sublingual administration, buccal administration, administration by injection, infusion, implantation, or inhalation.

Embodiment 202. The composition of any one of embodiments 189-201, wherein the wherein the compound of Formula (I), or an isotopic variant thereof; or a metabolite, pharmaceutically acceptable salt, solvate, or hydrate thereof, and the TrpV1 modulator are released sequentially in any order or have different drug release profiles.

Embodiment 203. The composition of embodiment 202, wherein the compound of Formula (I), or an isotopic variant thereof; or a metabolite, pharmaceutically acceptable salt, solvate, or hydrate thereof, is released before the at least one additional therapeutic agent.

Embodiment 204. The composition of embodiment 202, wherein the compound of Formula (I), or an isotopic variant thereof; or a metabolite, pharmaceutically acceptable salt, solvate, or hydrate thereof, is released after the at least one additional therapeutic agent.

Embodiment 205. The composition of any one of embodiments 189-204, wherein the compound of Formula (I), or an isotopic variant thereof; or a metabolite, pharmaceutically acceptable salt, solvate, or hydrate thereof is present in the composition in an amount of greater than about 10% (w/v).

Embodiment 206. The composition of any one of embodiments 189-205, wherein the compound of Formula (I), or an isotopic variant thereof; or a metabolite, pharmaceutically acceptable salt, solvate, or hydrate thereof is present in the composition in an amount of greater than about 20% (w/v).

Embodiment 207. The composition of any one of embodiments 189-206, wherein the compound of Formula (I), or an isotopic variant thereof; or a metabolite, pharmaceutically acceptable salt, solvate, or hydrate thereof is present in the composition in an amount of greater than about 30% (w/v).

Embodiment 208. The composition of any one of embodiments 189-207, wherein the compound of Formula (I), or an isotopic variant thereof; or a metabolite, pharmaceutically acceptable salt, solvate, or hydrate thereof is present in the composition in an amount of greater than about 40% (w/v).

Embodiment 209. The composition of any one of embodiments 189-204, wherein the compound of Formula (I), or an isotopic variant thereof; or a metabolite, pharmaceutically acceptable salt, solvate, or hydrate thereof is present in the composition in an amount of from about 40% (w/v) to about 50% (w/v).

Embodiment 210. The composition of any one of embodiments 189-208, wherein the compound of Formula (I), isotopic variant thereof; or a metabolite, pharmaceutically acceptable salt, solvate, or hydrate thereof, is not capsaicin; or wherein the TrpV1 modulator is not a fatty acid.

Embodiment 211. The composition of any one of embodiments 189-210, wherein the at least one TrpV1 modulator is a TrpV1 antagonist, a TrpV1 competitive agonist, or combination thereof

Embodiment 212. The composition of any one of embodiments 189-211, wherein the at least one TrpV1 modulator is a fatty acid, triglyceride, or spilanthol.

Embodiment 213. The composition of embodiment 212, wherein the at least one TrpV1 modulator is a triglyceride or spilanthol.

Embodiment 214. The composition of embodiment 212, wherein the fatty acid is a polyunsaturated fatty acid (PUFA).

Embodiment 215. The composition of embodiment 214, wherein the PUFA is anandamide.

Embodiment 216. The composition of embodiment 212, wherein the fatty acid is an omega-9 fatty acid.

Embodiment 217. The composition of embodiment 216, wherein the fatty acid is oleic acid or erucic acid.

Embodiment 221. The composition of any one of embodiments 189-217, wherein the pharmaceutical composition is in a form for oral dosing or administration.

Embodiment 222. The composition of any one of embodiments 189-217, wherein the pharmaceutical composition is in a form for inhalation, nebulization, or nasal delivery.

Embodiment 223. The composition of embodiment 222, wherein the pharmaceutical composition is in a form for inhalation administration or dosing by vaporizer.

Embodiment 224. The composition of any one of embodiments 189-217, wherein the pharmaceutical composition is in a form for inhalation administration or dosing by vaporizer.

Embodiment 225. The composition of embodiment 224, wherein the injection is intravenous, intramuscular, subcutaneous, or intradermal.

Embodiment 226. The composition of any one of embodiments 189-217, wherein the pharmaceutical composition is in a form for dosing or administration by suppository.

Embodiment 227. The composition of any one of embodiments 189-217, wherein the pharmaceutical composition is in a form for cutaneous or transdermal administration.

Embodiment 228. The composition of any one of embodiments 189-217, wherein the pharmaceutical composition is in a form for sublingual or buccal administration.

Embodiment 229. The composition of any one of embodiments 189-228, wherein the composition is a modified-release dosage form, a sustained-release dosage form, or a controlled-release dosage form.

Embodiment 230. The composition of embodiment 229, wherein the composition is in modified-release dosage form.

Embodiment 231. The composition of embodiment 230, wherein the modified-release dosage form is a delayed-release dosage form, an extended release dosage form, or a targeted release dosage form.

Embodiment 232. The composition of any one of embodiments 189-231, wherein the ratio of TrpV1 modulator to a compound of Formula (I), or an isotopic variant thereof; or a metabolite, pharmaceutically acceptable salt, solvate, or hydrate thereof, is about 1:1.

Embodiment 233. The composition of any one of embodiments 189-232, wherein the composition is formulated as a pharmaceutical composition.

Embodiment 234. The pharmaceutical composition of embodiment 233, wherein the at least one excipient is at least one pharmaceutically acceptable excipient.

Embodiment 235. The pharmaceutical composition of any one of embodiments 189-234, wherein the compound of structural Formula (I) has structural Formula (I-A):

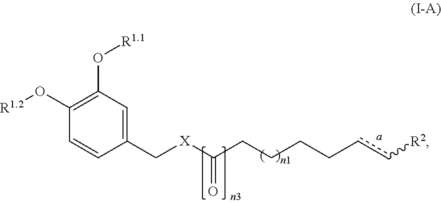

or an isotopic variant thereof; or a metabolite, pharmaceutically acceptable salt, solvate, or hydrate thereof,
   wherein:
R^{1.1} is hydrogen, halogen, —OR^{1.1A}, substituted or unsubstituted alkyl, or substituted or unsubstituted heteroalkyl;
R^{1.2} is hydrogen, halogen, —OR^{1.2A}, substituted or unsubstituted alkyl, or substituted or unsubstituted heteroalkyl; and
R^{1.1A} and R^{1.2A} are independently hydrogen, —CF₃, —CCl₃, —CBr₃, —CI₃, —COOH, —CONH₂, substituted or unsubstituted alkyl, or substituted or unsubstituted heteroalkyl.

Embodiment 236. The pharmaceutical composition of embodiment 235, wherein:
R^{1.1} is CH₃; and
R^{1.2} is OH.

Embodiment 237. The pharmaceutical composition of embodiment 235 or 236, wherein X is O.

Embodiment 238. The pharmaceutical composition of embodiment 235 or 236, wherein X is NH.

Embodiment 239. The pharmaceutical composition of any one of embodiments 189-238, wherein [symbol] is a double bond.

Embodiment 240. The pharmaceutical composition of any one of embodiments 189-238, wherein [symbol] is a single bond.

Embodiment 241. The pharmaceutical composition of any one of embodiments 189-239, wherein n1 is 2.

Embodiment 242. The pharmaceutical composition of any one of embodiments 189-239, wherein n1 is 1.

Embodiment 243. The pharmaceutical composition of any one of embodiments 189-239, wherein n1 is 0.

Embodiment 244. The pharmaceutical composition of any one of embodiments 189-243, wherein R² is unsubstituted alkyl.

Embodiment 245. The pharmaceutical composition of any one of embodiments 189-244, wherein R² is —CH(CH₃)₂ or —CH₃.

Embodiment 246. The pharmaceutical composition of embodiment 189, wherein the compound is at least one of:

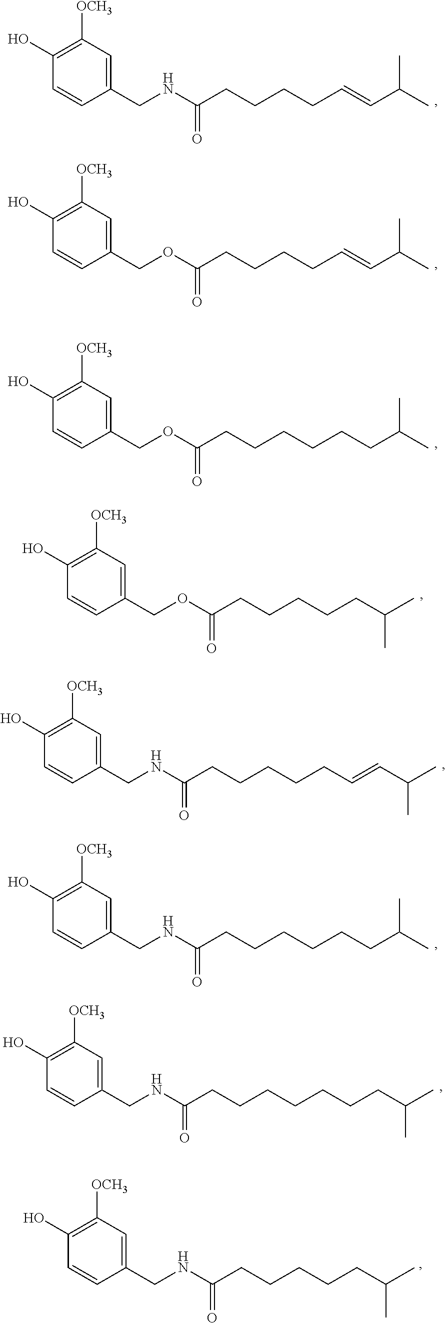

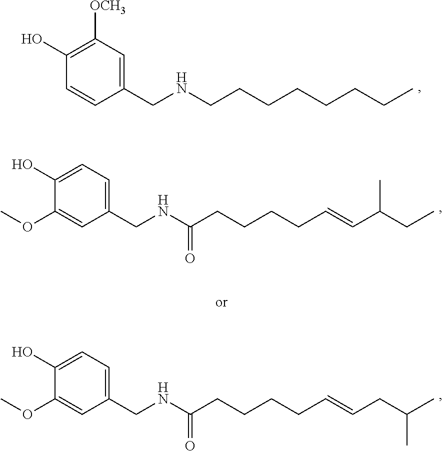

or an isotopic variant thereof; or a metabolite, pharmaceutically acceptable salt, solvate, or hydrate thereof.

Embodiment 247. The pharmaceutical composition of embodiment 189, wherein the compound is:

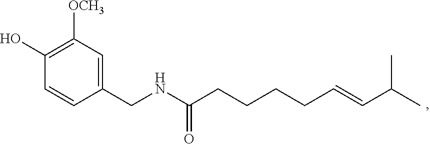 [SMILES: COc1cc(CNC(=O)CCCC/C=C/C(C)C)ccc1O]

or an isotopic variant thereof; or a metabolite, pharmaceutically acceptable salt, solvate, or hydrate thereof.

Embodiment 248. A method of treating or preventing a capsaicin-responsive disease or disorder, comprising administering to a subject in need thereof the pharmaceutical composition of any one of embodiments 189-247.

Embodiment 249. The method of embodiment 248, wherein the disease or disorder is a cell proliferative disease, obesity, diabetes, a bacterial infection, a cardiovascular disease, a neurodegenerative disease or disorder, an inflammatory disease or disorder, a comorbidity, an autoimmune disease, hypercholesterolemia or a mood disorder.

Embodiment 250. The method of embodiment 249, wherein the mood disorder is depression.

Embodiment 251. The method of embodiment 249, wherein the inflammatory disease disorder is rheumatoid arthritis.

Embodiment 252. The method of embodiment 249, wherein the cell proliferative disease is a cancer.

Embodiment 253. The method of embodiment 249, wherein the disorder is diabetes or obesity.

Embodiment 254. The method of embodiment 249, wherein the comorbidity is chronic kidney disease, stroke, congestive heart failure, dementia, schizophrenia, hepatitis, autism spectrum disorder, HIV, or a combination thereof.

Embodiment 255. A composition, comprising:
(i) a compound of structural Formula (I):

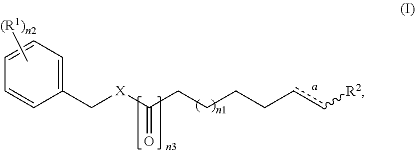

or an isotopic variant thereof; or a metabolite, pharmaceutically acceptable salt, solvate, or hydrate thereof,
   wherein:
[symbol] is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
(ii) a transient receptor potential cation channel subfamily V member 1 (TrpV1) partial agonist; and
( iii) at least one excipient.

Embodiment 256. The composition of embodiment 255, wherein the compound of structural Formula (I), or an isotopic variant thereof; or a metabolite, pharmaceutically acceptable salt, solvate, or hydrate thereof, is capsaicin.

Embodiment 257. The composition of any one of embodiments 255-256, wherein the TrpV1 partial agonist is a fatty acid.

Embodiment 258. The composition of any one of embodiments 255-257, wherein the TrpV1 partial agonist is a polyunsaturated fatty acid (PUFA).

Embodiment 259. The composition of any one of embodiments 255-258, wherein the PUFA is a n-3 PUFA.

Embodiment 260. The composition of any one of embodiments 255-259, wherein the PUFA is a n-6 PUFA.

Embodiment 261. The composition of any one of embodiments 255-260, wherein the TrpV1 partial agonist comprises DHA (docosahexaenoic acid), EPA (eicosapentaenoic acid), LNA (linolenic acid), LA (linoleic acid), or combinations thereof.

Embodiment 262. The composition of any one of embodiments 255-261, wherein the TrpV1 partial agonist comprises a free fatty acid, ester, triglyceride, or combination thereof of DHA (docosahexaenoic acid), EPA (eicosapentaenoic acid), LNA (linolenic acid), or LA (linoleic acid).

Embodiment 263. A composition, comprising:
(i) a compound of structural Formula (I):

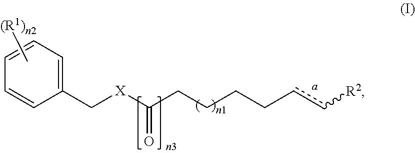

or an isotopic variant thereof; or a metabolite, pharmaceutically acceptable salt, solvate, or hydrate thereof,
   wherein:
[symbol] is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
(ii) a transient receptor potential cation channel subfamily V member 1 (TrpV1) antagonist; and
(iii) at least one excipient.

Embodiment 264. The composition of embodiment 263, wherein the compound of structural Formula (I), or an isotopic variant thereof; or a metabolite, pharmaceutically acceptable salt, solvate, or hydrate thereof, is capsaicin.

Embodiment 265. The composition of any one of embodiments 263-264, wherein the TrpV1 antagonist is a fatty acid.

Embodiment 266. The composition of any one of embodiments 263-265, wherein the TrpV1 antagonist is a polyunsaturated fatty acid (PUFA).

Embodiment 267. The composition of any one of embodiments 263-266, wherein the PUFA is a n-3 PUFA.

Embodiment 268. The composition of any one of embodiments 263-267, wherein the PUFA is a n-6 PUFA.

Embodiment 269. The composition of any one of embodiments 263-268, wherein the TrpV1 antagonist comprises DHA (docosahexaenoic acid), EPA (eicosapentaenoic acid), LNA (linolenic acid), LA (linoleic acid), petroselinic acid, oleic acid, or combinations thereof

Embodiment 270. The composition of any one of embodiments 263-269, wherein the TrpV1 antagonist comprises a free fatty acid, ester, triglyceride, or combination thereof of DHA (docosahexaenoic acid), EPA (eicosapentaenoic acid), LNA (linolenic acid), LA (linoleic acid), petroselinic acid, or oleic acid.

Embodiment 271. The composition of any one of embodiments 263-270, wherein the TrpV1 antagonist is a synthetic TrpV1 antagonist.

Embodiment 272. The composition of any one of embodiments 263-271, wherein the TrpV1 antagonist does not cause hyperthermia.

Embodiment 273. The composition of any one of embodiments 263-272, wherein the TrpV1 antagonist is

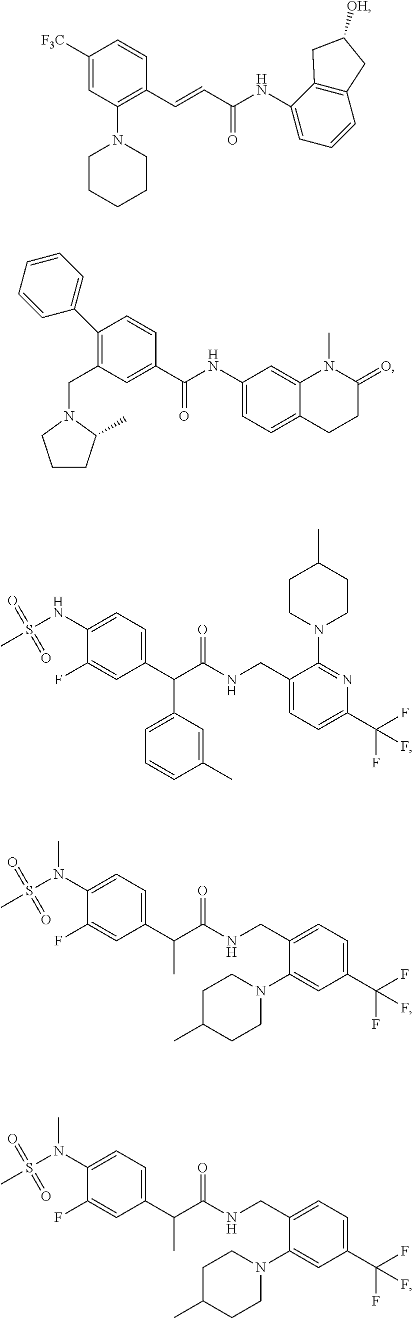 [SMILES: O=C(/C=C/c1ccc(C(F)(F)F)cc1N1CCCCC1)Nc1cccc2c1C[C@H](O)C2 ; C[C@@H]1CCCN1Cc1cc(C(=O)Nc2ccc3c(c2)N(C)C(=O)CC3)ccc1-c1ccccc1 ; Cc1cccc(C(C(=O)NCc2ccc(C(F)(F)F)nc2N2CCC(C)CC2)c2ccc(NS(C)(=O)=O)c(F)c2)c1 ; CC1CCN(c2cc(C(F)(F)F)ccc2CNC(=O)C(C)c2ccc(N(C)S(C)(=O)=O)c(F)c2)CC1 ; CC1CCN(c2cc(C(F)(F)F)ccc2CNC(=O)C(C)c2ccc(N(C)S(C)(=O)=O)c(F)c2)CC1]

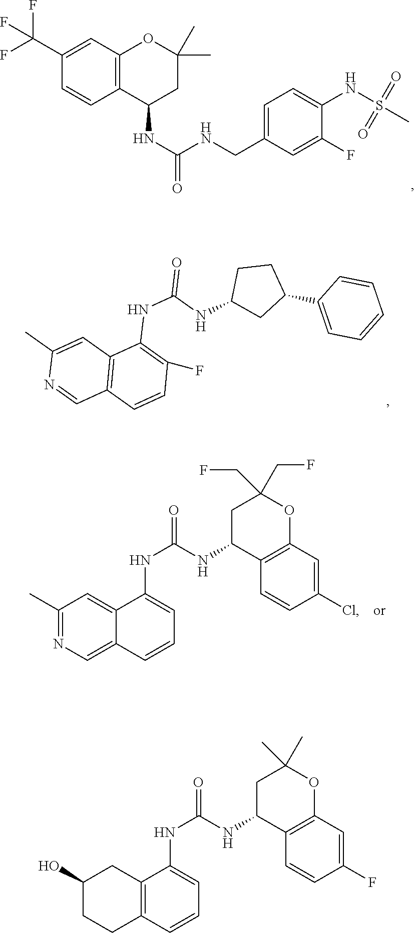

or an isotopic variant thereof; or a metabolite, pharmaceutically acceptable salt, solvate, or hydrate thereof.

Embodiment 274. A composition, comprising:
(i) a compound of structural Formula (I):

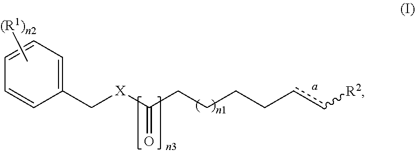

or an isotopic variant thereof; or a metabolite, pharmaceutically acceptable salt, solvate, or hydrate thereof,
   wherein:
[symbol] is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;

(ii) a transient receptor potential cation channel subfamily V member 1 (TrpV1) inverse agonist; and
(iii) at least one excipient.

Embodiment 275. The composition of embodiment 274, wherein the compound of structural Formula (I), or an isotopic variant thereof; or a metabolite, pharmaceutically acceptable salt, solvate, or hydrate thereof, is capsaicin.

Embodiment 276. The composition of any one of embodiments 274-275, wherein the TrpV1 inverse agonist is a fatty acid.

Embodiment 277. The composition of any one of embodiments 274-276, wherein the TrpV1 inverse agonist is a polyunsaturated fatty acid (PUFA).

Embodiment 278. The composition of any one of embodiments 274-277, wherein the TrpV1 inverse agonist is anandamide or derivative thereof.

### I. EXAMPLES

Example 1: The effect of capsaicin and a TrpV1 inhibitor on Resting Metabolic Weight.

Detailed Description: This study is a double-blind, placebo-controlled, single center, randomized, parallel arm clinical trial to test the impact of capsaicin and a TrpV1 inhibitor in a 1:1 ratio (placebo, 40% (w/v)) ingested for 4 weeks on resting metabolic rate and fat oxidation measured by indirect calorimetry.

Primary Outcome

| | |
|---|---|
| Measures | The consumption of capsaicin and a TrpV1 inhibitor will significantly increase resting |
| | energy expenditure and fat oxidation [ Time Frame: 4 weeks ]. |

Descriptive Information

| | |
|---|---|
| Brief Title | A 4 week intake of drug combination to find a natural substance that may modify energy |
| | balance and may enhance health in combination with lifestyle changes with possible |
| | decrease in body weight. |
| Official Title | Effect of 4-week Capsaicin and a TrpV1 Inhibitor Ingestion on Resting Metabolic Rate: |
| | A Double-blind Randomized Parallel Arm Study |
| Brief Summary | A 4 week intake of drug to find a natural substance that may modify energy balance and |
| | may enhance health in combination with lifestyle changes with possible decrease in body |
| | weight |
| Study Type | Interventional |
| Study Phase | Phase 2 |
| Study Design | Allocation: Randomized |
| | Intervention Model: Parallel Assignment |
| | Masking: Double (Participant, Investigator) |
| | Primary Purpose: Prevention |
| Condition | Obesity, Weight |
| Intervention | Other: Capsaicin in combination a TrpV1 inhibitor |
| | Capsules will contain 40% (w/v) capsaicin and a TrpV1 inhibitor in a 1:1 ratio |
| Study Arms | Placebo Comparator: Sugar pill (Placebo 0 mg/d) |
| | 0 mg/d sugar pill |
| | Intervention: Other: Capsaicin and TrpV1 combination |
| | Active Comparator: Capsaicin and TrpV1 combination |
| | Drug 40% (w/v) including Placebo |
| | Intervention: Other: Capsaicin and TrpV1 combination |
| | Active Comparator: 40% (w/v) capsaicin and a TrpV1 inhibitor in a 1:1 ratio |
| | Drug including Placebo |
| | Intervention: Other: capsaicin and a TrpV1 inhibitor combination |

Recruitment Information

| | |
|---|---|
| Eligibility | Inclusion Criteria: |
| Criteria | Men between 20-60 years old |
| | Healthy as assessed by medical history and standard medical exam |
| | Weight-stable |
| | Body mass index of 25 to 34.9 kg/m² |
| | Non-smoker |
| | Sedentary lifestyle: not being physically active greater than 3 days/week for 20 |
| | min each time for the previous 6 months, and not participating in regular resistance |
| | exercise. |
| | Exclusion Criteria: |
| | Subjects enrolled in a diet to increase or decrease body weight |
| | Special diet or food aversions to common foods |
| | Has allergy to chilli pepper |
| | Eating chilli peppers on a daily basis |
| | Usually consuming more than 2 cups of tea or coffee/day |
| | Usually consuming more than 4 cans of caffeinated soft drinks a day |
| | Usually consuming more than 3 standard alcohol drinks/day |
| | Regular use of medications (weight loss drugs, drugs affecting energy metabolism, |
| | drugs for depression) |
| | Usual intake of illicit substances |
| | Claustrophobia |
| | Participating or having participated in another clinical trial during the last 4 weeks |
| | prior to the beginning of this study |
| Sex/Gender | Sexes Eligible for Study: All |
| Ages | 20 years to 60 years (Adult, Older Adult) |
| Accepts Healthy | Yes |
| Volunteers | |

Although the disclosure has been described with reference to the above example, it will be understood that modifications and variations are encompassed within the spirit and scope of the disclosure. Accordingly, the disclosure is limited only by the following claims.

What is claimed is:

1. A composition, comprising:
(i) a compound of structural Formula (I): 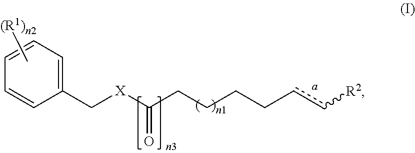
or an isotopic variant thereof; or a metabolite, pharmaceutically acceptable salt, solvate, or hydrate thereof,
wherein: [symbol]
is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
(ii) at least one additional therapeutic agent selected from:
a fatty acid amide hydrolase (FAAH) receptor inhibitor, and
a specialized pro-resolving mediator (SPM), or a combination thereof; and
(iii) at least one excipient.

2. A composition, comprising:
(i) a compound of structural Formula al: 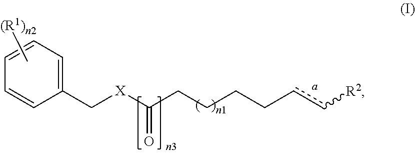
or an isotopic variant thereof; or a metabolite, pharmaceutically acceptable salt, solvate, or hydrate thereof,
wherein: [symbol]
is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
(ii) a transient receptor potential cation channel subfamily V member 1 (TrpV1) modulator; and
(iii) at least one excipient,
wherein when the compound of structural Formula (I), or an isotopic variant thereof; or a metabolite, pharmaceutically acceptable salt, solvate, or hydrate thereof, is capsaicin, then the capsaicin is present in the composition in an amount of greater than about 40% (w/v).

3. The composition of claim 2, wherein the capsaicin is present in the composition in an amount of from about 40% (w/v) to about 50% (w/v).

4. A composition, comprising:
(i) a compound of structural Formula (I): 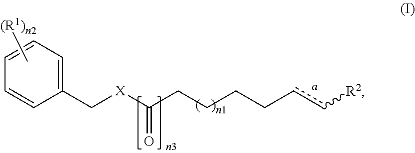
or an isotopic variant thereof; or a metabolite, pharmaceutically acceptable salt, solvate, or hydrate thereof,
wherein: [symbol]
is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
(ii) a TrpV1 modulator; and
(iii) at least one excipient,
wherein the compound of Formula (I), isotopic variant thereof; or a metabolite, pharmaceutically acceptable salt, solvate, or hydrate thereof, is not capsaicin; or
wherein the TrpV1 modulator is not a fatty acid.

5. The composition of any one of claims 1-4, wherein the at least one TrpV1 modulator is a TrpV1 antagonist, a TrpV1 competitive agonist, or combination thereof.

6. The composition of claim 4, wherein the at least one TrpV1 modulator is a triglyceride or spilanthol.

7. The composition of any one of claim 1-3 or 5, wherein the at least one TrpV1 modulator is a fatty acid, triglyceride, or spilanthol.

8. The composition of claim 7, wherein the fatty acid is a polyunsaturated fatty acid (PUFA).

9. The composition of claim 8, wherein the PUFA is anandamide.

10. The composition of claim 7, wherein the fatty acid is an omega-9 fatty acid.

11. The composition of claim 10, wherein the fatty acid is oleic acid or erucic acid.

12. The composition of claim 1, wherein the at least one additional therapeutic agent is a FAAH receptor inhibitor.

13. The composition of claim 1, wherein the at least one additional therapeutic agent is an SPM.

14. The composition of claim 13, wherein the SPM is maresin.

15. The composition of any one of claims 1-14, wherein the composition is in a form for oral dosing or administration.

16. The composition of any one of claims 4-15, wherein the compound of Formula (I), or an isotopic variant thereof; or a metabolite, pharmaceutically acceptable salt, solvate, or hydrate thereof is present in the composition in an amount of greater than about 10% (w/v).

17. The composition of any one of claims 4-15, wherein the compound of Formula (I), or an isotopic variant thereof; or a metabolite, pharmaceutically acceptable salt, solvate, or hydrate thereof is present in the composition in an amount of from about 40% (w/v) to about 50% (w/v).

18. The composition of any one of claims 1-17, wherein the compound of structural Formula (I) has structural Formula (I-A): 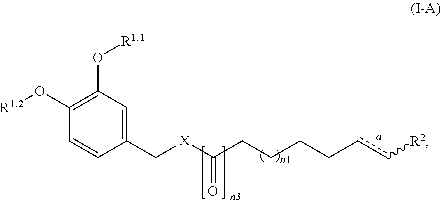
or an isotopic variant thereof; or a metabolite, pharmaceutically acceptable salt, solvate, or hydrate thereof,
wherein:
R^{1.1} is hydrogen, halogen, —OR^{1.1A}, substituted or unsubstituted alkyl, or substituted or unsubstituted heteroalkyl;
R^{1.2} is hydrogen, halogen, —OR^{1.2A}, substituted or unsubstituted alkyl, or substituted or unsubstituted heteroalkyl; and
R^{1.1A} and R^{1.2A} are independently hydrogen, —CF₃, —CCl₃, —CBr₃, —CI₃, —COOH, —CONH₂, substituted or unsubstituted alkyl, or substituted or unsubstituted heteroalkyl.

19. The pharmaceutical composition of claim 18, wherein:
R^{1.1} is CH_{3;} and
R^{1.2} is OH.

20. The composition of claim 18, wherein X is O.

21. The composition of claim 18, wherein X is NH.

22. The composition of any one of claims 18-21, wherein [symbol] is a double bond.

23. The composition of any one of claims 18-21, wherein [symbol] is a single bond.

24. The composition of any one of claims 18-23, wherein n1 is 2.

25. The composition of any one of claims 18-23, wherein n1 is 1.

26. The composition of any one of claims 18-23, wherein n1 is 0.

27. The composition of any one of claims 18-26, wherein R² is unsubstituted alkyl.

28. The composition of any one of claims 18-26, wherein R² is —CH(CH₃)₂ or —CH₃.

29. The composition of any one of claims 1-18, wherein the compound is at least one of: 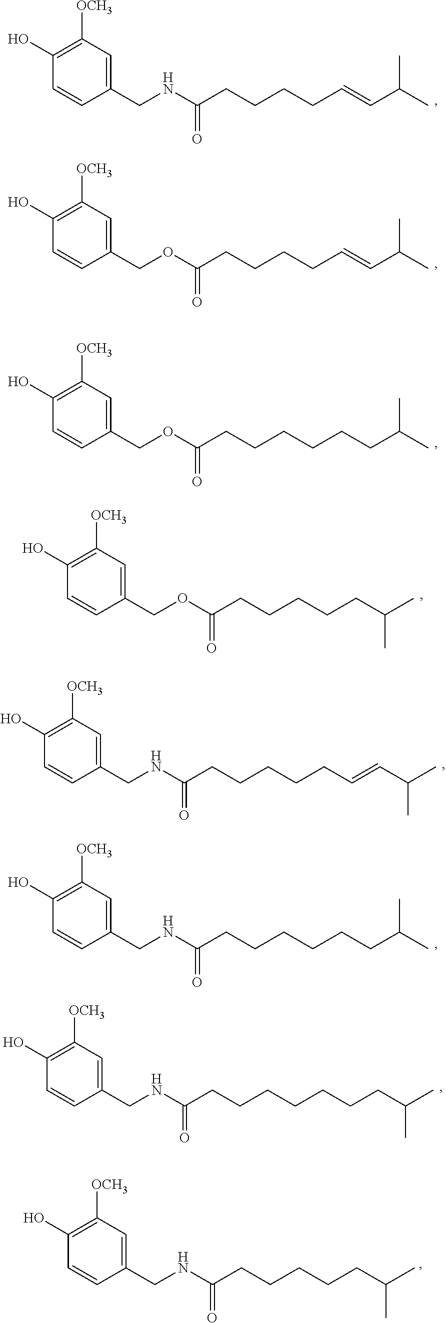 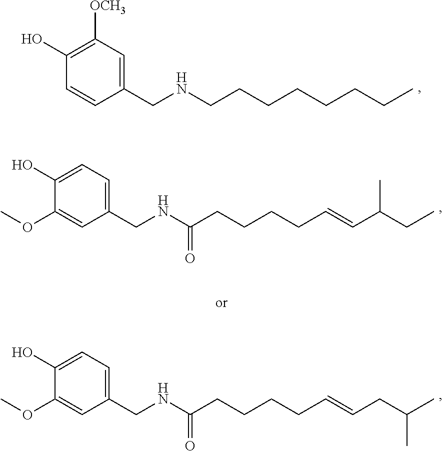
or an isotopic variant thereof; or a metabolite, pharmaceutically acceptable salt, solvate, or hydrate thereof.

30. The composition of any one of claims 1-18, wherein the compound is: 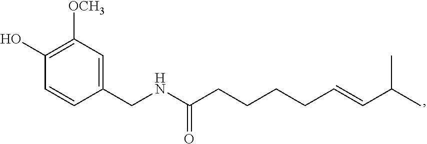 [SMILES: COc1cc(CNC(=O)CCCC/C=C/C(C)C)ccc1O]
or an isotopic variant thereof; or a metabolite, pharmaceutically acceptable salt, solvate, or hydrate thereof.

31. A composition, comprising:
(i) a compound of structural Formula (I): 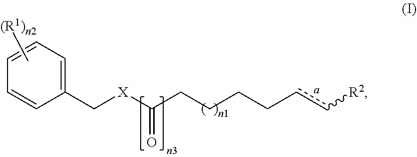
or an isotopic variant thereof; or a metabolite, pharmaceutically acceptable salt, solvate, or hydrate thereof,
wherein: [symbol]
is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
(ii) a transient receptor potential cation channel subfamily V member 1 (TrpV1) partial agonist; and
(iii) at least one excipient.

32. The composition of claim 31, wherein the compound of structural Formula (I), or an isotopic variant thereof; or a metabolite, pharmaceutically acceptable salt, solvate, or hydrate thereof, is capsaicin.

33. The composition of claim 31, wherein the TrpV1 partial agonist is a fatty acid.

34. The composition of claim 31, wherein the TrpV1 partial agonist is a polyunsaturated fatty acid (PUFA).

35. The composition of claim 34, wherein the PUFA is a n-3 PUFA.

36. The composition of claim 34, wherein the PUFA is a n-6 PUFA.

37. The composition of claim 31, wherein the TrpV1 partial agonist comprises DHA (docosahexaenoic acid), EPA (eicosapentaenoic acid), LNA (linolenic acid), LA (linoleic acid), or combinations thereof.

38. The composition of claim 31, wherein the TrpV1 partial agonist comprises a free fatty acid, ester, triglyceride, or combination thereof of DHA (docosahexaenoic acid), EPA (eicosapentaenoic acid), LNA (linolenic acid), or LA (linoleic acid).

39. A composition, comprising:
(i) a compound of structural Formula (I): 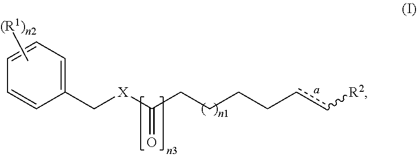
or an isotopic variant thereof; or a metabolite, pharmaceutically acceptable salt, solvate, or hydrate thereof,
wherein: [symbol]
is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
(ii) a transient receptor potential cation channel subfamily V member 1 (TrpV1) antagonist; and
(iii) at least one excipient.

40. The composition of claim 39, wherein the compound of structural Formula (I), or an isotopic variant thereof; or a metabolite, pharmaceutically acceptable salt, solvate, or hydrate thereof, is capsaicin.

41. The composition of claim 39, wherein the TrpV1 antagonist is a fatty acid.

42. The composition of claim 39, wherein the TrpV1 antagonist is a polyunsaturated fatty acid (PUFA).

43. The composition of claim 42, wherein the PUFA is a n-3 PUFA.

44. The composition of claim 42, wherein the PUFA is a n-6 PUFA.

45. The composition of claim 39, wherein the TrpV1 antagonist comprises DHA (docosahexaenoic acid), EPA (eicosapentaenoic acid), LNA (linolenic acid), LA (linoleic acid), petroselinic acid, oleic acid, or combinations thereof

46. The composition of claim 39, wherein the TrpV1 antagonist comprises a free fatty acid, ester, triglyceride, or combination thereof of DHA (docosahexaenoic acid), EPA (eicosapentaenoic acid), LNA (linolenic acid), LA (linoleic acid), petroselinic acid, or oleic acid.

47. The composition of claim 39, wherein the TrpV1 antagonist is a synthetic TrpV1 antagonist.

48. The composition of claim 39, wherein the TrpV1 antagonist does not cause hyperthermia.

49. The composition of claim 39, wherein the TrpV1 antagonist is 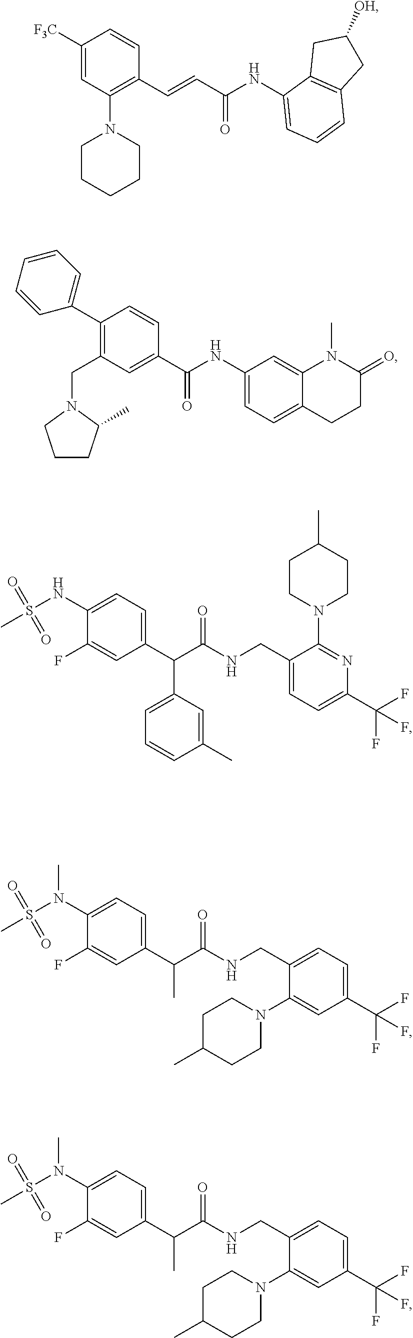 [SMILES: O=C(/C=C/c1ccc(C(F)(F)F)cc1N1CCCCC1)Nc1cccc2c1C[C@H](O)C2 ; C[C@@H]1CCCN1Cc1cc(C(=O)Nc2ccc3c(c2)N(C)C(=O)CC3)ccc1-c1ccccc1 ; Cc1cccc(C(C(=O)NCc2ccc(C(F)(F)F)nc2N2CCC(C)CC2)c2ccc(NS(C)(=O)=O)c(F)c2)c1 ; CC1CCN(c2cc(C(F)(F)F)ccc2CNC(=O)C(C)c2ccc(N(C)S(C)(=O)=O)c(F)c2)CC1 ; CC1CCN(c2cc(C(F)(F)F)ccc2CNC(=O)C(C)c2ccc(N(C)S(C)(=O)=O)c(F)c2)CC1] 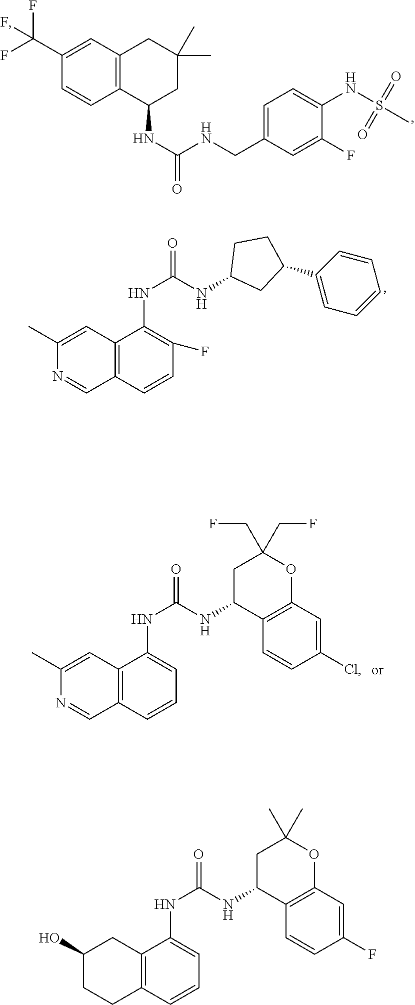
or an isotopic variant thereof; or a metabolite, pharmaceutically acceptable salt, solvate, or hydrate thereof.

50. A composition, comprising:
(i) a compound of structural Formula (I): 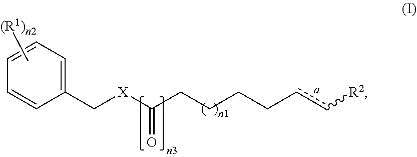
or an isotopic variant thereof; or a metabolite, pharmaceutically acceptable salt, solvate, or hydrate thereof,
wherein: [symbol]
is a single bond or double bond;
n1 and n2 are independently an integer from 0 to 2;
n3 is 0 or 1;
X is O, NH, or S;
R¹ is independently hydrogen, halogen, —OR^{1A}, substituted or unsubstituted alkyl, or substituted or unsubstituted heteroalkyl;
R² is hydrogen or substituted or unsubstituted C₁₋₆ alkyl; and
R^{1A} is hydrogen, —CF₃, —CCl₃, —CBr₃, —CI₃, —COOH, —CONH₂, substituted or unsubstituted alkyl, or substituted or unsubstituted heteroalkyl;
(ii) a transient receptor potential cation channel subfamily V member 1 (TrpV1) inverse agonist; and
(iii) at least one excipient.

51. The composition of claim 50, wherein the compound of structural Formula (I), or an isotopic variant thereof; or a metabolite, pharmaceutically acceptable salt, solvate, or hydrate thereof, is capsaicin.

52. The composition of claim 50, wherein the TrpV1 inverse agonist is a fatty acid.

53. The composition of claim 50, wherein the TrpV1 inverse agonist is a polyunsaturated fatty acid (PUFA).

54. The composition of claim 50, wherein the TrpV1 inverse agonist is anandamide or derivative thereof.

55. A method of treating or preventing a capsaicin-responsive disease or disorder, comprising administering to a subject in need thereof the composition of any one of claims 1-54.

56. The method of claim 55, wherein the disease or disorder is a cell proliferative disease, obesity, diabetes, a bacterial infection, a cardiovascular disease, a neurodegenerative disease or disorder, an inflammatory disease or disorder, a comorbidity, an autoimmune disease, hypercholesterolemia or a mood disorder.

57. The method of claim 56, wherein the mood disorder is depression.

58. The method of claim 56, wherein the inflammatory disease or disorder is rheumatoid arthritis.

59. The method of claim 56, wherein the cell proliferative disease is a cancer.

60. The method of claim 56, wherein the disorder is diabetes or obesity.

61. The method of claim 56, wherein the comorbidity is chronic kidney disease, stroke, congestive heart failure, dementia, schizophrenia, hepatitis, autism spectrum disorder, HIV, or a combination thereof.

62. The method of any one of claims 55-61, wherein the compound of structural Formula (I) or an isotopic variant thereof; or a metabolite, pharmaceutically acceptable salt, solvate, or hydrate thereof, is administered orally.

63. The method of any one of claims 55-61, wherein the compound of structural Formula (I) or an isotopic variant thereof; or a metabolite, pharmaceutically acceptable salt, solvate, or hydrate thereof, is administered orally via a suspension or solution.

64. The method of any one of claims 55-61, wherein the compound of structural Formula (I) or an isotopic variant thereof; or a metabolite, pharmaceutically acceptable salt, solvate, or hydrate thereof, is administered orally via a solid dosage form.

65. The method of claim 64, wherein the form is a tablet or capsule.

66. The method of any one of claims 55-61, wherein the compound of structural Formula (I) or an isotopic variant thereof; or a metabolite, pharmaceutically acceptable salt, solvate, or hydrate thereof, is administered by inhalation, nebulization, or nasal delivery.

67. The method of claim 66, wherein the compound of structural Formula (I) or an isotopic variant thereof; or a metabolite, pharmaceutically acceptable salt, solvate, or hydrate thereof, is administered by inhalation via a vaporizer.

68. The method of any one of claims 55-61, wherein the compound of structural Formula (I) or an isotopic variant thereof; or a metabolite, pharmaceutically acceptable salt, solvate, or hydrate thereof, is administered parenterally.

69. The method of any one of claims 55-61, wherein the compound of structural Formula (I) or an isotopic variant thereof; or a metabolite, pharmaceutically acceptable salt, solvate, or hydrate thereof, is administered via parenteral injection, infusion, or implantation.

70. The method of claim 69, wherein the parenteral injection is intravenous, intramuscular, subcutaneous, or intradermal.

71. The method of any one of claims 55-61, wherein the compound of structural Formula (I) or an isotopic variant thereof; or a metabolite, pharmaceutically acceptable salt, solvate, or hydrate thereof, is administered via a suppository.

72. The method of any one of claims 55-61, wherein the compound of structural Formula (I) or an isotopic variant thereof; or a metabolite, pharmaceutically acceptable salt, solvate, or hydrate thereof, is administered cutaneous or transdermal delivery.

73. The method of any one of claims 55-61, wherein the compound of structural Formula (I) or an isotopic variant thereof; or a metabolite, pharmaceutically acceptable salt, solvate, or hydrate thereof, is administered by sublingual or buccal delivery.

74. The method of any one of claims 55-61, wherein the compound of Formula (I), or a pharmaceutically acceptable salt thereof, and the at least one additional therapeutic agent are administered simultaneously, approximately simultaneously, or sequentially, in any order.

75. The method of claim 74, wherein the compound of Formula (I), or a pharmaceutically acceptable salt thereof, and the at least one additional therapeutic agent are administered simultaneously or approximately simultaneously.

76. The method of claim 74, wherein the compound of Formula (I), or a pharmaceutically acceptable salt thereof, and the at least one additional therapeutic agent are administered sequentially.

77. The method of claim 75, wherein the compound of Formula (I), or a pharmaceutically acceptable salt thereof, is released before the at least one additional therapeutic agent.

78. The method of claim 75, wherein the compound of Formula (I), or a pharmaceutically acceptable salt thereof, is released after the at least one additional therapeutic agent.
